(19) United States
(12) Patent Application Publication
(10) Pub. No.: US 2024/0350813 A1
(43) Pub. Date: Oct. 24, 2024

(54) SYSTEMS AND METHODS FOR AUTOMATICALLY MODIFYING ONE OR MORE GRAPHICAL USER INTERFACE (GUI) COMPONENTS OF AN IMPLANTABLE MEDICAL DEVICE (IMD)-RELATED APPLICATION BY MONITORING AND ANALYZING PATIENT INTERACTION

(71) Applicant: Advanced Neuromodulation Systems, Inc., Plano, TX (US)

(72) Inventors: Scott DeBates, Frisco, TX (US); Binesh Balasingh, Prosper, TX (US); Mary Khun Hor-Lao, Prosper, TX (US); Douglas Alfred Lautner, Frisco, TX (US); Amanda Jean Richardson, Frisco, TX (US); Navin Dabhi, Frisco, TX (US)

(21) Appl. No.: 18/642,541

(22) Filed: Apr. 22, 2024

Related U.S. Application Data
(60) Provisional application No. 63/497,446, filed on Apr. 21, 2023.

(51) Int. Cl. A61N 1/372; A61N 1/02; A61N 1/36; G06F 3/04845; G06F 3/0488

(52) U.S. Cl. CPC A61N 1/37247; A61N 1/025; A61N 1/36067; A61N 1/36135; G06F 3/04845; G06F 3/0488

(57) ABSTRACT

The present application is generally related to automatic modification of one or more graphical user interface (GUI) components of an implantable medical device (IMD)-related application by monitoring and analyzing patient interaction with the IMD-related application.

### PRIORITY AND CROSS-REFERENCE TO RELATED APPLICATION(S)

Not applicable.

### TECHNICAL FIELD

The present application is generally related to automatic modification of one or more graphical user interface (GUI) components of an implantable medical device (IMD)-related application by monitoring and analyzing patient interaction with the IMD-related application.

### BACKGROUND

Implantable medical devices have changed how medical care is provided to patients having a variety of chronic illnesses and disorders. For example, implantable cardiac devices improve cardiac function in patients with heart disease by improving quality of life and reducing mortality rates. Respective types of implantable neurostimulators provide a reduction in pain for chronic pain patients and reduce motor difficulties in patients with Parkinson's disease and other movement disorders. A variety of other medical devices are proposed and are in development to treat other disorders in a wide range of patients.

Many implantable medical devices and other personal medical devices are programmed by a physician or other clinician to optimize the therapy provided by a respective device to an individual patient. Typically, the programming occurs using short-range communication links (e.g., inductive wireless telemetry) in an in-person or in-clinic setting. Since such communications typically require close immediate contact, there is only an extremely small likelihood of a third-party establishing a communication session with the patient's implanted device without the patient's knowledge.

Remote patient care is a healthcare delivery method that aims to use technology to provide patient health outside of a traditional clinical setting (e.g., in a doctor's office or a patient's home). It is widely expected that remote patient care may increase access to care and decrease healthcare delivery costs.

### BRIEF DESCRIPTION OF THE DRAWINGS

Embodiments of the present disclosure are illustrated by way of example, and not by way of limitation, in the Figures of the accompanying drawings in which like references indicate similar elements. It should be noted that different references to “an” or “one” embodiment in this disclosure are not necessarily to the same embodiment, and such references may mean at least one. Further, when a particular feature, structure, or characteristic is described in connection with an embodiment, it is submitted that it is within the knowledge of one skilled in the art to effectuate such feature, structure, or characteristic in connection with other embodiments whether or not explicitly described.

The accompanying drawings are incorporated into and form a part of the specification to illustrate one or more exemplary embodiments of the present disclosure. Various advantages and features of the disclosure will be understood from the following Detailed Description taken in connection with the appended claims and with reference to the attached drawing Figures in which:

FIG. 1A depicts an example architecture of a system configured to support remote patient therapy as part of an integrated remote care service session in a virtual clinic environment that may be deployed in a cloud-centric digital health implementation wherein one or more embodiments of the present patent disclosure may be practiced in accordance with the teachings herein;

FIG. 1B depicts an example network environment wherein the remote care service architecture of FIG. 1A may be implemented according to a representative embodiment;

FIG. 2 depicts a flowchart illustrative of blocks, steps and/or acts that may be (re) combined in one or more arrangements with or without additional flowcharts of the present disclosure for facilitating remote care therapy in a secure network environment for purposes of some embodiments;

FIG. 3 depicts a flowchart illustrative of blocks, steps and/or acts that may be implemented for establishing a communication session with an implantable medical device;

FIGS. 4A and 4B depicts flowcharts illustrative of a remote care scenario involving an example digital health network architecture wherein an integrated remote care session may be established between a patient and a clinician operating respective controller devices for purposes of some embodiments of the present disclosure;

FIGS. 5A and 5B depict representations of an example user interface and associated dialog boxes provided with a clinician programmer device for selecting different therapy applications and/or service modes for purposes of some embodiments of the present disclosure;

FIG. 6 depicts a representation of an example user interface provided with a clinician programmer device for facilitating controls with respect to an AV communication session and a remote therapy session in an integrated remote care service application for purposes of some embodiments of the present disclosure;

FIG. 7 depicts a block diagram of an external device that may be configured as a clinician programmer device, a patient controller device or an authorized third-party device operative in a digital health network architecture for purposes of some example embodiments of the present disclosure;

FIG. 8 depicts a block diagram illustrating additional details of a patient controller device operative in a digital health network architecture for purposes of some example embodiments of the present disclosure;

FIG. 9 depicts a block diagram illustrating additional details of a clinician programmer device operative in a digital health network architecture for purposes of some example embodiments of the present disclosure;

FIG. 10 depicts a block diagram of an IMD and associated system that may be configured for facilitating a remote care therapy application and/or a local therapy session for purposes of some example embodiments of the present disclosure;

FIGS. 11A-11C depict representations of an example user interface and associated dialog boxes provided with a patient controller device for selecting different therapy applications and/or service modes and for facilitating controls with respect to an AV communication session and a remote therapy session in an integrated remote care service application for purposes of some embodiments of the present disclosure;

FIG. 12 depicts an example cloud-centric digital healthcare network architecture including one or more virtual clinic platforms, patient report processing platforms and remote data logging platforms wherein some example embodiments of the present disclosure may be implemented;

FIG. 13 depicts a block diagram of a system, apparatus or a computer-implemented platform that may be configured as a virtual clinic for purposes of some example embodiments of the present disclosure;

FIG. 14 depicts a flowchart illustrative of blocks, steps and/or acts that may be (re) combined in one or more arrangements with or without additional flowcharts of the present disclosure for facilitating enhanced functionalities in a digital healthcare network according to some example embodiments;

FIG. 15 depicts a flowchart illustrative of blocks, steps and/or acts that may be (re) combined in one or more arrangements with or without additional flowcharts of the present disclosure for facilitating secure switching/redirection of an audio/video (A/V) session between terminal endpoints during an integrated remote therapy session including programming in a digital healthcare network according to some example embodiments;

FIGS. 16 and 17 depict flowcharts illustrative of blocks, steps and/or acts that may be (re) combined in one or more arrangements with or without additional flowcharts of the present disclosure for facilitating remote analysis and assistance of an implantable device and/or associated patient controller deployed in a digital healthcare network according to some example embodiments;

FIGS. 18A-18C depict flowcharts illustrative of blocks, steps and/or acts that may be (re) combined in one or more arrangements with or without additional flowcharts of the present disclosure for facilitating contextual notification and enablement of third-party device communication in an ongoing remote therapy session including a remote programming session and associated A/V communication session in a digital healthcare network according to some example embodiments;

FIG. 19 depicts a flowchart illustrative of blocks, steps and/or acts that may be (re) combined in one or more arrangements with or without additional flowcharts of the present disclosure for facilitating privacy policy control for multiple parties participating in a remote programming/therapy session effectuated by a virtual clinic deployed in a digital healthcare network according to some example embodiments;

FIG. 20 depicts an example network architecture including a virtual clinic for facilitating A/V session redirection/switching during an integrated remote therapy session according to an implementation of the present disclosure;

FIGS. 21A and 21B depict a flowchart illustrative of blocks, steps and/or acts of a process with additional details for effectuating redirection/switching of an A/V session to an auxiliary/secondary device associated with a patient and/or a clinician according to an implementation of the present disclosure;

FIGS. 22A and 22B depict a flowchart illustrative of blocks, steps and/or acts of a process with additional details for effectuating a remote assistance procedure with respect to a patient controller device according to an implementation of the present disclosure;

FIGS. 23A-1 and 23A-2 together depict various screenshot views relating to effectuating a remote assistance procedure according to some implementations of the present disclosure;

FIG. 23B depicts various screenshot views relating to effectuating a remote assistance procedure according to some implementations of the present disclosure;

FIGS. 23C-1 and 23C-2 together depict various screenshot views relating to effectuating a remote assistance procedure according to some implementations of the present disclosure;

FIG. 23D depicts various screenshot views relating to effectuating a remote assistance procedure according to some implementations of the present disclosure;

FIGS. 23E-1 and 23E-2 together depict various screenshot views relating to effectuating a remote assistance procedure according to some implementations of the present disclosure;

FIG. 24 depicts a flowchart illustrative of blocks, steps and/or acts of a process with additional details for effectuating a management process with respect to an authorized third-party device enabled to join an ongoing integrated remote therapy session according to an implementation of the present disclosure;

FIGS. 25A and 25B together depict an example network architecture including a virtual clinic for facilitating third-party enablement with respect to an ongoing integrated remote therapy session according to an implementation of the present disclosure;

FIG. 26 depicts screenshot views associated with a user interface of a third-party device having a remote monitoring application for joining a remote therapy session according to an implementation of the present disclosure;

FIGS. 27A and 27B together depict an example network architecture including a virtual clinic for facilitating privacy control with respect to third parties joining an ongoing integrated remote therapy session according to an implementation of the present disclosure;

FIG. 28 depicts an example neural network implementation for anonymizing patient/clinician images and data with respect to an A/V session joined by a third-party according to an embodiment of the present disclosure;

FIG. 29 depicts an example scheme for facilitating recording/storing of a remote therapy session having privacy policy controls for an A/V session according to an embodiment of the present disclosure;

FIGS. 30A and 30B together depict a sequence of UI screenshots of a patient's device effectuated for inviting a third-party user according to an example embodiment of the present disclosure;

FIGS. 31A and 31B together depict a sequence of UI screenshots of a third party's device invited by a patient for effectuating login and authorization according to an example embodiment of the present disclosure;

FIG. 32 depicts a message flow associated with initiating a multi-party session according to an example embodiment of the present disclosure;

FIG. 33 depicts a message flow associated with adding a health provider to a multi-party session according to an example embodiment of the present disclosure;

FIG. 34 depicts a message flow associated with adding a third-party user to a multi-party session according to an example embodiment of the present disclosure;

FIG. 35 depicts a flowchart illustrative of blocks, functions, steps and/or acts of a process for providing role-based UI control of a video session according to an example embodiment of the present disclosure;

FIG. 36 depicts an example role and hierarchical relationship scheme for assigning different temporal relations with respect to multiple parties in a multi-party session according to an example embodiment of the present disclosure; and

FIGS. 37-41 depict system components and flowcharts for adapting user interface components for an application for neurostimulation patients according to some representative embodiments.

### DETAILED DESCRIPTION

In the description herein for embodiments of the present disclosure, numerous specific details are provided, such as examples of circuits, devices, components and/or methods, to provide a thorough understanding of embodiments of the present disclosure. One skilled in the relevant art will recognize, however, that an embodiment of the disclosure can be practiced without one or more of the specific details, or with other apparatuses, systems, assemblies, methods, components, materials, parts, and/or the like set forth in reference to other embodiments herein. In other instances, well-known structures, materials, or operations are not specifically shown or described in detail to avoid obscuring aspects of embodiments of the present disclosure. Accordingly, it will be appreciated by one skilled in the art that the embodiments of the present disclosure may be practiced without such specific components. It should be further recognized that those of ordinary skill in the art, with the aid of the Detailed Description set forth herein and taking reference to the accompanying drawings, will be able to make and use one or more embodiments without undue experimentation.

Additionally, terms such as “coupled” and “connected,” along with their derivatives, may be used in the following description, claims, or both. It should be understood that these terms are not necessarily intended as synonyms for each other. “Coupled” may be used to indicate that two or more elements, which may or may not be in direct physical or electrical contact with each other, co-operate or interact with each other. “Connected” may be used to indicate the establishment of communication, i.e., a communicative relationship, between two or more elements that are coupled with each other. Further, in one or more example embodiments set forth herein, generally speaking, an electrical element, component or module may be configured to perform a function if the element may be programmed for performing or otherwise structurally arranged to perform that function.

Example embodiments described herein relate to aspects of implementations of an integrated digital health network architecture that may be effectuated as a convergence of various technologies involving diverse end user devices and computing platforms, heterogeneous network connectivity environments, agile software as a medical device (SaMD) deployments, data analytics, artificial intelligence and machine learning, secure cloud-centric infrastructures for supporting remote healthcare, etc. Some embodiments may be configured to support various types of healthcare solutions including but not limited to remote patient monitoring, integrated session management for providing telehealth applications as well as remote care therapy applications, personalized therapy based on advanced analytics of patient and clinician data, remote trialing of neuromodulation therapies, e.g., pain management/amelioration solutions, and the like. Whereas some example embodiments may be particularly set forth with respect to implantable pulse generator (IPG) or neuromodulator systems for providing therapy to a desired area of a body or tissue based on a suitable stimulation therapy application, such as spinal cord stimulation (SCS) systems or other neuromodulation systems, it should be understood that example embodiments disclosed herein are not limited thereto but have broad applicability. Some example remote care therapy applications may therefore involve different types of implantable devices such as neuromuscular stimulation systems and sensors, dorsal root ganglion (DRG) stimulation systems, deep brain stimulation systems, cochlear implants, retinal implants, implantable cardiac rhythm management devices, implantable cardioverter defibrillators, pacemakers, and the like, as well as implantable drug delivery/infusion systems, implantable devices configured to effectuate real-time measurement/monitoring of one or more physiological functions of a patient's body (i.e., patient physiometry), including various implantable biomedical sensors and sensing systems. Further, whereas some example embodiments of remote care therapy applications may involve implantable devices, additional and/or alternative embodiments may involve external personal devices and/or noninvasive/minimally invasive (NIMI) devices, e.g., wearable biomedical devices, transcutaneous/subcutaneous devices, etc., that may be configured to provide therapy to the patients analogous to the implantable devices. Accordingly, all such devices may be broadly referred to as “personal medical devices,” “personal biomedical instrumentation,” or terms of similar import, at least for purposes of some example embodiments of the present disclosure.

As used herein, a network element, platform or node may be comprised of one or more pieces of network equipment, including hardware and software that communicatively interconnects other equipment on a network (e.g., other network elements, end stations, etc.), and is adapted to host one or more applications or services, more specifically healthcare applications and services, with respect to a plurality of end users, e.g., patients, clinicians, respective authorized agents, third-party users such as caregivers, family relatives, etc. and associated client devices as well as other endpoints such as medical- and/or health-oriented Internet of Medical Things (IoMT) devices/sensors and/or other Industrial IoT-based entities. As such, some network elements may be operatively disposed in a cellular wireless or satellite telecommunications network, or a broadband wireline network, whereas other network elements may be disposed in a public packet-switched network infrastructure (e.g., the Internet or worldwide web, also sometimes referred to as the “cloud”), private packet-switched network infrastructures such as Intranets and enterprise networks, as well as service provider network infrastructures, any of which may span or involve a variety of access networks, backhaul and core networks in a hierarchical arrangement. In still further arrangements, one or more network elements may be disposed in cloud-based platforms or datacenters having suitable equipment running virtualized functions or applications, which may be configured for purposes of facilitating patient monitoring, remote therapy, other telehealth/telemedicine applications, etc. for purposes of one or more example embodiments set forth hereinbelow.

One or more embodiments of the present patent disclosure may be implemented using different combinations of software, firmware, and/or hardware. Thus, one or more of the techniques shown in the Figures (e.g., flowcharts) may be implemented using code and data stored and executed on one or more electronic devices or nodes (e.g., a subscriber client device or end station, a network element, etc.). Such electronic devices may store and communicate (internally and/or with other electronic devices over a network) code and data using computer-readable media, such as non-transitory computer-readable storage media (e.g., magnetic disks, optical disks, random access memory, read-only memory, flash memory devices, phase-change memory, etc.), transitory computer-readable transmission media (e.g., electrical, optical, acoustical or other form of propagated signals-such as carrier waves, infrared signals, digital signals), etc. In addition, such network elements may typically include a set of one or more processors coupled to one or more other components, such as one or more storage devices (e.g., non-transitory machine-readable storage media) as well as storage database(s), user input/output devices (e.g., a keyboard, a touch screen, a pointing device, and/or a display), and network connections for effectuating signaling and/or bearer media transmission.

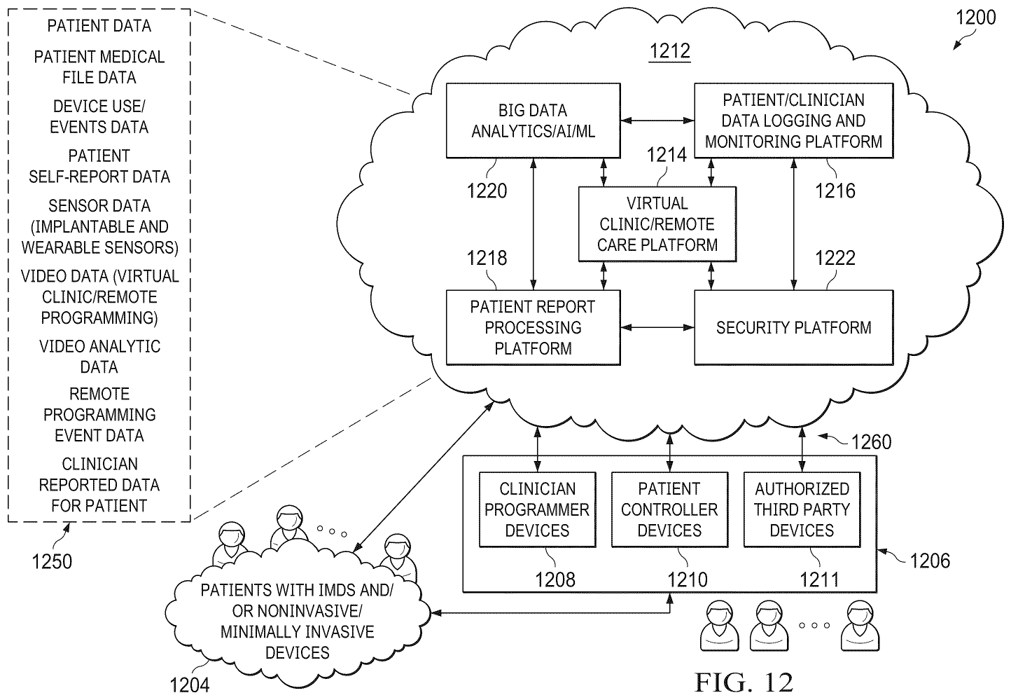

Without limitation, an example cloud-centric digital healthcare network architecture involving various network-based components, subsystems, service nodes etc., as well as myriad end user deployments concerning patients, clinicians and authorized third-party agents/users is illustrated in FIG. 12 wherein some example embodiments of the present patent disclosure may be practiced. In one arrangement, example architecture 1260 may include one or more virtual clinic (VC) platforms 1214, remote data logging platforms 1216, patient/clinician report processing platforms 1218, as well as data analytics platforms 1220 and security platforms 1222, at least some of which may be configured and/or deployed as at least a part of an integrated digital health infrastructure 1212 for effectuating some example embodiments of the present disclosure. One or more pools of patients having a myriad of health conditions and/or receiving assorted treatments, who may be geographically distributed in various locations, areas, regions, etc., are collectively shown at reference numeral 1204, wherein individual patients may be provided with one or more suitable IMDs/IPGs, NIMI devices, other personal biomedical instrumentation, etc., depending on respective patients' health conditions and/or treatments. A plurality of clinician programmer (CP) devices 1208, patient controller (PC) devices 1210, and authorized third-party devices 1211 associated with respective users (e.g., clinicians, medical professionals, patients and authorized agents thereof such as family members, caregivers, etc. may be deployed as external devices 1206 that may be configured to interact with patients' IMDs and/or NIMI devices for effectuating therapy, monitoring, data logging, secure file transfer, etc., via local communication paths or over network-based remote communication paths established in conjunction with the digital health infrastructure network 1212.

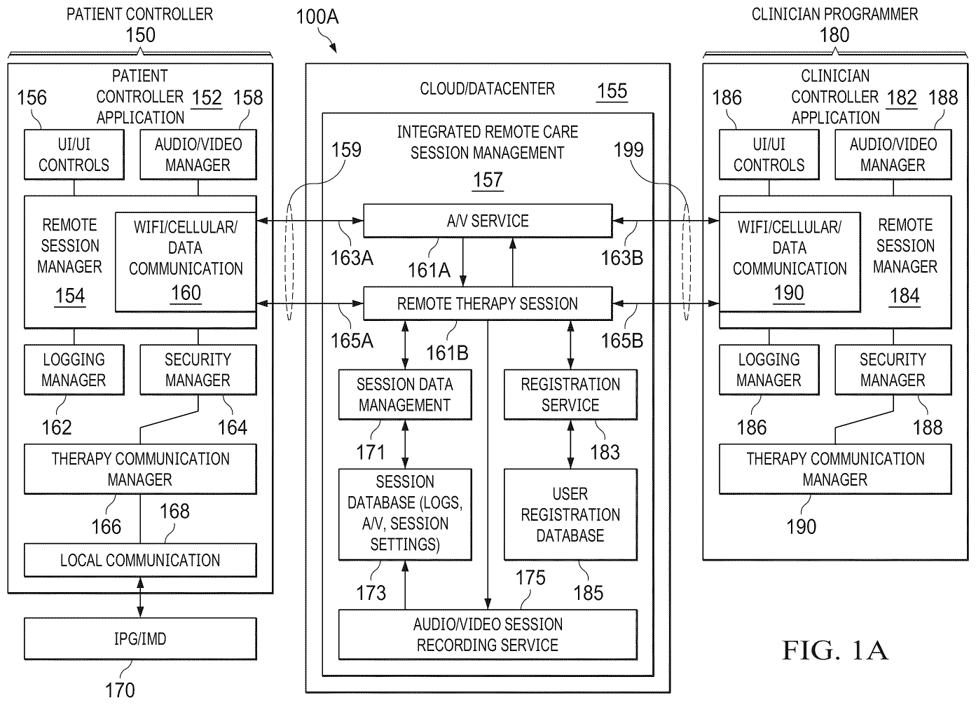

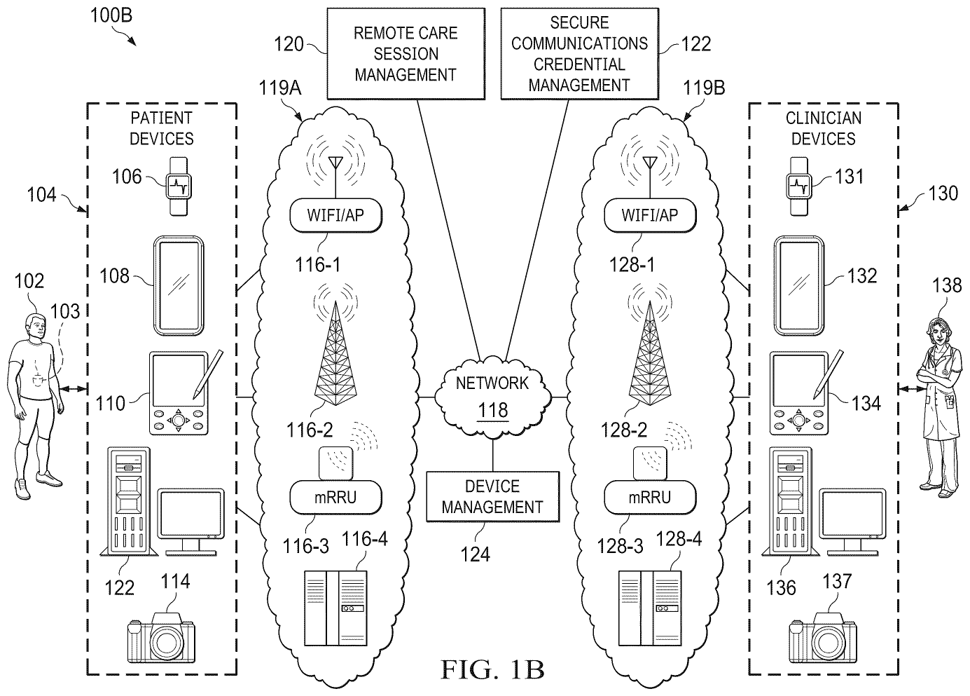

In one arrangement, example architecture 1260 may encompass a hierarchical/heterogeneous network arrangement comprised of one or more fronthaul radio access network (RAN) portions or layers, one or more backhaul portions or layers, and one or more core network portions or layers, each of which may in turn include appropriate telecommunications infrastructure elements, components, etc., cooperatively configured for effectuating a digital healthcare ecosystem involving patients' IMDs and/or NIMI devices 1204, external devices 1206, and one or more components of the digital health infrastructure network 1212, wherein at least a portion of the components of the infrastructure network 1212 may be operative as a cloud-based system for purposes of some embodiments herein. Further, at least a portion of the components of the digital health infrastructure network 1212 operating as a system 1200, one or more patients' IMDs and/or NIMI devices 1204, and one or more external devices 1206 (including, e.g., third-party devices) may be configured to execute suitable medical/health software applications in a cooperative fashion, e.g., in a server-client relationship, facilitated by VC platform 1214 for effectuating various aspects of remote patient monitoring, telemedicine/telehealth applications, remote care therapy, A/V session redirection/transfer between endpoints, enablement of one or more third parties to join an A/V session of an ongoing remote care therapy session, enforcement privacy policy controls, remote assistance, etc. In some arrangements, VC platform 1214 may therefore be configured with components and functionalities associated with remote care session management (RCSM) 157 shown in FIG. 1A and/or subsystems 120 and 122 as well as device management 124 (e.g., enterprise device management system (EDMS), also referred to as a mobile/medical device management system (MDMS), user profile management, etc.) exemplified in FIG. 1B, as will be set forth further below.

In some example arrangements, a virtual clinic may be configured to provide patients and/or clinicians the ability to perform remote therapies using a secure telehealth session. To enhance clinician interaction and evaluation of a patient during a secure telehealth session, example embodiments herein may be configured to provide various user interface (UI) layouts and controls for clinician programmer devices and/or patient controller devices for facilitating real-time kinematic and/or auditory data analysis, which may be augmented with suitable artificial intelligence (AI) and/or machine learning (ML) techniques (e.g., neural networks, etc.) in some arrangements. AI/ML techniques may also be implemented in some arrangements that may involve image blurring and anonymization pursuant to privacy policy control according to some embodiments. Further, some example embodiments with respect to these aspects may involve providing kinematic UI settings that enable different types of overlays, e.g., with or without a pictorial representation of the patient. Some example embodiments may be configured to enable one or more of the following features and functionalities: (i) separate or combined audio and/or peripheral sensor streams; (ii) capture of assessments from separate or different combinations of body features such as, e.g., limbs, hands, face, etc.; (iii) replay of another clinician's video including the patient's kinematic analysis (e.g., a secondary video stream with patient data), and the like.

Additional details with respect to the various constituent components of the digital health infrastructure 1212, example external devices 1206 comprising clinician programmer devices 1208, patient controller devices 1210 and/or third-party devices 1211, as well as various interactions involving the network-based entities and the end points (also referred to as edge devices) will be set forth immediately below in order to provide an example architectural framework wherein one or more embodiments may be implemented and/or augmented according to the teachings herein.

Turning to FIG. 1A, depicted therein is an example architecture of a system configured to support remote patient therapy as part of an integrated remote care service session in a virtual clinic environment that may be deployed in a cloud-centric digital health implementation for purposes of an embodiment of the present patent disclosure. As used herein, a “remote care system” may describe a healthcare delivery system configured to support a remote care service over a network in a communication session between a patient and a clinician wherein telehealth or telemedicine applications involving remote medical consultations as well as therapy applications involving remote programming of the patient's IMD may be launched via a unified application interface facilitated by one or more network entities (e.g., as a virtual clinic platform). In some arrangements, a remote care system may also include a remote patient monitoring system and/or a remote healthcare provisioning system without the involvement of a clinician. In still further arrangements, a remote care system may include one or more AI-based expert systems or agents, e.g., involving supervised learning, that may be deployed in a network to provide or otherwise augment the capabilities of a system to effectuate enhanced healthcare solutions relating to diagnosis, remote learning, therapy selection, as well as facilitate network-based solutions for enhancing patients' overall well-being. In some aspects, remote care therapy may involve any care, programming, or therapy instructions that may be provided by a doctor, a medical professional or a healthcare provider, and/or their respective authorized agents, collectively referred to as a “clinician”, using a suitable clinician device, with respect to the patient's IMD/NIMI device, wherein such therapy instructions may be mediated, proxied or otherwise relayed by way of a controller device associated with the patient. As illustrated, example remote care system 100A may include a plurality of patient controller devices exemplified by patient controller device 150 and a plurality of clinician programmer devices exemplified by a clinician programmer device 180 (also referred to as a clinician programmer or clinician device) that may interact with a network-based infrastructure via respective communication interfaces. Example patient and clinician devices may each include a corresponding remote care service application module, e.g., a patient controller application 152 and a clinician programmer/controller application 182, executed on a suitable hardware/software platform for supporting a remote care service that may be managed by a network entity 155. In some embodiments, example network entity 155 may comprise a distributed datacenter or cloud-based service infrastructure (e.g., disposed in a public cloud, a private cloud, or a hybrid cloud, involving at least a portion of the Internet) operative to host a remote care session management (RCSM) service 157. In one arrangement, patient controller application 152 and clinician programmer application 182 may each include a respective remote session manager 154, 184 configured to effectuate or otherwise support a corresponding communication interface 160, 190 with network entity 155 using any known or heretofore unknown communication protocols and/or technologies. In one arrangement, interfaces 160, 190 are each operative to support an audio/video (A/V) or audiovisual (AV) channel or session 163A, 163B and a remote therapy channel or session 165A, 165B, respectively, with an AV communication service 161A and a remote therapy session service 161B of the remote care session management service 157 as part of a common bi-directional remote care session 159, 199 established therewith. In one arrangement, patient controller application 152 and clinician programmer application 182 may each further include or otherwise support suitable graphical user interfaces (GUIs) and associated controls 156, 186, as well as corresponding AV managers 158, 188, each of which may be interfaced with respective remote session managers 154, 184 for purposes of one or more embodiments of the present disclosure as will be set forth in additional detail further below. Remote care session manager 154 of the patient controller application 152 and remote care session manager 184 of the clinician programmer application 182 may each also be interfaced with a corresponding data logging manager 162, 186 for purposes of still further embodiments of the present disclosure. In one arrangement, remote care session manager 154 of patient controller application 152 is further interfaced with a security manager 164, which may be configured to facilitate secure or trusted communication relationships with the network entity 155. Likewise, remote care session manager 184 of clinician programmer application 182 may also be interfaced with a security manager 188 that may be configured to facilitate secure or trusted communication relationships with the network entity 155. Each security manager 164, 188 may be interfaced with a corresponding therapy communication manager 166, 190 with respect to facilitating secure therapy communications between the clinician programmer device 180 and the patient controller device 150. Therapy communication manager 166 of the patient controller application 152 may also interface with a local communication module 168 operative to effectuate secure communications with the patient's IPG/IMD 170 using a suitable short-range communications technology or protocol. In still further arrangements, security managers 164, 188 of patient controller and clinician programmer applications 152, 182 may be configured to interface with the remote care session management service 157 to establish trusted relationships between patient controller device 150, clinician programmer device 180 and IPG/IMD 170 based on the exchange of a variety of parameters, e.g., trusted indicia, cryptographic keys and credentials, etc.

In one arrangement, the integrated remote care session management service 157 may include a session data management module 171, an AV session recording service module 175 and a registration service module 183, as well as suitable database modules 173, 185 for storing session data and user registration data, respectively. In some arrangements, at least part of the session data may include user-characterized data relating to AV data, therapy settings data, network contextual data, and the like, for purposes of still further embodiments of the present patent disclosure.

Skilled artisans will realize that example remote care system architecture 100A set forth above may be advantageously configured to provide both telehealth medical consultations as well as therapy instructions over a communications network while the patient and the clinician/provider are not in close proximity of each other (e.g., not engaged in an in-person office visit or consultation). Accordingly, in some embodiments, a remote care service of the present disclosure may form an integrated healthcare delivery service effectuated via a common application user interface that not only allows healthcare professionals to use electronic A/V communications to evaluate and diagnose patients remotely but also facilitates remote programming of the patient's IPG/IMD for providing appropriate therapy, thereby enhancing efficiency as well as scalability of a delivery model. Additionally, example remote care system architecture 100A may be configured to effectuate various other aspects relating to enhanced functionalities such as, e.g., remote assistance, enablement of third parties, privacy controls, A/V session redirection, etc., which will be set forth in additional detail hereinbelow. Further, an implementation of example remote care system architecture 100A may involve various types of network environments deployed over varying coverage areas, e.g., homogenous networks, heterogeneous networks, hybrid networks, etc., which may be configured or otherwise leveraged to provide patients with relatively quick and convenient access to diversified medical expertise that may be geographically distributed over large areas or regions, preferably via secure communications channels for purposes of at least some example embodiments of the present disclosure.

FIG. 1B depicts an example network environment 100B wherein the remote care service architecture of FIG. 1A may be implemented according to some embodiments. Illustratively, example network environment 100B may comprise any combination or sub-combination of a public packet-switched network infrastructure (e.g., the Internet or worldwide web, also sometimes referred to as the “cloud”, as noted above), private packet-switched network infrastructures such as Intranets and enterprise networks, health service provider network infrastructures, and the like, any of which may span or involve a variety of access networks, backhaul and core networks in an end-to-end network architecture arrangement between one or more patients, e.g., patient(s) 102, and one or more authorized clinicians, healthcare professionals, or agents thereof, e.g., generally represented as caregiver(s) or clinician(s) 138. Example patient(s) 102, each having one or more suitable implantable and/or NIMI devices, e.g., generally or cumulatively represented as IMD 103, may be provided with a variety of corresponding external devices for controlling, programming, otherwise (re) configuring the functionality of respective implantable device(s) 103, as is known in the art. Such external devices associated with patient(s) 102, referred to herein as patient devices 104, which are representative of patient controller device 150 shown in FIG. 1A, may comprise a variety of user equipment (UE) devices, tethered or untethered, that may be configured to engage in remote care sessions involving telehealth and/or therapy sessions according to some embodiments described below. By way of example, patient devices 104 may comprise commercial off-the-shelf (COTS) equipment or proprietary portable medical/healthcare devices (non-COTS), which may be configured to execute a therapy/digital healthcare application program or “app”, wherein various types of communications relating to control, therapy/diagnostics, and/or device file management may be effectuated for purposes of some embodiments. Accordingly, example patient devices 104 may include, in addition to proprietary medical devices, devices such as smartphones, tablets or phablets, laptops/desktops, handheld/palmtop computers, wearable devices such as smart glasses and smart watches, personal digital assistant (PDA) devices, smart digital assistant devices, etc., any of which may operate in association with one or more virtual assistants, smart home/office appliances, smart TVs, external/auxiliary AV equipment, virtual reality (VR), mixed reality (MR) or augmented reality (AR) devices, and the like, which are generally exemplified by wearable device(s) 106, smartphone(s) 108, tablet(s)/phablet(s) 110, computer(s) 112, and AV equipment 114. As such, example patient devices 104 may include various types of communications circuitry or interfaces to effectuate wired or wireless communications, short-range and long-range radio frequency (RF) communications, magnetic field communications, etc., using any combination of technologies, protocols, and the like, with external networked elements and/or respective implantable devices 103 corresponding to patient(s) 102. In still further arrangements, patient devices 104 may also include one or several devices or A/V equipment operative as auxiliary/secondary devices or endpoints to which an A/V session may be redirected according to some embodiments herein. As such, a device configured to operate as an auxiliary/secondary device endpoint may comprise any of the foregoing devices, wherein such a device may have a higher resolution or larger screen than a device used as a primary patient controller device, e.g., for purposes of providing a higher quality video display when an A/V session is redirected thereto according to the embodiments of the present disclosure. With respect to networked communications, patient devices 104 may be configured, independently or in association with one or more digital/virtual assistants, smart home/premises appliances and/or home networks, to effectuate mobile communications using technologies such as Global System for Mobile Communications (GSM) radio access network (GRAN) technology, Enhanced Data Rates for Global System for Mobile Communications (GSM) Evolution (EDGE) network (GERAN) technology, 4G Long Term Evolution (LTE) technology, Fixed Wireless technology, 5th Generation Partnership Project (5GPP or 5G) technology, Integrated Digital Enhanced Network (IDEN) technology, WiMAX technology, various flavors of Code Division Multiple Access (CDMA) technology, heterogeneous access network technology, Universal Mobile Telecommunications System (UMTS) technology, Universal Terrestrial Radio Access Network (UTRAN) technology, All-IP Next Generation Network (NGN) technology, as well as technologies based on various flavors of IEEE 802.11 protocols (e.g., WiFi), and other access point (AP)-based technologies and microcell-based technologies involving small cells, femtocells, picocells, etc. Further, some embodiments of patient devices 104 may also include interface circuitry for effectuating network connectivity via satellite communications. Where tethered UE devices are provided as patient devices 104, networked communications may also involve broadband edge network infrastructures based on various flavors of Digital Subscriber Line (DSL) architectures and/or Data Over Cable Service Interface Specification (DOCSIS)-compliant Cable Modem Termination System (CMTS) network architectures (e.g., involving hybrid fiber-coaxial (HFC) physical connectivity). Accordingly, by way of illustration, an edge/access network portion 119A is exemplified with elements such as WiFi/AP node(s) 116-1, macro-cell node(s) 116-2 such as eNB nodes, gNB nodes, etc., microcell nodes 116-3 (e.g., including micro remote radio units or RRUs, etc.) and DSL/CMTS node(s) 116-4.

In similar fashion, clinicians and/or clinician agents 138 may be provided with a variety of external devices for controlling, programming, otherwise (re) configuring or providing therapy operations with respect to one or more patients 102 mediated via respective implantable device(s) 103, in a local therapy session and/or telehealth/remote therapy session, depending on implementation and use case scenarios. External devices associated with clinicians/agents 138, referred to herein as clinician devices 130, which are representative of clinician programmer device 180 shown in FIG. 1A, may comprise a variety of UE devices, tethered or untethered, similar to patient devices 104, that may be configured to engage in telehealth and/or remote care therapy sessions as will be set forth in detail further below. Clinician devices 130 may therefore also include non-COTS devices as well as COTS devices, generally exemplified by wearable device(s) 131, smartphone(s) 132, tablet(s)/phablet(s) 134, computer(s) 136 and external/auxiliary AV equipment 137, any of which may operate in association with one or more virtual assistants, smart home/office appliances, VR/AR/MR devices, and the like. Further, example clinician devices 130 may also include various types of network communications circuitry or interfaces similar to that of personal devices 104, which may be configured to operate with a broad range of technologies as set forth above. Further, example clinician devices 130 may also include devices operative as auxiliary/secondary devices to which an A/V session terminating at a primary clinician programmer device may be redirected/transferred to otherwise provided to, similar to the auxiliary/secondary devices associated with the patients, which may be connected to a healthcare network using a variety of technologies as previously noted. Accordingly, an edge/access network portion 119B is exemplified as having elements such as WiFi/AP node(s) 128-1, macro/microcell node(s) 128-2 and 128-3 (e.g., including micro remote radio units or RRUs, base stations, eNB/gNB nodes, etc.) and DSL/CMTS node(s) 128-4. It should therefore be appreciated that edge/access network portions 119A, 119B may include all or any subset of wireless/wireline communication infrastructures, technologies and protocols for effectuating data communications with respect to some embodiments of the present disclosure.

In one arrangement, a plurality of network elements or nodes may be provided for facilitating an integrated remote care therapy service involving one or more clinicians 138 and one or more patients 102, wherein such elements are hosted or otherwise operated by various stakeholders in a service deployment scenario depending on implementation, e.g., including one or more public clouds, private clouds, or any combination thereof as previously noted. According to some example embodiments, a remote care session management node or platform 120 may be provided, generally representative of the network entity 157 shown in FIG. 1A, preferably disposed as a cloud-based element coupled to network 118, that is operative in association with a secure communications credentials management node 122 and a device management node 124, to facilitate a virtual clinic platform whereby a clinician may advantageously engage in a telehealth session and/or a remote care therapy session with a particular patient via a common application interface and associated AV and therapy controls, as will be described further below.

It should be appreciated that although example network environment 100B does not specifically show third-party devices operated by authorized agents/users of the patients and/or clinicians, such devices having a suitable application program executing thereon, albeit with lesser authorization levels and functionalities, may be deployed in an arrangement for purposes of some embodiments herein. Further, such third-party devices may comprise COTS and/or non-COTS devices, similar to patient devices 104 and/or clinician devices 130, depending on implementation.

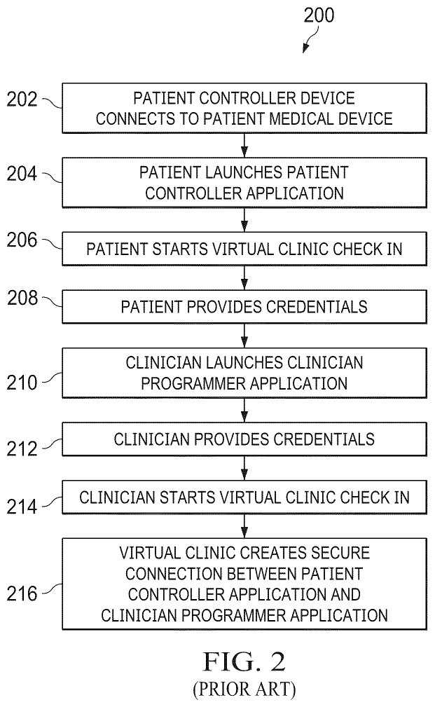

FIG. 2 depicts a flowchart for establishing a remote programming or virtual clinic session according to known processes. Additional details regarding establishment of remote programming or virtual clinic sessions may be found in U.S. Pat. No. 10,124,177 which is incorporated herein by reference. Although some details are described herein regarding establishment of a virtual clinic/remote programming session, any suitable methods may be employed according to other embodiments. At block 202, the patent controller device connects to the patient's medical device. For example, the patent controller device may establish a BLUETOOTH communication session with the patient's implantable pulse generator. At block 204, the patient launches the patient controller app on the patient controller device. At block 206, the patient starts a virtual clinic check in process by selecting a suitable GUI component of the patient controller app. In block 208, the patient may provide patient credentials. At block 210, the clinician launches the clinician programmer app on the clinician programmer device. In block 212, the clinician provides credentials. At block 214, the clinician checks into the virtual clinic to communicate with the patient. At block 216, the virtual clinic infrastructure establishes a secure connection between the patient controller app and clinician programmer app to conduct communications. Known cybersecurity features may be applied to establish the secure connection including using Public Key Infrastructure (PKI) processes, encryption processes, authentication processes, etc. Biometric data and other data may also be employed to enhance the secure nature of the communication session by validating user identities and authorization. Upon establishment, the communications may include audio and video communications between the patient and the clinician. Also, the clinician may conduct remote programming of the patient's medical device during the session while communicating with the patient.

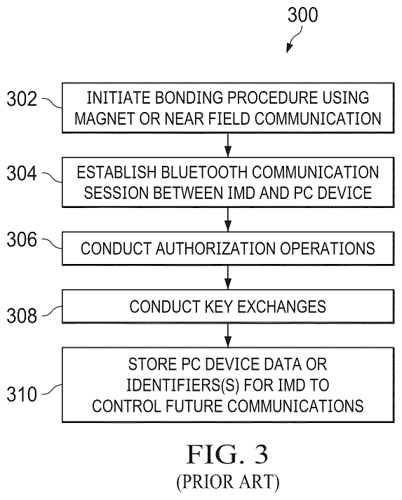

FIG. 3 depicts a flowchart illustrative of known blocks, steps and/or acts that may be implemented for establishing a communication session with an implantable medical device. Additional details regarding establishing a communication session with an implantable medical device may be found in U.S. Pat. No. 11,007,370 which is incorporated herein by reference. Although example operations are described in FIG. 3, any suitable methods of securing communication between a patient controller (PC) device and a patient medical device may be employed as appropriate for a given patient therapy. At block 302, a bonding procedure is initiated to establish a trusted relationship between the implantable medical device or other medical device of the patient and a patient controller device. The bonding procedure may be initiated by using a magnet to activate a Hall sensor in the medical device. Alternatively, near field communication (e.g., inductive coupling) may be employed to initiate the bonding procedure. The use of a magnetic or inductive coupling provides a degree of physical access control to limit the possibility of unauthorized devices from communicating with the patient's medical device. That is, a device that attempts to obtain authorization to communicate must be brought into physical proximity with the patient at a time that is controlled by the patient thereby reducing the possibility of unauthorized devices from improperly gaining the ability to communicate with the patient's device. At block 304, a communication session is established between the patient's medical device and the patient's controller (PC) device. For example, a BLUETOOTH communication session may be established. At block 306, authentication operations are conducted. For example, known credentials, PKI, encryption, and other cybersecurity operations may be applied between the patient's medical device and the PC device to determine whether the PC device should be allowed to conduct communications with the patient's medical device. At block 308, encryption key data may be exchanged between devices for future communications. At block 310, other PC identifiers or data may be stored in IMD to control future communications. Upon establishment of a trusted relationship between the patient's medical device and a PC device, the PC device may be used to conduct remote programming session. The remote programming sessions may also be subjected to cybersecurity methods such as the use of credentials, PKI, encryption, etc.

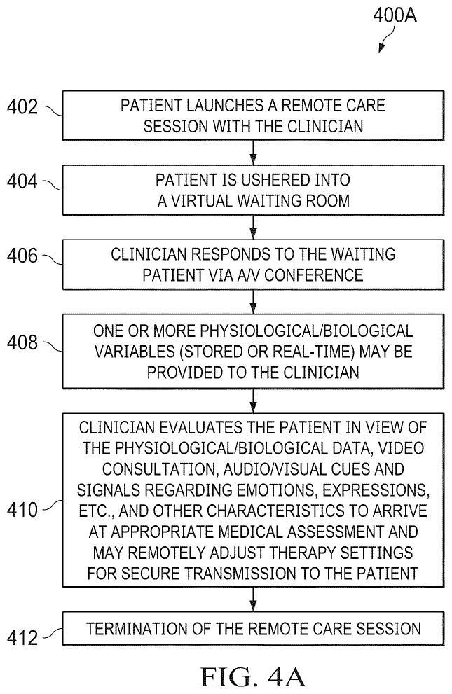

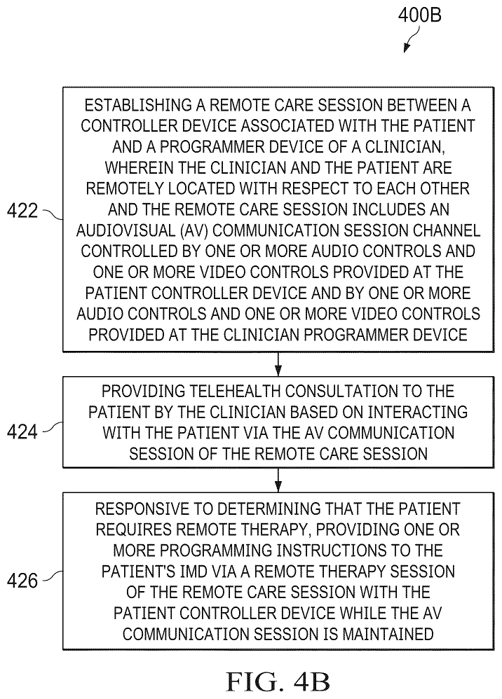

FIGS. 4A and 4B depicts flowcharts illustrative of a remote care scenario involving an example digital health network architecture wherein an integrated remote care session may be established between a patient and a clinician operating respective controller devices that support suitable graphical user interfaces (GUIs) for facilitating a therapy session, an audio/visual (AV) communication session, or a combination of both, for purposes of some example embodiments of the present disclosure. As will be set forth further below, patient controller and/or clinician programmer devices may be provided with appropriate application software to effectuate suitable GUIs on respective devices for facilitating a remote care session including a secure AV session/channel and a therapy session/channel as part of a common application interface that can support telehealth/telemedicine applications, remote monitoring, remote therapy, remote assistance, data logging, privacy policy control, A/V session redirection, third-party enablement, etc. Process flow 400A of FIG. 4A may commence with a patient launching an integrated digital health application executing on the patient controller/device to initiate a secure communications channel with a remote clinician (block 402), e.g., by selecting a “Remote Care” option from a pull-down menu, clicking on an icon on the UI display screen, or via a voice command, etc. In one embodiment, the patient may be ushered into a virtual waiting room, which may be realized in a UI screen window of the patient/clinician device (block 404). At block 406, the clinician responds to the waiting patient, e.g., via a secure AV communication channel of the remote care session. At block 408, one or more physiological/biological data of the patient (stored or real-time) may be provided to the clinician via secure communications. In some embodiments, one or more digital keys of the clinician and/or the patient may be employed to secure communications. At block 410, the clinician evaluates the patient in view of the physiological/biological data, telemedicine/video consultation, audio/visual cues and signals regarding patient's facial expressions, hand movement/tremors, walking, gait, ambulatory status/stability, and other characteristics to arrive at appropriate medical assessment. Depending on such telehealth consultation/evaluation, the clinician may remotely adjust stimulation therapy settings for secure transmission to the patient device, which may be securely transmitted via encrypted communications. In a further scenario, a remote clinician proxy or agent may be executed at or in association with the patient controller/device upon launching a remote session, wherein the proxy/agent is operative to effectuate or otherwise mediate the transmission of any therapy settings to the patient's IMD, either in real-time or at some point in the future depending upon programmatic control. After completing the requisite therapy and consultative communications, the remote care session may be terminated, e.g., either by the clinician and/or the patient, as set forth at block 412.

Process flow 400B of FIG. 4B is illustrative of an embodiment of a high level scheme for delivering healthcare to a patient via an integrated remote care session. At block 422, a remote care session between a controller device associated with the patient and a programmer device associated with a clinician may be established, wherein the clinician and the patient are remotely located with respect to each other and the remote care session includes an AV communication session controlled by one or more A/V controls provided at the patient controller device and the clinician programmer device. At block 424, various telehealth consultation services may be provided to the patient by the clinician based on interacting with the patient via the AV communication channel of the remote care session as previously noted. Responsive to determining that the patient requires remote therapy, one or more remote programming instructions may be provided to the patient's IMD via a remote therapy session or channel of the remote care session with the patient controller device while the AV communication session is maintained (block 426).

Skilled artisans will recognize that some of the blocks, steps and/or acts set forth above may take place at different entities and/or different times (i.e., asynchronously), and possibly with intervening gaps of time and/or at different locations. Further, some of the foregoing blocks, steps and/or acts may be executed as a process involving just a single entity (e.g., a patient controller device, a clinician programmer device, or a remote session manager operating as a virtual clinic, etc.), or multiple entities, e.g., as a cooperative interaction among any combination of the end point devices and the network entities. Still further, it should be appreciated that example process flows may be interleaved with one or more sub-processes comprising other IMD<=>patient or IMD<=>clinician interactions (e.g., local therapy sessions) as well as virtual clinic<=>patient or virtual clinic<=>clinician interactions (e.g., remote patient monitoring, patient/clinician data logging, A/V session transfer between endpoints, third-party enablement, remote assistance, etc., as will be set forth further below). Accordingly, skilled artisans will recognize that example process flows may be altered, modified, augmented or otherwise reconfigured for purposes of some embodiments herein.

In one implementation, an example remote care session may be established between the patient controller device and the clinician programmer device after the patient has activated a suitable GUI control provided as part of a GUI associated with the patient controller device and the clinician has activated a corresponding GUI control provided as part of a virtual waiting room displayed on a GUI associated with the clinician programmer device. In another arrangement, remote programming instructions may be provided to the patient's IMD via the remote therapy session only after verifying that remote care therapy programming with the patient's IMD is compliant with regulatory requirements of one or more applicable local, regional, national, supranational governmental bodies, non-governmental agencies, and international health organizations. In a still further variation, various levels of remote control of a patient's controller and its hardware by a clinician programmer device may be provided. For example, suitable GUI controls may be provided at the clinician programmer device for remotely controlling a camera component or an auxiliary AV device associated with the patient controller device by interacting with a display of the patient's image on the screen of the clinician programmer device, e.g., by pinching, swiping, etc., to pan to and/or zoom on different parts of the patient in order to obtain high resolution images. Additional embodiments and/or further details regarding some of the foregoing variations with respect to providing remote care therapy via a virtual clinic may be found in the following U.S. patent applications, publications and/or patents: (i) U.S. Patent Application Publication No. 2020/0398062, entitled “SYSTEM, METHOD AND ARCHITECTURE FOR FACILITATING REMOTE PATIENT CARE”; (ii) U.S. Patent Application Publication No. 2020/0402656, entitled “UI DESIGN FOR PATIENT AND CLINICIAN CONTROLLER DEVICES OPERATIVE IN A REMOTE CARE ARCHITECTURE”; (iii) U.S. Patent Application Publication No. 2020/0402674, entitled “SYSTEM AND METHOD FOR MODULATING THERAPY IN A REMOTE CARE ARCHITECTURE”; and (iv) U.S. Patent Application Publication No. 2020/0398063, entitled “DATA LABELING SYSTEM AND METHOD OPERATIVE WITH PATIENT AND CLINICIAN CONTROLLER DEVICES DISPOSED IN A REMOTE CARE ARCHITECTURE”, each of which is hereby incorporated by reference herein.

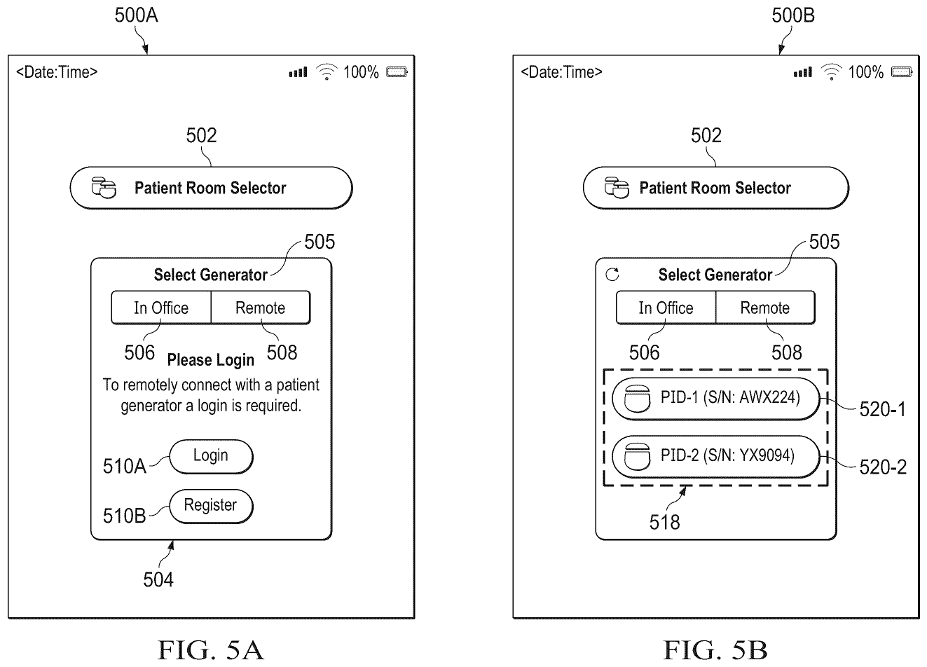

FIGS. 5A and 5B depict representations of an example user interface and associated dialog boxes or windows provided with a clinician programmer device, e.g., as a touch screen display of device 180 exemplified in FIG. 1A, for selecting different therapy applications and/or service modes in an integrated remote care service application for purposes of some example embodiments of the present disclosure. In one arrangement, example GUI(s) of the clinician device may be optimized or resized to provide a maximum display window for the presentation of a patient's image during remote therapy while allowing the presentation of appropriate remote care therapy session and setting controls as well as AV communication session controls such that high quality video/image information may be advantageously obtained by the clinician, which can help better evaluate the patient's response(s) to the applied/modified therapy settings and/or the clinician's verbal, textual, and/or visual requests to perform certain tasks as part of remote monitoring by the clinician. Accordingly, in some example embodiments, the clinician device may be provided with one or more non-transitory tangible computer-readable media or modules having program code stored thereon for execution on the clinician device as part of or in association with a clinician programmer application for facilitating remote therapy and telehealth delivery in an integrated session having a common application interface that effectuates an optimized GUI display within the form factor constraints of the device. In one arrangement, a code portion may be provided for displaying a virtual waiting room identifying one or more patients, each having at least one IMD/NIMI device configured to facilitate a therapy, wherein the virtual waiting room is operative to accept input by the clinician to select a patient to engage in a remote care session with a patient controller device of the selected patient. A code portion may be provided for displaying one or more audio controls and one or more video controls for facilitating an AV communication session associated with the remote care session after the remote care session is established between the patient controller device and the clinician programmer device. Various AV session controls may be represented as suitable icons, pictograms, etc. as part of a GUI display of the at the clinician programmer device, roughly similar to the GUI presentation at a patient controller device as will be set forth below. Further, example video controls may be configured to effectuate a first display window (i.e., a clinician image window) and a second display window (i.e., a patient image window) on the GUI display for respectively presenting an image of the clinician and an image of the patient. A code portion may be provided for displaying one or more remote care therapy session and setting controls, wherein the one or more remote care therapy setting controls are operative to facilitate one or more adjustments with respect to the patient's IMD settings in order to provide appropriate therapy to the patient as part of a remote therapy component of the remote care session. Preferably, the code portion may be configured to provide the AV communication session controls as well as the remote care therapy session and setting controls in a consolidated manner so as to facilitate the display thereof in a minimized overlay panel presented on the GUI screen while maximizing the second display window such that an enlarged presentation of the patient's image is effectuated during the remote care session. In some embodiments, the remote care therapy setting controls may be configured to expand into additional graphical controls for further refining one or more IMD settings depending on the implementation and/or type(s) of therapy applications the clinician programmer device is configured with. For example, such remote care therapy setting controls may comprise icons or pictograms corresponding to, without limitation, one or more of a pulse amplitude setting control, a pulse width setting control, a pulse frequency setting control, a pulse delay control, a pulse repetition parameter setting control, a biphasic pulse selection control, a monophasic pulse section control, a tonic stimulation selection control, a burst stimulation selection control, a lead selection control, an electrode selection control, and a “Stop Stimulation” control, etc., at least some of which may be presented in a set of hierarchical or nested pull-down menus or display windows. In still further embodiments, a code portion may be provided for displaying one or more data labeling buttons as part of the GUI display of the clinician programmer device, similar to the GUI embodiments of the patient controller device described above, wherein the one or more data labeling buttons are operative to accept input by the clinician corresponding to a subjective characterization of AV quality, therapy response capture, and other aspects of therapy programming during the remote care session. In still further embodiments, one or more code portions may be provided for displaying appropriate GUI controls, icons, dialog boxes, etc. configured to facilitate functionalities such as, e.g., A/V redirection, privacy policy control, third-party enablement, and the like.

GUI screen 500A depicted in FIG. 5A is representative of a “login” screen that may be presented at the clinician device upon launching the clinician programmer application for facilitating a clinician to select a service mode, e.g., a remote care service mode or an in-office care service mode. A “Patient Room” selector menu option 502 may be operative to present a “generator” window 505 that includes an “In-Office” patient option 506 or a “Remote” patient option 508, wherein the activation or selection of the Remote patient option 508 effectuates one or more windows or dialog boxes for facilitating user login, registration, authentication/authorization and other security credentialing services, as exemplified by windows 510A, 510B. Upon validation, the clinician may be presented with a virtual waiting room 518 identifying one or more remote patients as exemplified in GUI screen 500B of FIG. 5B. Each remote patient may be identified by one or more identifiers and/or indicia, including, without limitation, personal identifiers, respective IMD identifiers, therapy identifiers, etc., subject to applicable privacy and healthcare laws, statutes, regulations, and the like. Accordingly, in some embodiments such identification indicia may comprise, inter alia, patient names, images, thumbnail photos, IMD serial numbers, etc., collectively referred to as Patient ID (PID) information, as illustrated by PID-1 520-1 and PID-2 520-2. In some embodiments, a time indicator may be associated with each remote patient, indicating how long a remote patient has been “waiting” (e.g., the time elapsed since launching a remote care session from his/her controller device). In some embodiments, a priority indicator may also be associated with remote patients, wherein different priorities may be assigned by an intervening human and/or AI/ML-based digital agent. Furthermore, patients may have different types of IMDs to effectuate different therapies and a patient may have more than one IMD in some cases. An example embodiment of virtual waiting room 518 may therefore include a display of any combination of remote patients and their respective IMDs by way of suitably distinguishable PIDs having various pieces of information, wherein the PIDs may be individually selectable by the clinician for establishing a remote care session that may include remote therapy programming or just telehealth consultations.

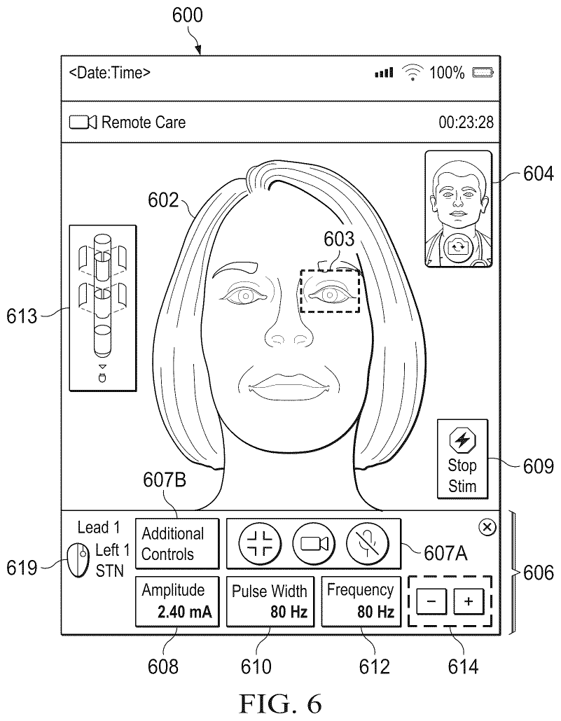

FIG. 6 depicts a representation of an example user interface of a clinician programmer device with additional details for facilitating graphic controls with respect to an AV communication session and a remote therapy session in a remote care service application for effectuating an integrated remote programming session and associated AV communication session for purposes of some embodiments of the present disclosure. As illustrated, GUI screen 600 is representative of a display screen that may be presented at the clinician device after establishing that remote therapy programming is to be effectuated for a selected remote patient. In accordance with some of the embodiments set forth herein, GUI screen 600 may be arranged so that the patient's video image is presented in an optimized or resized/oversized display window 602 while the clinician's video image is presented in a smaller display window 604 along with a compact control icon panel 606 to maximize the level of detail/resolution obtained in the patient's image. Furthermore, the smaller clinician image window 604 may be moved around the UI screen by “dragging” the image around the viewing area of the patient's image window 602 to allow more control of the positioning of the display windows so that the patient's image view is unimpeded and/or optimized at a highest possible resolution. It will be appreciated that such high level video quality is particularly advantageous in obtaining more reliable cues with respect to the patient's facial expressions, moods, gestures, eye/iris movements, lip movements, hand movements tremors, jerks, twitches, spasms, contractions, or gait, etc., that may be useful in diagnosing various types of motor/neurological disorders, e.g., Parkinson's disease. In some further arrangements, a remote data logging platform may be configured to store the AV data of the sessions for facilitating model building and training by appropriate AI/ML-based expert systems or digital assistants for purposes of further embodiments of the present patent disclosure.

Control panel window 606 may include a sub-panel of icons for AV and/or remote care session controls, e.g., as exemplified by sub-panel 607A in addition to a plurality of icons representing remote therapy setting controls, e.g., pulse amplitude control 608, pulse width control 610, pulse frequency control 612, increment/decrement control 614 that may be used in conjunction with one or more therapy setting controls, along with a lead selection indication icon 619. In some example embodiments, additional control buttons, icons, etc. collectively shown at reference numeral 607B, may be provided as part of control panel window 606 for facilitating AV endpoint transfer, permission to enable third parties, setting of privacy controls, etc. Skilled artisans will recognize that the exact manner in which a control panel window may be arranged as part of a consolidated GUI display depends on the therapy application, IMD deployment (e.g., the number of leads, electrodes per lead, electrode configuration, etc.), and the like, as well as the particular therapy settings and device deployment scenarios. Additional control icons relating to stimulation session control, e.g., Stop Stimulation icon 609, as well as any other icons relating to the remote care session such as lead/electrode selection 613, may be presented as minimized sub-panels adjacent to the control panel window 606 so as not to compromise the display area associated with the patient's image display 602.

In some embodiments, a code portion may be provided as part of the clinician programmer application to effectuate the transitioning of GUI screen 600 to or from a different sizing (e.g., resizing) in order to facilitate more expanded, icon-rich GUI screen in a different display mode. For example, a client device GUI screen may be configured such that the clinician's and patient's video images are presented in smaller windows, respectively, with most of the rest of the display region being populated by various icons, windows, pull-down menus, dialog boxes, etc., for presenting available programming options, lead selection options, therapy setting options, electrode selection options, and the like, in a more elaborate manner. In some embodiments, the video UI panels and related controls associated with clinician/patient video image windows may be moved around the GUI screen by “dragging” the images around the display area. Still further, the positioning of the video UI panels and related controls associated with clinician/patient video image windows may be stored as a user preference for a future UI setup or configuration that can be instantiated or initialized when the controller application is launched. As can be appreciated, it is contemplated that a clinician device may be configured to be able to toggle between multiple GUI display modes by pressing or otherwise activating zoom/collapse buttons that may be provided on respective screens.

In some further embodiments, a clinician device may be provided with additional functionality when utilizing or operating in the resized display GUI screen mode. By way of a suitable inputting mechanism at the clinician device, e.g., by pressing or double-tapping a particular portion of the patient's image, or by scrolling a cursor or a pointing device to a particular portion of the patient's image, etc., the clinician can remotely control the AV functionality of the patient controller device, e.g., a built-in camera or an auxiliary AV device such as AV equipment, in order to zoom in on and/or pan to specific portions of the patient's body in order to obtain close-up images that can enable better diagnostic assessment by the clinician. In such embodiments, zooming or enlarging of a portion of the patient's image, e.g., eye portion, may be effectuated by either actual zooming, i.e., physical/optical zooming of the camera hardware, or by way of digital zooming (i.e., by way of image processing).

In some embodiments, both optical and digital zooming of a patient's image may be employed. In still further embodiments, the patient controller device and/or associated AV equipment may be panned and/or tilted to different portions of the patient's body to observe various motor responses and/or conditions while different programming settings may be effectuated in a remote therapy session, e.g., shaking and tremors, slowed movement or bradykinesia, balance difficulties and eventual problems standing up, stiffness in limbs, shuffling when walking, dragging one or both feet when walking, having little or no facial expressions, drooling, muscle freezing, difficulty with tasks that are repetitive in nature (like tapping fingers or clapping hands or writing), difficulty in performing everyday activities like buttoning clothes, brushing teeth, styling hair, etc.

In still further embodiments, separate remote therapy session intervention controls (e.g., pause and resume controls) may be provided in addition to stimulation start and termination controls, which may be operative independent of or in conjunction with AV communication session controls, in a manner similar to example patient controller GUI embodiments set forth hereinbelow. Still further, data labeling buttons or controls may also be provided in a separate overlay or window of GUI screen 600 (not shown in FIG. 6) to allow or otherwise enable the clinician to provide different types of data labels for the AV data and therapy settings data that may be implemented in association with some example embodiments of the present patent disclosure.

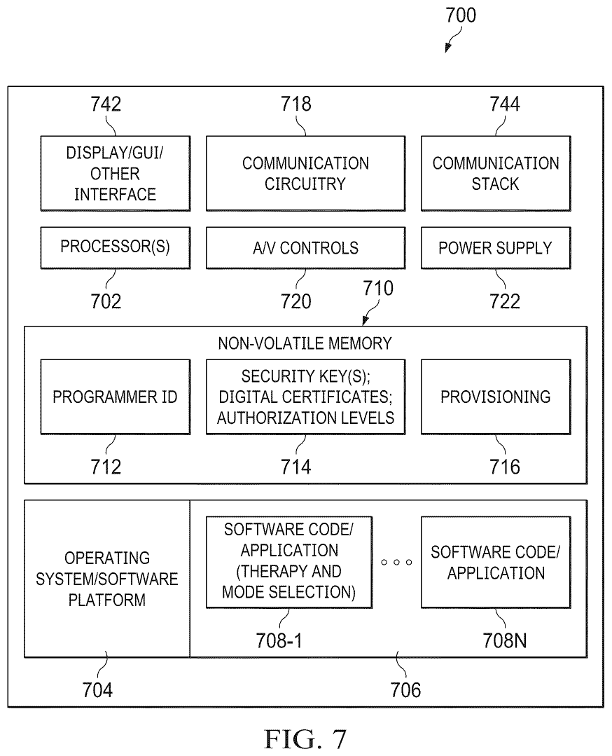

FIG. 7 depicts a block diagram of a generalized external edge device operative in a digital health network architecture for purposes of some embodiments of the present disclosure. For example, depending on configuration and/or modality, external device 700 may be representative of a clinician programmer device, a patient controller device, or a delegated device operated by an agent of a patient or a clinician, or a third-party device having subordinate levels of privileges/authorizations with respect to remote therapy and monitoring operations, e.g., limited to A/V session participation. Further, external device 700 may be a COTS device or non-COTS device as previously noted. Still further, external device 700 may be a device that is controlled and managed in a centralized enterprise device management system (EDMS), also referred to as a mobile/medical device management system (MDMS), which may be associated with the manufacturer of the IMDs and associated therapy application components in some embodiments (e.g., as an intranet implementation, an extranet implementation, or internet-based cloud implementation, etc.), in order to ensure that only appropriately managed/provisioned devices and users are allowed to engage in communications with IMDs with respect to monitoring the devices and/or providing therapy to patients using approved therapy applications, or allowed to join ongoing therapy sessions involving principal parties (e.g., the patients and clinicians). Still further, external device 700 may be a device that is not controlled and managed in such a device management system. Accordingly, it will be realized that external device 700 may comprise a device that may be configured in a variety of ways depending on how its functional modality is implemented in a particular deployment.

Example external device 700 may include one or more processors 702, communication circuitry 718 and one or more memory modules 710, operative in association with one or more OS platforms 704 and one or more software applications 708-1 to 708-K depending on configuration, cumulatively referred to as software environment 706, and any other hardware/software/firmware modules, all being powered by a power supply 722, e.g., battery. Example software environment 706 and/or memory 710 may include one or more persistent memory modules comprising program code or instructions for controlling overall operations of the device, inter alia. Example OS platforms may include embedded real-time OS systems, and may be selected from, without limitation, IOS, Android, Chrome OS, Blackberry OS, Fire OS, Ubuntu, Sailfish OS, Windows, Kai OS, eCos, LynxOS, QNX, RTLinux, Symbian OS, VxWorks, Windows CE, MontaVista Linux, and the like. In some embodiments, at least a portion of the software applications may include code or program instructions operative as one or more medical/digital health applications for effectuating or facilitating one or more therapy applications, remote monitoring/testing operations, data capture and logging operations, trial therapy applications, remote assistance, A/V session redirection to different endpoints, third-party enablement, etc. Such applications may be provided as a single integrated app having various modules that may be selected and executed via suitable drop-down menus in some embodiments. However, various aspects of the edge device digital healthcare functionalities may also be provided as individual apps that may be downloaded from one or more sources such as device manufactures, third-party developers, etc. By way of illustration, application 708-1 is exemplified as digital healthcare app configured to interoperate with program code stored in memory 710 to execute various operations relative to device registration, mode selection, remote/test/trial programming, therapy selection, security applications, and provisioning, A/V redirection, third-party enablement, privacy policy control, etc., as part of a device controller application.

In some embodiments of external device 700, memory modules 710 may include a non-volatile storage area or module configured to store relevant patient data, therapy settings, and the like. Memory modules 710 may further include a secure storage area 712 to store a device identifier (e.g., a serial number) of device 700 used during therapy sessions (e.g., local therapy programming or remote therapy programming). Also, memory modules 710 may include a secure storage area 714 for storing security credential information, e.g., one or more cryptographic keys or key pairs, signed digital certificates, etc. In some arrangements, such security credential information may be specifically operative in association with approved/provisioned software applications, e.g., therapy/test application 708-1, which may be obtained during provisioning. Also, a non-volatile storage area 716 may be provided for storing provisioning data, validation data, settings data, metadata etc. Communication circuitry 718 may include appropriate hardware, software and interfaces to facilitate wireless and/or wireline communications, e.g., inductive communications, wireless telemetry or M2M communications, etc. to effectuate IMD communications, as well as networked communications with cellular telephony networks, local area networks (LANs), wide area networks (WANs), packet-switched data networks, etc., based on a variety of access technologies and communication protocols, which may be controlled by the digital healthcare application 708-1 depending on implementation.

For example, application 708-1 may include code or program instructions configured to effectuate wireless telemetry and authentication with an IMD/NIMI device using a suitable M2M communication protocol stack which may be mediated via virtual/digital assistant technologies in some arrangements. By way of illustration, one or more bi-directional communication links with a device may be effectuated via a wireless personal area network (WPAN) using a standard wireless protocol such as Bluetooth Low Energy (BLE), Bluetooth, Wireless USB, Zigbee, Near-Field Communications (NFC), WiFi (e.g., IEEE 802.11 suite of protocols), Infrared Wireless, and the like. In some arrangements, bi-directional communication links may also be established using magnetic induction techniques rather than radio waves, e.g., via an induction wireless mechanism. Alternatively and/or additionally, communication links may be effectuated in accordance with certain healthcare-specific communications services including, Medical Implant Communication Service (MICS), Wireless Medical Telemetry Service (MTS), Medical Device Radiocommunications Service (MDRS), Medical Data Service (MDS), etc. Accordingly, regardless of which type(s) of communication technology being used, external device 700 may be provided with one or more communication protocol stacks 744 operative with hardware, software and firmware (e.g., forming suitable communication circuitry including transceiver circuitry and antenna circuitry where necessary, which may be collectively exemplified as communication circuitry 718 as previously noted) for effectuating appropriate short-range and long-range communication links for purposes of some example embodiments herein.

External device 700 may also include appropriate audio/video controls 720 as well as suitable display(s) (e.g., touch screen), video camera(s), still camera(s), microphone, and other user interfaces (e.g., GUIs) 742, which may be utilized for purposes of some example embodiments of the present disclosure, e.g., facilitating user input, initiating IMD/network communications, mode selection, therapy selection, etc., which may depend on the aspect(s) of a particular digital healthcare application being implemented.

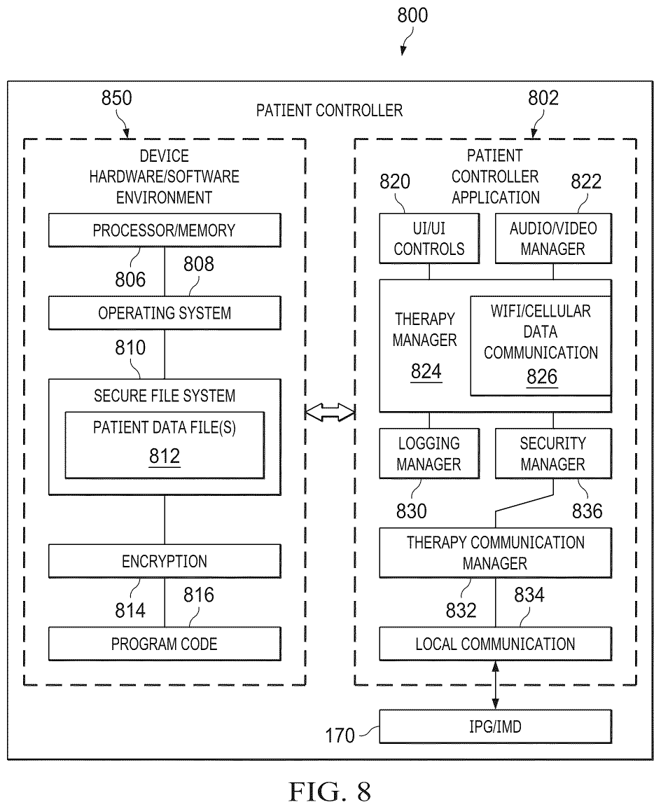

FIG. 8 depicts a block diagram illustrating additional details pertaining to a patient controller device operative in a digital health network architecture for purposes of some embodiments of the present disclosure. Example patient controller device 800 may be particularly configured for securely packaging and transmitting patient data to an external entity, e.g., a clinician programmer device and/or a network entity disposed in the digital health network in order to facilitate remote monitoring, AI/ML model training, and the like. Consistent with the description provided above with respect to a generalized edge device, patient controller device 800 may be provided with a patient controller application 802 configured to run in association with a suitable device hardware/software environment 850 effectuated by one or more processor and memory modules 806, one or more OS platforms 808, and one or more persistent memory modules 816 comprising program code or instructions for controlling overall operations of the device, inter alia. Example OS platforms may include a variety of embedded real-time OS systems as noted previously. In one implementation, a secure file system 810 that can only be accessed by the patient controller application 802 may be provided, wherein one or more patient data files 812 may be stored in a packaged encrypted form for secure transmission for purposes of some embodiments herein. Also, patient controller application 802 may include a therapy manager 824 operative to facilitate remote and/or non-remote therapy applications and related communications using one or more communication interfaces, e.g., interface 834 with an IPG/IMD 804 and network communications interface 836 with a network entity, as previously noted. A logging manager 830 associated with therapy manager 824 may be provided for logging data. A security manager 828 associated with therapy manager 824 may be provided for facilitating secure or trusted communications with a network entity in some embodiments. A therapy communication manager 832 may be provided for facilitating secure therapy communications between patient controller 800 and a clinician programmer (not shown in this FIG.). Therapy communication manager 832 may also be interfaced with local communication interface 834 to effectuate secure communications with the patient's IPG/IMD 804 using a suitable short-range communications technology or protocol as noted previously.

In still further arrangements, suitable software/firmware modules 820 may be provided as part of patient controller application 802 to effectuate appropriate user interfaces and controls, e.g., A/V GUIs, in association with an audio/video manager 822 for facilitating therapy/diagnostics control, file management, and/or other input/output (I/O) functions, as well as for managing A/V session redirection to auxiliary display devices, allowing third-party enablement, remote assistance, etc. Additionally, patient controller 800 may include an encryption module 814 operative independently and/or in association or otherwise integrated with patient controller application 802 for dynamically encrypting a patient data file, e.g., on a line-by-line basis during runtime, using any known or heretofore unknown symmetric and/or asymmetric cryptography schemes, such as the Advanced Encryption Standard (AES) scheme, the Rivest-Shamir-Adleman (RSA) scheme, Elliptic Curve Cryptography (ECC), etc.

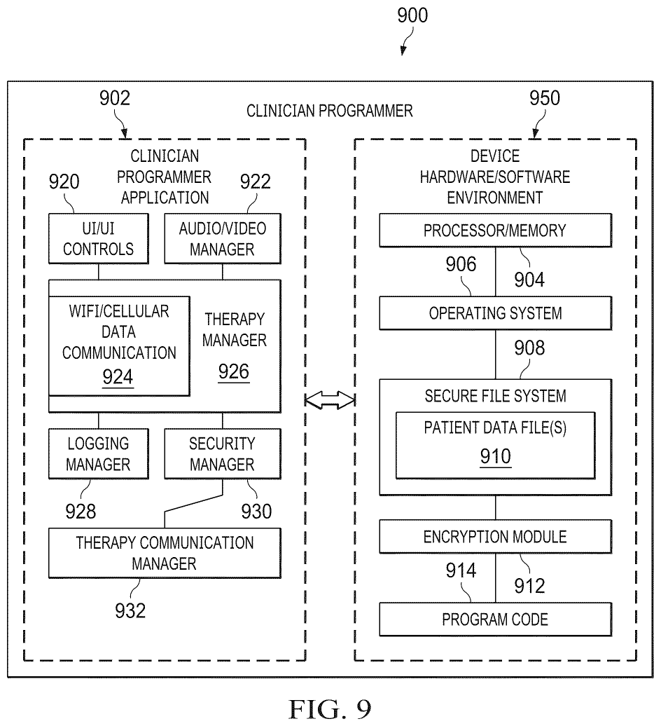

FIG. 9 depicts a block diagram illustrating additional details pertaining to a clinician programmer device operative in a digital health network architecture for purposes of some embodiments of the present disclosure. Similar to the example patient controller device 800 described above, example clinician programmer 900 may be particularly configured for facilitating secure transmission of patient data to an external entity, e.g., another clinician programmer device and/or a network entity disposed in the digital healthcare network in order to facilitate remote monitoring, AI/ML model training, and the like. A clinician programmer application 902 may be configured to run in association with a suitable device hardware/software environment 950 effectuated by one or more processor and memory modules 304, one or more OS platforms 906, and one or more persistent memory modules 914 comprising program code or instructions for controlling overall operations of the device, inter alia. As before, example OS platforms may include a variety of embedded real-time OS systems according to some embodiments. Further, a secure file system 908 may be provided in clinician programmer 900 that can only be accessed by the clinician programmer application 902, wherein one or more patient data files 310 (e.g., corresponding to one or more patients) may be stored in a packaged encrypted form, respectively, for purposes of some embodiments herein. In one implementation, clinician programmer application 902 may include a therapy manager 926 operative to facilitate remote and/or non-remote therapy applications and related communications using one or more communication interfaces, e.g., interface 924. For example, interface 924 may be configured to communicate with an IMD (not shown in this FIG.) using various short-range communication links with respect to in-person or in-clinic therapy according to some embodiments as previously noted. Likewise, example interface 924 may be configured to provide connectivity with wide-area networks for facilitating remote programming of an IMD and/or a telehealth session in some scenarios. In some arrangements, clinician programmer application 902 may also include functionality of facilitating suitable third parties to join an ongoing remote care or telehealth session with a patient as set forth in further detail elsewhere in the present disclosure. A logging manager 928 associated with therapy manager 924 may be provided for logging data for respective patients. A security manager 930 associated with therapy manager 926 may be provided for facilitating secure or trusted communications with a network entity in some embodiments. A therapy communication manager 932 may be provided for facilitating secure therapy communications between clinician programmer 900 and a patient controller (not shown in this FIG.). Suitable software/firmware modules 920 may be provided as part of clinician programmer application 902 to effectuate appropriate user interfaces and controls, e.g., A/V GUIs, in association with an audio/video manager 922 for facilitating therapy/diagnostics control, file management, and/or other I/O functions, as well as facilitating A/V session redirection, etc. as noted previously. Further, clinician programmer 900 may include an encryption module 912 similar to that of patient controller 800, wherein the encryption module 912 is operative in association and/or otherwise integrated with clinician programmer application 902 for encrypting a patient data file, e.g., dynamically on a line-by-line basis, during runtime using suitable techniques.

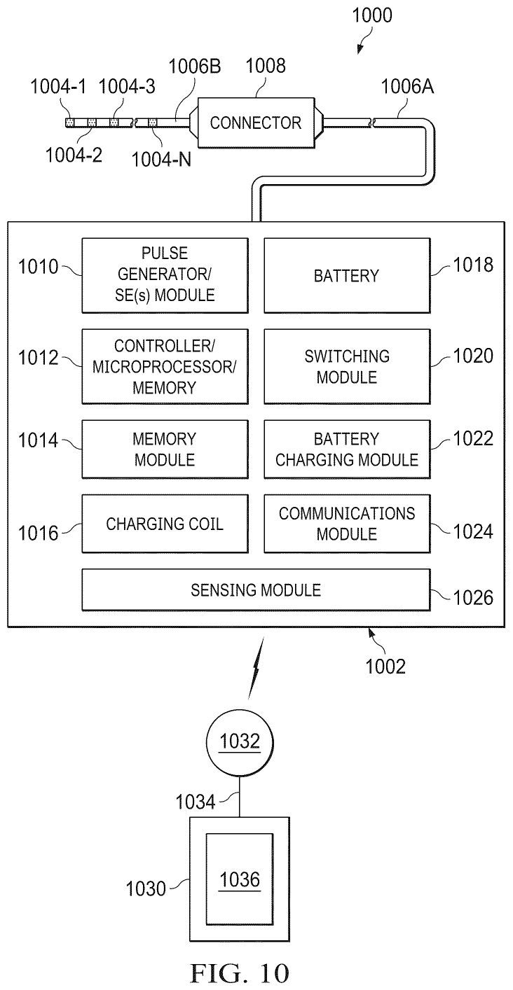

FIG. 10 depicts a block diagram of an IMD and associated system that may be configured for facilitating a remote care therapy application and/or a local therapy session for purposes of some example embodiments of the present disclosure. In general, therapy system 1000 may be adapted to generate electrical pulses to stimulate spinal cord tissue, peripheral nerve tissue, deep brain tissue, DRG tissue, cortical tissue, cardiac tissue, digestive tissue, pelvic floor tissue, or any other suitable biological tissue of interest within a patient's body, using an IMD or a trial IMD depending on implementation as previously noted. In one example embodiment, IMD 1002 may be implemented as having a metallic housing or can that encloses a controller/processing block or module 1012, pulse generating circuitry including or associated with one or more stimulation engines 1010, a charging coil 1016, a power supply or battery 1018, a far-field and/or near field communication block or module 1024, battery charging circuitry 1022, switching circuitry 1020, sensing circuitry 1026, a memory module 1014, and the like. IMD 1002 may include a diagnostic circuit module associated with a sensing module 1026 adapted to effectuate various diagnostics with respect to the state/condition of one or more stimulation electrodes and sensing electrodes of an implantable lead system as well as other bio/physiological sensors integrated or otherwise operative with IMD 1002. Controller/processor module 1012 typically includes a microcontroller or other suitable processor for controlling the various other components of IMD 1302. Software/firmware code, including digital healthcare application and encryption functionality, may be stored in memory 1014 of IMD 1002, and/or may be integrated with controller/processor module 1012. Other application-specific software code as well as associated storage components (not particularly shown in this FIG.) for execution by the microcontroller or processor 1012 and/or other programmable logic blocks may be provided to control the various components of the device for purposes of an embodiment of the present patent disclosure. As such, example IMD 1002 may be adapted to generate stimulation pulses according to known or heretofore known stimulation settings, programs, etc.

In one arrangement, IMD 1002 may be coupled (via a “header” as is known in the art, not shown in this FIG.) to a lead system having a lead connector 1008 for coupling a first component 1006A emanating from IMD 1002 with a second component 1006B that includes a plurality of electrodes 1004-1 to 1004-N, which may be positioned proximate to the patient tissue. Although a single lead system 1006A/1006B is exemplified, it should be appreciated that an example lead system may include more than one lead, each having a respective number of electrodes for providing therapy according to configurable settings. For example, a therapy program may include one or more lead/electrode selection settings, one or more sets of stimulation parameters corresponding to different lead/electrode combinations, respectively, such as pulse amplitude, stimulation level, pulse width, pulse frequency or inter-pulse period, pulse repetition parameter (e.g., number of times for a given pulse to be repeated for respective stimulation sets or “stimsets” during the execution of a program), etc. Additional therapy settings data may comprise electrode configuration data for delivery of electrical pulses (e.g., as cathodic nodes, anodic nodes, or configured as inactive nodes, etc.), stimulation pattern identification (e.g., tonic stimulation, burst stimulation, noise stimulation, biphasic stimulation, monophasic stimulation, and/or the like), etc. Still further, therapy programming data may be accompanied with respective metadata and/or any other relevant data or indicia.

As noted previously, external device 1030 may be deployed for use with IMD 1002 for therapy application, management and monitoring purposes, e.g., either as a patient controller device or a clinician programmer device. In general, electrical pulses are generated by the pulse generating circuitry 1010 under the control of processing block 1012, and are provided to the switching circuitry 1020 that is operative to selectively connect to electrical outputs of IMD 1002, wherein one or more stimulation electrodes 1004-1 to 1004-N per each lead 1006A/B may be energized according to a therapy protocol, e.g., by the patient or patient's agent (via a local session) and/or a clinician (via a local or remote session) using corresponding external device 1030. Also, external device 1030 may be implemented to charge/recharge the battery 1018 of IPG/IMD 1002 (although a separate recharging device could alternatively be employed), to access memory 1012/1014, and/or to program or reprogram IMD 1002 with respect to one or more stimulation set parameters including pulsing specifications while implanted within the patient. In alternative embodiments, however, separate programmer devices may be employed for charging and/or programming the IMD device 1002 device and/or any programmable components thereof. Software stored within a non-transitory memory of the external device 1030 may be executed by a processor to control the various operations of the external device 1030, including facilitating encryption of patient data logged in or by IMD 1002 and extracted therefrom. A connector or “wand” 1034 may be electrically coupled to the external device 430 through suitable electrical connectors (not specifically shown), which may be electrically connected to a telemetry component 1032 (e.g., inductor coil, RF transceiver, etc.) at the distal end of wand 1034 through respective communication links that allow bi-directional communication with IMD 1002. Alternatively, there may be no separate or additional external communication/telemetry components provided with external device 1030 in an example embodiment that uses BLE or the like for facilitating bi-directional communications with IMD 1002.

In a setting involving in-clinic or in-person operations, a user (e.g., a doctor, a medical technician, or the patient) may initiate communication with IMD 1002. External device 1030 preferably provides one or more user interfaces 1036 (e.g., touch screen, keyboard, mouse, buttons, scroll wheels or rollers, or the like), allowing the user to operate IMD 1002. External device 1030 may be controlled by the user through user interface 1036, allowing the user to interact with IMD 1002, whereby operations involving therapy application/programming, coordination of patient data security including encryption, trial IMD data report processing, third-party enablement, etc., may be effectuated.

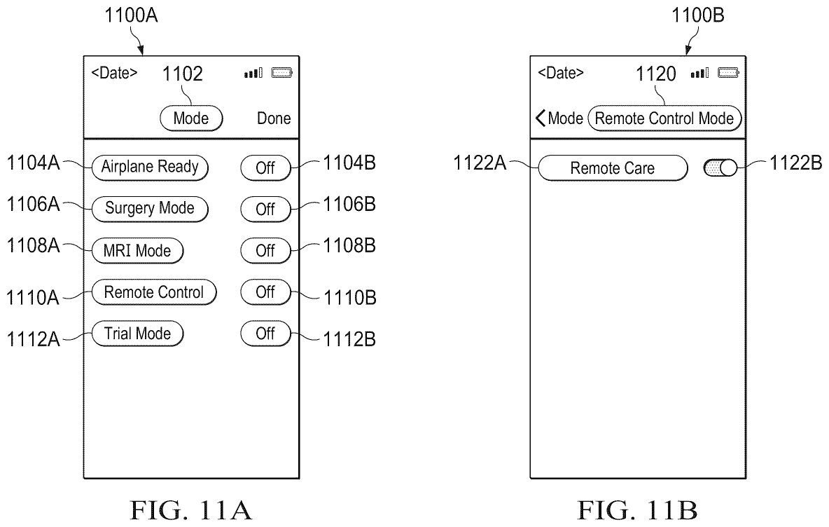

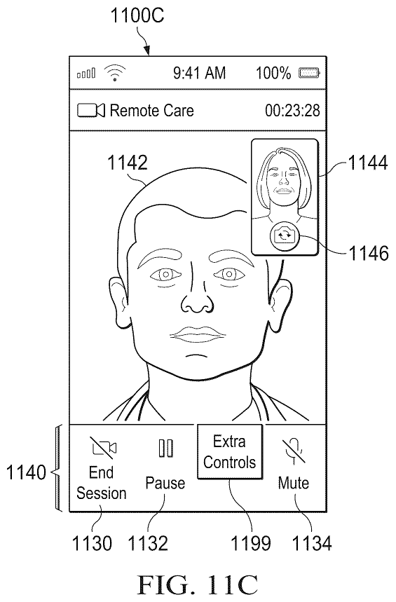

FIGS. 11A-11C depict representations of an example user interface and associated dialog boxes or windows provided with a patient controller device for selecting different therapy applications and/or service modes and for facilitating controls with respect to an AV communication session as well as a remote therapy session in an integrated remote care service application for purposes of some embodiments of the present disclosure. In some example implementations, a patient controller device, e.g. device 150 shown in FIG. 1A, may be provided with one or more non-transitory tangible computer-readable media or modules having program code stored thereon for execution on the patient controller device as part of or in association with a patient controller application, e.g., application 152, for facilitating remote therapy and telehealth applications in an integrated session having a common application interface. A code portion may be provided for displaying a mode selector icon on a GUI display screen of the patient controller device, wherein the mode selector icon is operative for accepting input by the patient to launch a remote care session with a clinician having a clinician programmer device. A code portion may be provided for displaying one or more audio controls and one or more video controls for facilitating an AV communication session or channel associated with the remote care session after the remote care session is established between the patient controller device and the clinician programmer device. Such AV controls may be represented as suitable icons, pictograms, and the like, e.g., a video/camera icon for controlling a video channel, a microphone icon for controlling an audio channel, a speaker icon for volume control, as well as control icons operative with respect to picture-in-picture (PIP) display regions, and the like. For example, video controls may be operative to effectuate a first display window and a second display window on the GUI display for respectively presenting an image of the clinician and an image of the patient in a PIP display mode. Yet another code portion may be provided for displaying one or more remote care therapy session controls in an overlay panel presented on the GUI display, wherein the one or more remote care therapy session controls are operative with respect to starting and ending a remote care therapy session by the patient as well as facilitating a temporary intervention or interruption of the therapy session while the AV communication session is maintained. As noted above, an example remote care therapy session may involve providing one or more programming instructions to the patient's IMD as part of the remote care session, and temporary intervention of the remote therapy may only suspend the remote programming of the patient's IMD although the AV communication session between the patient and the clinician remains active. In further embodiments, one or more code portions may be provided with the patient controller application to effectuate tactile controls with respect to different portions, fields, regions or X-Y coordinates of an active GUI display window that may be configured to interact with the functionality of the AV controls and/or therapy session controls. In still further embodiments, one or more code portions may be provided with the patient controller application to effectuate one or more data labeling buttons, icons, pictograms, etc., as part of the GUI display of the patient controller device, wherein the one or more data labeling buttons are operative to accept input by the patient corresponding to a subjective characterization of audio and/or video quality of the AV communications and/or other aspects of the therapy by the patient during the remote care session. In still further embodiments, one or more code portions may be provided with the patient controller application to facilitate patient input/feedback with respect to a (trial) therapy or treatment involving an IMD or a NIMI device, which may be augmented with one or more data labeling buttons, icons, pictograms, etc., wherein the patient input/feedback data may be provided to a network-based AI/ML model for facilitating intelligent decision-making with respect to whether the IMD/NIMI device should be deployed in a more permanent manner (e.g., implantation) and/or whether a particular therapy setting or a set of settings, including context-sensitive therapy program selection, may need to be optimized or otherwise reconfigured. In still further embodiments, one or more code portions may also be provided with the patient controller application to facilitate appropriate GUI controls, icons, dialog boxes, etc. for effectuating A/V session redirection, remote assistance, third-party and associated device enablement, etc. It should be appreciated that an example patent controller application may not necessarily include all such code portions in a particular implementation. Rather, an example patient controller application (and the device it is running on) may be configured for providing a smaller subset of functionalities and therefore fewer UI controls associated therewith.

As illustrated, FIGS. 11A and 11B depict example GUI screens 1100A and 1100B of a patient controller device that allow user input with respect to various mode settings/selections, including the activation and deactivation of allowing a remote control programming (i.e., therapy) session to be conducted, e.g., in trial therapy mode, etc. GUI display screen 1100A includes a mode selector 1102 that may be activated to show various mode settings which in turn may be selected, enabled or otherwise activated by using associated tactile controls. For example, modes such as “Airplane Ready” 1104A, “Surgery Mode” 1106A, “MRI Mode” 1108A, “Remote Control Mode” 1110A, “Trial Mode” 1112A, each having a corresponding swipe button 1104B-1112B are depicted. GUI screen 1100B illustrates a display that may be effectuated upon selecting or allowing Remote Control 1120 wherein a Remote Care Mode 1122A may be selected or enabled for activating remote therapy using a corresponding swipe button 1122B. A patient may therefore selectively permit the activation of remote therapy (i.e., remote programming of the IMD), whereby if activated and connected, a clinician can securely change or modify the therapy settings of the patient's IMD by effectuating appropriate therapy setting controls and associated GUIs provided at a controller device as previously set forth.

FIG. 11C depicts an example GUI display screen 1100C of the patient controller device during a remote care session, wherein an image of the selected clinician 1142 and an image of the patient 1144 may be presented in a PIP display region. In one display mode, the patient's image 1144 may be presented as a smaller offset or overlay image and the clinician's image 1142 may be presented as a main, larger image. In some embodiments, the patient image window 1144 may be moved around the UI screen by “dragging” the image around the viewing window allocated to the clinician image 1142. An image swap control 1146 may be provided to swap the PIP display regions in another display mode, whereby the patient's image 1144 may be presented as the main, larger image whereas the clinician's image 1142 may be presented in a smaller overlay window.

In some embodiments, a control panel 1140 may also be presented as part of the GUI screen 1100C, wherein various AV communication session controls and remote therapy session controls may be displayed as suitable icons, pictograms, etc., in a consolidated GUI display as noted above. A video session icon 1130 may be activated/enabled or deactivated/disabled to selectively turn on or off the video channel of the session. A microphone icon 1134 may be activated/enabled or deactivated/disabled to selectively turn on or off the audio channel of the session. A pause/resume icon 1132 may be activated/enabled or deactivated/disabled to selectively pause or suspend, or resume the remote therapy session involving remote programming of the patient's IMD or any other remote digital healthcare application executing on the patient controller. In some implementations, activating or deactivating the video session icon 1130 may also be configured to turn on or off the remote therapy session. In some implementations, separate remote therapy session controls (e.g., start control, end control, etc. in addition to pause and resume controls) may be provided that are operative independent of the AV communication session controls. Still further, additional icons/buttons 1199 may also be provided in a separate overlay or window of the GUI screen 1100C to allow or otherwise enable additional functionalities, e.g., A/V session redirection, remote assistance, enablement of third-party devices to join an ongoing session, privacy settings with respect to third parties allowed to join, etc., as noted previously. Although various UI controls and/or associated icons have been set forth in the foregoing description of an example patient controller GUI display, it should be appreciated that a particular implementation of a patient controller's GUI may depend on the specific controller application functionalities and capabilities as well as the deployment scenarios. Accordingly, a smaller subset of the UI controls/icons may be present in some example embodiments of a patient controller wherein one or more functionalities of a patient controller application executing thereon may be disabled or otherwise inactivated. Moreover, where third-party enablement functionalities are involved, some additional and/or alternative UI controls, menus, dialog boxes, etc., may be provided, as will be set forth in additional detail further below.

In a further embodiment of a digital health network architecture of the present patent disclosure, a digital health “app” may be installed on or downloaded to a patient controller device, e.g., patient controller device 1210 shown in FIG. 12, to permit a patient to report therapy outcomes for clinician review and analysis. An example of an existing digital health app that is available to patients in certain jurisdictions for reporting therapy outcomes to neurostimulation (including spinal cord stimulation) is the MYPATH™ (or myPath™) app from Abbott Laboratories (Plano, TX). The digital health app may use the network communications capabilities of patient controller device 1210 to communicate patient-reported data to patient report processing platform 1218. The clinician of a given patient may review the patient-reported data stored on platform 1218 to determine whether the therapy is working as expected and whether the patient requires reprogramming to optimize therapy. In some cases, the patient may provide the patient-reported data during a “trial” period which is used to evaluate the effectiveness of the therapy for the patient from a temporary external system before surgical implantation of the IMD. If the trial is successful, surgical implantation of the IMD may occur. Additionally or alternatively, the patient may provide the outcome data after surgical implantation of the IMD to allow monitoring of the patient's condition and response to the therapy to continue on an ongoing basis.

In some example arrangements, various pieces of data and information from the end points disposed in a digital healthcare network architecture, e.g., architecture 1260 shown in FIG. 12, may also be transmitted to one or more cloud-centric platforms without end user involvement, e.g., as background data collection processes, in addition to user-initiated secure data transfer operations. As previously noted, one or more remote data logging platforms 1216 of system 1200 (shown in FIG. 12) may be configured to obtain, receive or otherwise retrieve data from patient controller devices, clinician programmer devices and other authorized third-party devices. On an individual patient level and on a patient population basis, patient aggregate data 1250 may be available for processing, analysis, and review to optimize patient outcomes for individual patients, for a patient population as a whole, and for relevant patient sub-populations of patients.

Patient aggregate data (PAD) 1250 may include basic patient data including patient name, age, and demographic information, etc. PAD 1250 may also include information typically contained in a patient's medical file such as medical history, diagnosis, results from medical testing, medical images, etc. The data may be inputted directly into system 1200 by a clinician or medical professional. Alternatively, this data may be imported from digital health records of patients from one or more health care providers or institutions.

As previously discussed, a patient may employ a patient controller “app” on the patient's smartphone or other electronic device to control the operations of the patient's IMD or minimally invasive device. For example, for spinal cord stimulation or dorsal root stimulation, the patient may use the patient controller app to turn the therapy on and off, switch between therapy programs, and/or adjust stimulation amplitude, frequency, pulse width, and/or duty cycle, among other operations. The patient controller app may be adapted or otherwise configured to log such events (“Device Use/Events Data”) and communicate the events to system 1200 to maintain a therapy history for the patient for review by the patient's clinician(s) to evaluate and/or optimize the patient's therapy as appropriate.

PAD 1250 may include “Patient Self-Report Data” obtained using a digital health care app operating on patient controller devices 1210. The patient self-report data may include patient reported levels of pain, patient well-being scores, emotional states, activity levels, and/or any other relevant patient reported information. The data may be obtained using the MYPATH app from Abbott Labs as one example.

PAD 1250 may include sensor data. For example, IMDs of patients may include integrated sensors that sense or detect physiological activity or other patient states. Example sensor data from IMDs may include dated related to evoked compound action potentials (ECAPs), local field potentials, EEG activity, patient heart rate or other cardiac activity, patient respiratory activity, metabolic activity, blood glucose levels, and/or any other suitable physiological activity. The integrated sensors may include position sensing circuits and/or accelerometers to monitor physical activity of the patient. Data captured using such sensors can be communicated from the medical devices to patient controller devices and then stored within patient/clinician data logging and monitoring platform 1216. Patients may also possess wearable devices such as health monitoring products (heart rate monitors, fitness tracking devices, smartwatches, etc.). Any data available from wearable devices may be likewise communicated to monitoring platform 1216.

As previously discussed, patients may interact with clinicians using remote programming/virtual clinic capabilities of system 1200. The video data captured during virtual clinic and/or remote programming sessions may be archived by platform 1214. The video from these sessions may be subjected to automated video analysis (contemporaneously with the sessions or afterwards) to extract relevant patient metrics. PAD data 1250 may include video analytic data for individual patients, patient sub-populations, and the overall patient population for each supported therapy.

The data may comprise various data logs that capture patient-clinician interactions (“Remote Programming Event Data” in PAD 1250), e.g., individual patients' therapy/program settings data in virtual clinic and/or in-clinic settings, patients' interactions with remote learning resources, physiological/behavioral data, daily activity data, and the like. Clinicians may include clinician reported information such as patient evaluations, diagnoses, etc. in PAD 1250 via platform 1216 in some embodiments. Depending on implementation, the data may be transmitted to the network entities via push mechanisms, pull mechanisms, hybrid push/pull mechanisms, event-driven or trigger-based data transfer operations, and the like.

In some example arrangements, data obtained via remote monitoring, background process(es), baseline queries and/or user-initiated data transfer mechanisms may be (pre) processed or otherwise conditioned in order to generate appropriate datasets that may be used for training, validating and testing one or more AI/ML-based models or engines for purposes of some embodiments. In some example embodiments, patient input data may be securely transmitted to the cloud-centric digital healthcare infrastructure wherein appropriate AI/ML-based modeling techniques may be executed for evaluating the progress of the therapy trial, predicting efficacy outcomes, providing/recommending updated settings, etc.

In one implementation, “Big Data” analytics may be employed as part of a data analytics platform, e.g., platform 1220, of a cloud-centric digital health infrastructure 1212. In the context of an example implementation of the digital health infrastructure 1212, “Big Data” may be used as a term for a collection of datasets so large and complex that it becomes virtually impossible to process using conventional database management tools or traditional data processing applications. Challenges involving “Big Data” may include capture, curation, storage, search, sharing, transfer, analysis, and visualization, etc. Because “Big Data” available with respect to patients' health data, physiological/behavioral data, sensor data gathered from patients and respective ambient surroundings, daily activity data, therapy settings data, health data collected from clinicians, etc. can be on the order of several terabytes to petabytes to exabytes or more, it becomes exceedingly difficult to work with using most relational database management systems for optimizing, ranking and indexing search results in typical environments. Accordingly, example AI/ML processes may be implemented in a “massively parallel processing” (MPP) architecture with software running on tens, hundreds, or even thousands of servers. It should be understood that what is considered “Big Data” may vary depending on the capabilities of the datacenter organization or service provider managing the databases, and on the capabilities of the applications that are traditionally used to process and analyze the dataset(s) for optimizing ML model reliability. In one example implementation, databases may be implemented in an open-source software framework such as, e.g., Apache Hadoop, that is optimized for storage and large-scale processing of datasets on clusters of commodity hardware. In a Hadoop-based implementation, the software framework may comprise a common set of libraries and utilities needed by other modules, a distributed file system (DFS) that stores data on commodity machines configured to provide a high aggregate bandwidth across the cluster, a resource-management platform responsible for managing compute resources in the clusters and using them for scheduling of AI/ML model execution, and a MapReduce-based programming model for large scale data processing.

In one implementation, data analytics platform 1220 may be configured to effectuate various AI/ML-based models or decision engines for purposes of some example embodiments of the present patent disclosure that may involve techniques such as support vector machines (SVMs) or support vector networks (SVNs), pattern recognition, fuzzy logic, neural networks (e.g., ANNs/CNNs), recurrent learning, and the like, as well as unsupervised learning techniques involving untagged data. For example, an SVM/SVN may be provided as a supervised learning model with associated learning algorithms that analyze data and recognize patterns that may be used for multivariate classification, cluster analysis, regression analysis, and similar techniques for facilitating facial recognition, biometric identification, etc. with respect to some embodiments of the present disclosure. Given example training datasets (e.g., a training dataset developed from a preprocessed database or imported from some other previously developed databases), each marked as belonging to one or more categories, an SVM/SVN training methodology may be configured to build a model that assigns new examples into one category or another, making it a non-probabilistic binary linear classifier in a binary classification scheme. An SVM model may be considered as a representation of the examples as points in space, mapped so that the examples of the separate categories are divided by a clear gap that is as wide as possible (i.e., maximal separation). New examples are then mapped into that same space and predicted to belong to a category based on which side of the gap they fall on. In addition to performing linear classification, SVMs can also be configured to perform a non-linear classification using what may be referred to as the “kernel trick”, implicitly mapping their inputs into high-dimensional feature spaces. In a multiclass SVM, classification may typically be reduced (i.e., “decomposed”) to a plurality of multiple binary classification schemes. Typical approaches to decompose a single multiclass scheme may include, e.g., (i) one-versus-all classifications; (ii) one-versus-one pair-wise classifications; (iii) directed acyclic graphs; and (iv) error-correcting output codes.

In some arrangements, supervised learning may comprise a type of machine leaning that involves generating a predictive model or engine based on decision trees built from a training sample to go from observations about a plurality of features or attributes and separating the members of the training sample in an optimal manner according to one or more predefined indicators. Tree models where a target variable can take a discrete set of values are referred to as classification trees, with terminal nodes or leaves representing class labels and nodal branches representing conjunctions of features that lead to the class labels. Decision trees where the target variable can take on continuous values are referred to as regression trees. In some other arrangements, an embodiment of the present patent disclosure may advantageously employ supervised learning that involves ensemble techniques where more than one decision tree (typically, a large set of decision trees) are constructed. In one variation, a boosted tree technique may be employed by incrementally building an ensemble by training each tree instance to emphasize the training instances previously mis-modeled or mis-classified. In another variation, bootstrap aggregated (i.e., “bagged”) tree technique may be employed that builds multiple decision trees by repeatedly resampling training data with or without replacement of a randomly selected feature or attribute operating as a predictive classifier. Accordingly, some example embodiments of the present patent disclosure may involve a Gradient Boosted Tree (GBT) ensemble of a plurality of regression trees and/or a Random Forest (RF) ensemble of a plurality of classification trees, e.g., in pain score classification and modeling.

Depending on implementation, various types of data (pre) processing operations may be effectuated with respect to the myriad pieces of raw data collected for/from the subject populations, e.g., patients, clinicians, etc., including but not limited to sub-sampling, data coding/transformation, data conversion, scaling or normalization, data labeling, and the like, prior to forming one or more appropriate datasets, which may be provided as an input to a training module, a validation/testing module, or as an input to a trained decision engine for facilitating prediction outcomes. In some arrangements, example data signal (pre) processing methodologies may account for varying time resolutions of data (e.g., averaging a data signal over a predetermined timeframe, e.g., every 10 minutes, for all data variables), missing values in data signals, imbalances in data signals, etc., wherein techniques such as spline interpolation method, synthetic minority over-sampling technique (SMOTE), and the like may be implemented.

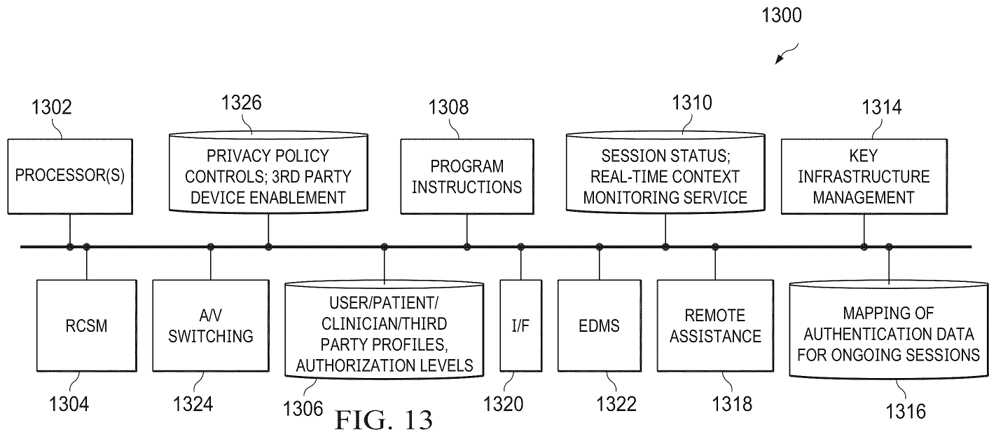

FIG. 13 depicts a block diagram of a system, apparatus or a computer-implemented platform that may be configured as a virtual clinic for purposes of some example embodiments of the present disclosure. It will be appreciated that apparatus 1300 may be implemented as part of a digital healthcare datacenter deployed in a network infrastructure as previously noted. In some arrangements, virtual clinic platform or apparatus 1300 may be configured in a loosely-coupled architecture, a tightly-coupled architecture, a distributed architecture, etc. with respect to the various components, modules and subsystems associated therewith. One or more processors 1302 may be operatively coupled to various modules that may be implemented in one or more persistent memory units for executing suitable program instructions or code portions (e.g., code portion 1308) with respect to effectuating remote device management, establishment of trusted associations, interfacing with encryption key management systems, and the like. In some arrangements, code portion 1308 may be executed in association with one or more other modules and/or databases of the platform 1300 for facilitating various functionalities such as remote care session management (RCSM), A/V session transfer, remote assistance, third-party enablement, privacy policy control, etc. As skilled artisans will recognize, the program instructions or code portions provided as part of the platform 1300 may therefore be configured in a number of ways in association with various modules and subsystems operative to execute one or more process flows described in the present disclosure. Accordingly, an RCSM module 1304, an A/V session redirection management module 1324, a real-time context monitoring service (RTCM) and session status module 1310, a key infrastructure management module 1314 and a device management module 1322 may be provided for implementing one or more example embodiments of the present disclosure. Databases and subsystems such as private policy control profiles 1326 that may be implemented in conjunction with third-party and associated device enablement, role-based temporal relationships, authentication data mapping for ongoing sessions 1316 as well as user/patient/clinician profiles and associated authorization levels 1306 may also be provided in an example implementation of VC platform 1300. Although not specifically shown herein, an IMD trust indicia verification module may be provided to create, maintain and store bonding associations between IMDs and clinician devices, which may be used for verification/bonding of patient controllers/devices in some embodiments. One or more patient history database(s) and user database(s) may also be provided in some embodiments wherein the database(s) are operative to maintain and store patients' historical data relating to different therapies, biophysiological conditions monitoring, etc., as well as patient profile data, clinician profile data, and the like, that may be provided as part of a database, e.g., database 1306. In some arrangements, therapy settings, stimulation application programs, stimulation level thresholds and limits, etc., may be stored in a related database, e.g., on a patient-by-patient basis and/or IMD-by-IMD basis, which may also include user-labeled therapy settings and session quality data, wherein the stored data may be made available to remote clinicians engaged in a particular therapy session. In some example arrangements, user-labeled data may be generated either at patient controllers, clinician programmers, or both, and appropriate user-labeled data records may be generated at either devices and/or at the platform 1300 under control of suitable program instructions configured as a record generator module. Example remote therapy applications supported by the platform 1300 may comprise, without limitation, an SCS therapy, a neuromuscular stimulation therapy, a dorsal root ganglion (DRG) stimulation therapy, a deep brain stimulation (DBS) therapy, a cochlear stimulation therapy, a drug delivery therapy, a cardiac pacemaker therapy, a cardioverter-defibrillator therapy, a cardiac rhythm management (CRM) therapy, an electrophysiology (EP) mapping and radio frequency (RF) ablation therapy, an electroconvulsive therapy (ECT), a repetitive transcranial magnetic stimulation (rTMS) therapy, a vagal nerve stimulation (VNS) therapy, and/or one or more physiological condition monitoring applications, among others, including any combinations or sub-combinations thereof. In some embodiments, a security management module may be provided as part of the platform 1300 for operating in association with key infrastructure management module 1314 for interfacing with and/or proxying clinician devices and patient devices with respect to facilitating secure remote therapy sessions including A/V and programming sessions for purposes of some example embodiments herein. In a further embodiment, example VC platform 1300 may include one or more AI/ML engines operative in association with one or more Big Data analytics module(s), not specifically shown herein. Further, in view of the flexible architecture of example VC platform 1300, a plurality of network interfaces (I/F) 1320 may be provided for interfacing with various external nodes or infrastructural elements, e.g., involving access and/core communications networks, external databases, cryptographic key infrastructure nodes, business support system nodes, third-party healthcare provider networks, and the like. In still further arrangements, various components, subsystems and modules of VC platform 1300 may be implemented as virtual elements configured to provide healthcare resources and services as part of an architecture involving Software as a Service (SaaS) services, Platform as a Service (PaaS) services, Infrastructure as a Service (IaaS) services, etc.

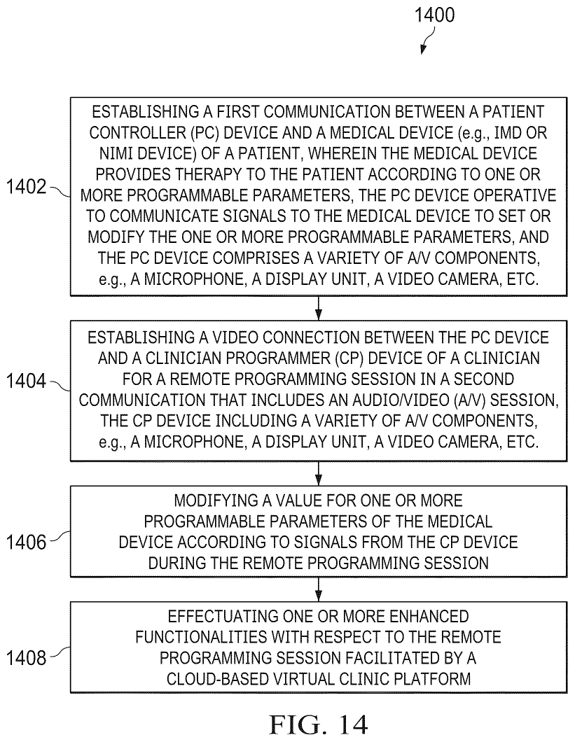

Turning to FIG. 14, depicted therein is a flowchart illustrative of blocks, steps and/or acts that may be (re) combined in one or more arrangements with or without additional flowcharts of the present disclosure for facilitating enhanced functionalities in a digital healthcare network deploying a VC platform according to some example embodiments. Example process flow 1400 may commence with establishing, at block 1402, a first communication between a patient controller (PC) device and a medical device (e.g., an IMD or NIMI device) of a patient, wherein the medical device may be configured to provide therapy to the patient according to one or more programmable parameters. In some example arrangements, the PC device may be operative to communicate signals to the medical device to set or modify the one or more programmable parameters, wherein the PC device may comprise, inter alia, a variety of A/V components, e.g. a microphone, a display unit, a video camera, etc. At block 1404, a video connection between the PC device and a clinician programmer (CP) device of a clinician may be established for facilitating a remote programming session in a second communication that may include an A/V session. The CP device may include, inter alia, a variety of A/V components, e.g., a microphone, a display unit, a video camera, etc. At block 1406, a value for one or more programmable parameters of the medical device may be modified according to signals from the CP device during the remote programming session. At block 1408, one or more enhanced functionalities (e.g., A/V redirection, third-party enablement, enforcement of privacy policies, etc.) may be effectuated with respect to the remote programming session facilitated by a cloud-based virtual clinic platform, e.g., VC platform 1300 set forth above.

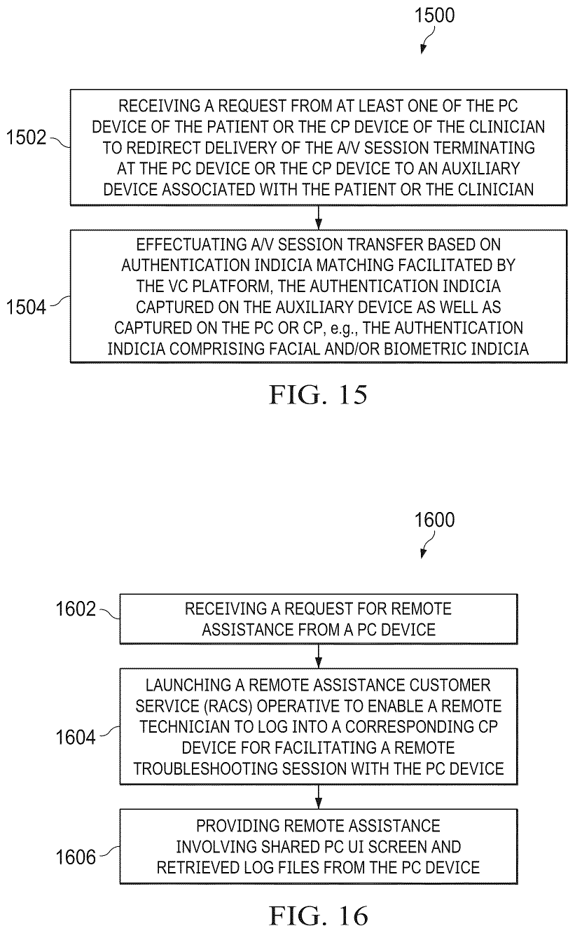

FIG. 15 depicts a flowchart illustrative of blocks, steps and/or acts that may be (re) combined in one or more arrangements with or without additional flowcharts of the present disclosure for facilitating securely switching/redirecting an A/V session between terminal endpoints during an integrated remote therapy session including programming in a digital healthcare network according to some example embodiments. As noted previously, a patient and a clinician may be engaged in a remote programming session using respective patient controller (PC) and clinician programmer (CP) devices facilitated by a VC platform disposed in a digital healthcare network. Example process flow 1500 may commence with receiving/generating a request from at least one of the PC device of the patient or the CP device of the clinician to redirect delivery of the A/V session terminating at the PC device or the CP device to an auxiliary device associated with the patient or the clinician (block 1502). In some example embodiments, the auxiliary device may comprise a device having a display operative to provide a higher quality video and/or support a larger viewing screen than that of either the PC device and/or the CP device. At block 1504, A/V session transfer or redirection may be effectuated responsive to comparing and matching one or more authentication indicia, which may be facilitated by the VC platform. In some arrangements, the authentication indicia may comprise indicia captured on the auxiliary device as well as captured on the PC or CP, e.g., the authentication indicia comprising facial and/or biometric indicia. Additional details with respect to the foregoing scheme will be set forth further below with respect to some example implementations of the present disclosure.

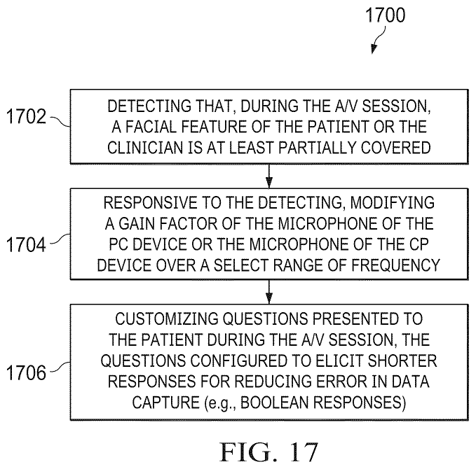

FIGS. 16 and 17 depict flowcharts illustrative of blocks, steps and/or acts that may be (re) combined in one or more arrangements with or without additional flowcharts of the present disclosure for facilitating remote analysis and assistance of an implantable device and/or associated patient controller deployed in a digital healthcare network according to some example embodiments. Example process flow 1600 may commence with receiving/generating a request for remote assistance from a PC device (block 1602). Responsive thereto, a remote assistance customer service (RACS) may be launched that may be operative to enable a remote technician to log into a corresponding CP device for facilitating a remote troubleshooting session with the PC device (block 1604). At block 1606, the remote technician may provide assistance based on shared PC UI screen as well as any retrieved log files from the PC device.

Example process flow of FIG. 17 may commence with detecting that, during the A/V session, a facial feature of the patient or the clinician (e.g., the mouth) is at least partially covered (block 1702). Responsive to the detecting, the functionalities and/or capabilities of certain hardware components associated with the PC or CP device may be suitably modified to compensate for any A/V quality degradation caused due to the obstructed facial feature. In some example embodiments, a gain factor of the microphone of the PC device or the microphone of the CP device may be modified/increased or otherwise optimized over a select range of frequency (block 1704). Depending on implementation, frequency ranges to be modified may range without limitation from, e.g., around 1 kHz to around 3 kHz. In some additional and/or alternative embodiments, any queries or questions presented to the patient during the A/V session may be customized, wherein the questions/queries are configured to elicit shorter responses from the patient, which may help reduce errors in audio capture/reception (e.g., Boolean responses such as “Yes” or “No”). Additional details with respect to schemes 1600, 1700 will be set forth further below with respect to some example implementations of the present disclosure.

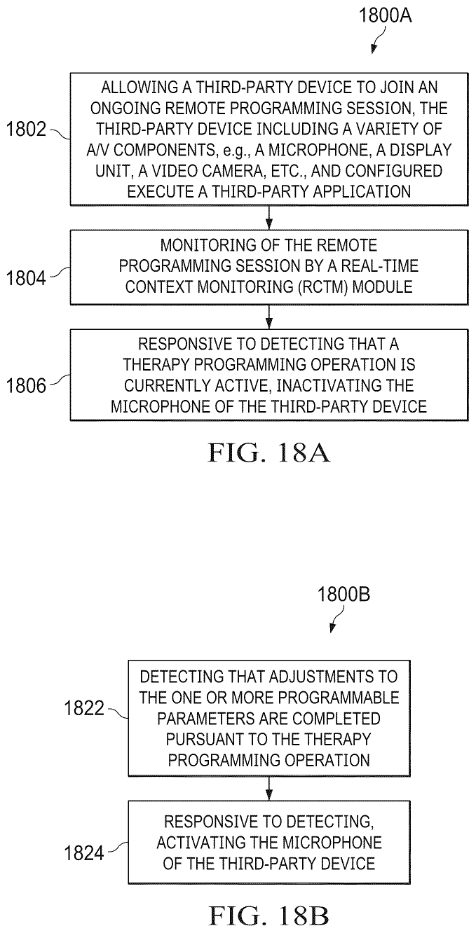

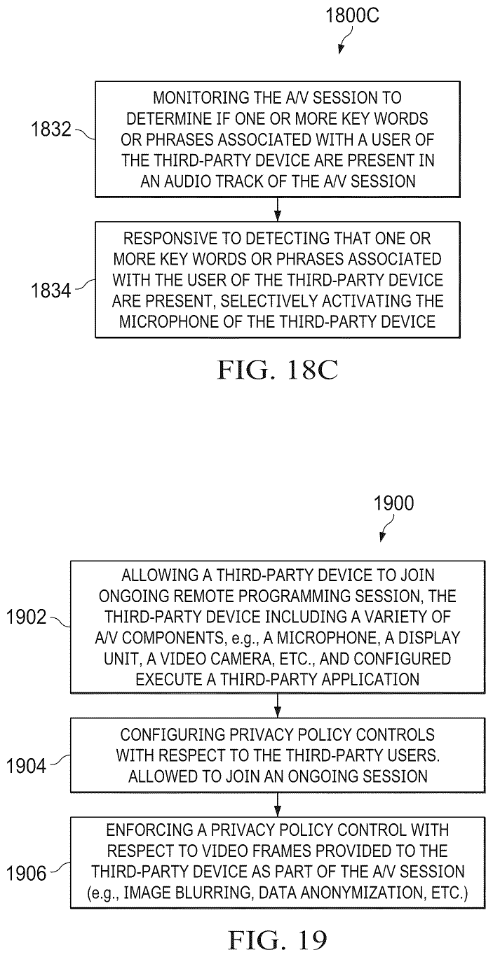

FIGS. 18A-18C depict flowcharts illustrative of blocks, steps and/or acts that may be (re) combined in one or more arrangements with or without additional flowcharts of the present disclosure for facilitating contextual notification and enablement of third-party/device participation in an ongoing remote therapy session including a remote programming session and associated A/V communication session in a digital healthcare network according to some example embodiments. Example process flow 1800A may commence with allowing a third-party and associated device to join an ongoing remote programming session, e.g., by a patient and/or a clinician, wherein the third-party device may comprise a variety of A/V components, e.g., a microphone, a display unit, a video camera, etc., and may be to configured execute a suitable healthcare application (referred to herein as a third-party application) that may be provisioned according to an approved device application provisioning service operating in conjunction with a suitable device management system (block 1802). The ongoing remote programming session may be monitored by a real-time context monitoring (RTCM) module (block 1804), which may be deployed locally and/or at a network node comprising a VC platform in some example embodiments. Responsive to detecting that a therapy programming operation is currently active (e.g., values of one or more programmable parameters of an IMD/NIMI device of the patient are being modified according to signals from the CP device that are proxied via the PC device), the functionalities and capabilities of certain hardware components of the third-party device may be deactivated or otherwise disabled (block 1808). In some arrangements, the microphone of the third-party device may be muted for the period during which the therapy programming is active. Additional aspects of facilitating a third party to join a session by way of suitable authorization will be set forth further below according to some examples of the present disclosure.

Example process flow 1800B of FIG. 18B is illustrative of a (re) activation of a third-party device according to some embodiments. At block 1822, a detection and/or determination is made that adjustments to the one or more programmable parameters of the IMD/NIMI device of the patient are completed pursuant to a current therapy programming operation. Responsive to the detecting/determining, the microphone of the third-party device may be (re) activated, i.e., unmuted, as set forth at block 1824. Example process flow 1800C of FIG. 18C is illustrative of a (re) activation of a third-party device according to still further embodiments. Process flow 1800C may commence with monitoring or continuing to monitor the A/V session of a remote therapy session to determine if one or more key words or phrases associated with a user of the third-party device are present in an audio track of the A/V session (block 1832). In some example embodiments, such “triggering” words or phrases may comprise whether the third-party device user's name is addressed, etc. Responsive to detecting/determining that one or more key words or phrases associated with the user of the third-party device are present in the audio track, the microphone of the third-party device may be selectively activated (block 1834).

FIG. 19 depicts a flowchart illustrative of blocks, steps and/or acts that may be (re) combined in one or more arrangements with or without additional flowcharts of the present disclosure for facilitating privacy policy control for multiple parties participating in a remote programming/therapy session effectuated by a virtual clinic deployed in a digital healthcare network according to some example embodiments. As before, example process flow 1900 may commence with allowing a third-party device to join an ongoing remote programming session, the third-party device including a variety of A/V components, e.g., a microphone, a display unit, a video camera, etc., and configured execute a third-party application (block 1902). In some example embodiments, various privacy policy controls with respect to the third-party users allowed to join the ongoing session may be selectively configured, e.g., by the patient, the clinician and/or a privacy policy management node associated with a VC platform (block 1904). Responsive to the applicable privacy policy configuration, a privacy policy control may be enforced with respect to video frames provided to the third-party device as part of the A/V session (block 1906). In some embodiments, techniques such as, e.g., image blurring, data anonymization, etc. may be applied to protect the identity and data privacy of the patient and/or the clinician. Additional details with respect to process flows 1800A-C and 1900 will be set forth further below with respect to some example implementations of the present disclosure.

In some implementations of a remote care therapy system, a virtual clinic may be configured to provide a telehealth and/or remote care session on select COTS devices having relatively small display screens and/or mediocre resolutions. For example, a smartphone may be configured to run a PC application whereas a tablet device may be configured to run a CP application. The size of the device's display can be an issue for some patients to view. Also, in some example embodiments where additional third parties are added to the session (e.g., a family member, a caregiver, second clinician, etc.), additional GUI controls, icons etc. can further clutter up the viewing screen. Accordingly, in some example embodiments of the present disclosure, PC devices and/or CP devices may be advantageously configured to have a control button that may be operative to start the process of switching from using the current terminals to a secondary/auxiliary device having better/larger display unit as described previously. In some implementations, a web-based application may be provisioned with the auxiliary device capable of joining a remote therapy session with just A/V. In some arrangements, a user (e.g., a patient or a clinician) may look into the camera of a first or primary device (e.g., operating as a PC device or a CP device) for capturing facial/biometric authentication data. The user may also look into the camera of a secondary/auxiliary device, which is operative to capture the user's facial/biometric authentication data. In some arrangements, the respective facial/biometric authentication data (e.g., images) may be uploaded to a backend authentication service associated with a VC platform that may be configured to confirm a match between the respective facial/biometric authentication data. Responsive to the confirmation, the A/V session may be redirected to the auxiliary device using a suitable network connection, including wireless, wireline, optical, terrestrial and satellite connections, using appropriate technologies. In some arrangements, upon the A/V handoff, the CP device may continue to be used for administering therapy and/or the PC device may continue to act as the proxy to patient's IMD/NIMI device.

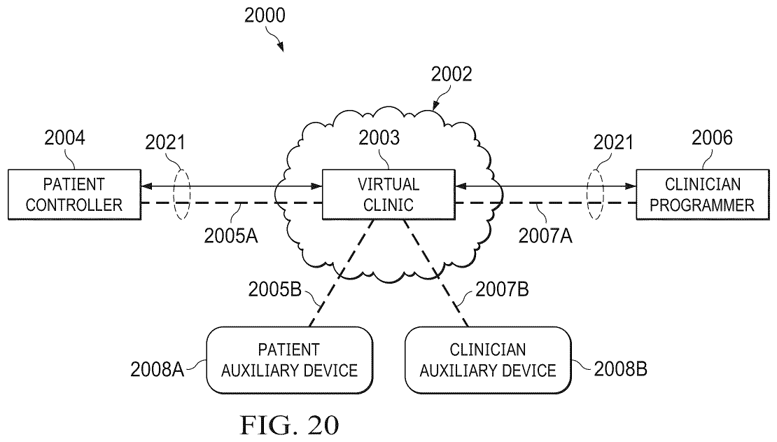

FIG. 20 depicts an example network architecture including a virtual clinic for facilitating A/V session redirection/switching during an integrated remote therapy session according to an implementation of the present disclosure. Example network architecture 2000 is a representative subset of the digital healthcare network environments exemplified in FIGS. 1A/1B and 12, wherein a PC device 2004 and a CP device 2006 are engaged in an ongoing remote care session 2021 facilitated by VC platform 2003 disposed in a network 2002. Responsive to a user input at PC 2004, VC platform 2003 redirects the patient's A/V path 2005A to the patient's auxiliary device 2008A as A/V path 2005B. Likewise, responsive to a user input at CP 2006, VC platform 2003 redirects the clinician's A/V path 2007A to the clinician's auxiliary device 2008B as A/V path 2007B. In some arrangements, both patient's A/V path 2005A and clinician's A/V path 2007A may be redirected to respective auxiliary devices 2008A, 2008B.

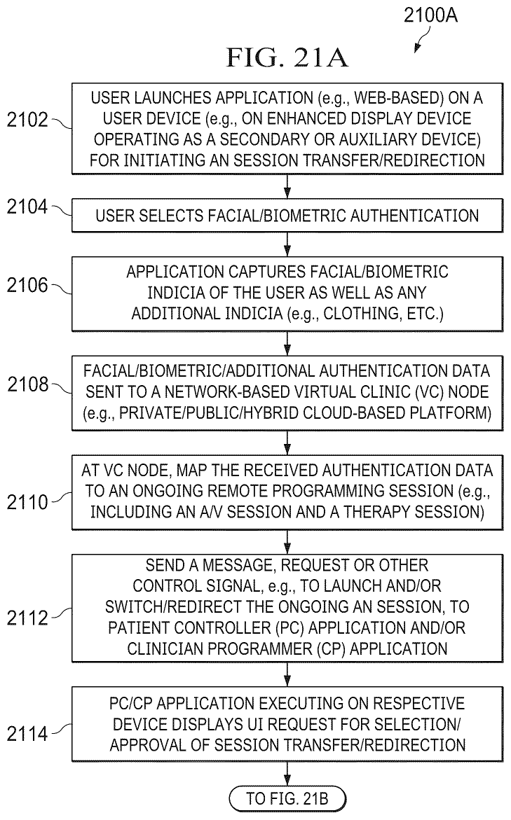

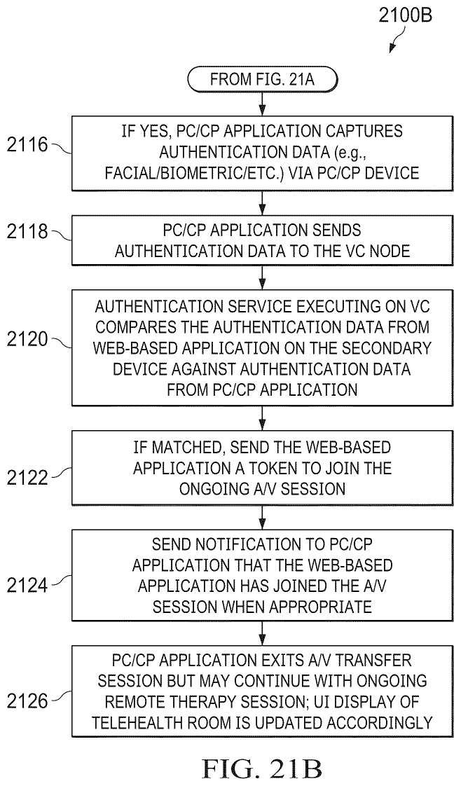

FIGS. 21A and 21B depict a flowchart illustrative of blocks, steps and/or acts of a process with additional details for effectuating redirection/switching of an A/V session to an auxiliary/secondary device associated with a patient and/or a clinician according to an implementation of the present disclosure. Process flow portions 2100A and 2100B shown in FIGS. 21A and 21B, respectively, comprise an example process 2000, which may commence with a user launching an application (e.g., web-based) on a user device, e.g., on enhanced display device, operating as a secondary or auxiliary device, for initiating A/V session transfer/redirection (block 2102). At block 2104, the user (e.g., the patient and/or the clinician) selects a facial/biometric authentication option presented via a display window facilitated by the web-based application, which may be configured to capture the facial/biometric indicia of the user as well as any additional indicia (e.g., clothing, etc.), as set forth at block 2106. The user may also be presented with an option to select/request access to connect to an existing remote programming session in which the user is currently engaged. Responsive to the user's request/selection, the facial/biometric/additional authentication data of the user may be sent to a network-based virtual clinic (VC) node (e.g., deployed in private/public/hybrid cloud-based platform), as set forth at block 2108. In some arrangements, the VC node may be configured with a mapping functionality for mapping or otherwise associating the received authentication data to an ongoing remote programming session (e.g., including an A/V session and a therapy session) (block 2110). Where the user has not initially selected a redirection option on the user's primary device (e.g., the PC or the CP device) and uploaded appropriate facial/biometric authentication data, a control message may be transmitted to the primary device to select/approve A/V path redirection, as set forth at blocks 2112, 2114, which may be selectively/optionally implemented in some arrangements. Upon approval, the PC/CP application may capture the user's facial/biometric authentication data, which may be uploaded to the VC platform (blocks 2116, 2118). The authentication mapping service executing at the VC platform compares the authentication data from the web-based application on the secondary device against the authentication data from the PC/CP application (block 2120). Responsive to determining that the respective facial/biometric authentication data match, a telehealth/remote care room token is sent to the web application to join the ongoing A/V session (block 2122). The VC platform may also notify the PC or CP application that the web application has joined in the ongoing A/V session (block 2124). In some arrangements, the PC or CP application may inquire the user if they joined in the A/V session on the respective auxiliary device. If so, the UI of the respective PC and CP applications may exit from the telehealth room while still indicating that the therapy session is ongoing (block 2126). Skilled artisans will recognize upon hereto that the foregoing scheme may be modified in various ways depending on, e.g., the sequential order of where and when the decision(s) to redirect an A/V session is (are) made.

In some implementations of a digital healthcare system operative to provide remote care therapy, various bots (short for “robots”, which may comprise a software program configured to perform a number of automated, repetitive, predefined tasks) may be employed to interact with patients and/or clinicians using voice, text, chat, etc. While interacting with digital services, it is important that voice input from the human actors, e.g., the patients, clinicians, and any third-party agents authorized to join ongoing sessions, remains clear enough that it is interpreted properly and acted upon accordingly by the applicable entities. Where the human actors are wearing masks, veils, or other articles of clothing that cover the actors' mouths, it become challenging to make out clearly what is being spoken as voice input. Example embodiments herein advantageously provide a scheme to learn the context of the user's facial features and adjust the capabilities of the A/V hardware of the user equipment. As set forth above with respect to the process flow 1700 of FIG. 17, some arrangements may be configured with the capability of contextual detection as to whether the user's mouth is covered (e.g., by hand, mask, etc.) and correspondingly adjusting the microphone gain over a range of frequencies. Additionally and/or alternatively, appropriate signal processing may be effectuated to filter low frequencies (e.g., around 400 Hz) to further improve performance and/or quality parameters such as signal-to-noise ratio (SNR) and signal-to-interference and noise ratio (SINR), etc. of the A/V circuitry of the device.

In some implementations of a digital healthcare system, where medical devices, PC/CP devices, third-party devices, etc., are migrating to advanced communication network architectures (e.g., 5G) including All-IP interoperability, which may include automated digital interactive systems, various challenges may be encountered by human actors, e.g., patients, etc. in setting up, using and troubleshooting the new technologies embodied in a device. Consequently, the patients may often require the help of medical representatives and/or call center personnel to help them debug, train, and fix any technical issues encountered with respect to their device. In order to facilitate remote assistance with respect to the issues encountered by the patients, an example VC platform may be provided with the functionality to provision appropriate technical personnel and/or customer service representatives wherein a secure telehealth assistance session may be effectuated that allows a remote technician to remotely analyze a PC application for current and historical errors. Accordingly, such authorized personnel and associated devices may be provisioned with appropriate roles and/or profiles for logging in a remote assistance application executing on respective devices, which may be maintained in a database associated with a VC platform implementation, e.g., VC platform 1300. In some embodiments, the telehealth assistance service may be configured to enable sharing of the patient's device screen with the remote technician to help monitor, examine, debug and fix any technical issues. In one implementation, the patient and a service representative or call center technician may establish a secure remote session where the patient controller application is mirrored, thereby providing the service representative and/or call center technician the ability to interact with the patient and adjust setting/parameters as if they were the patient. In other words, the service representative and/or call center technician may be provided suitable access (albeit under supervision in some arrangements) to go through the device, application software components, A/V hardware components, I/O components, OS components, etc., consistent with the remote technician's authorization profile setup.

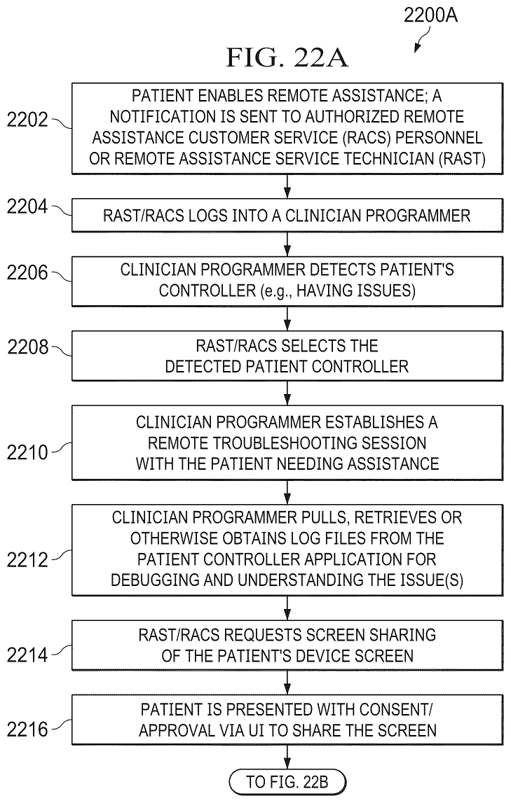

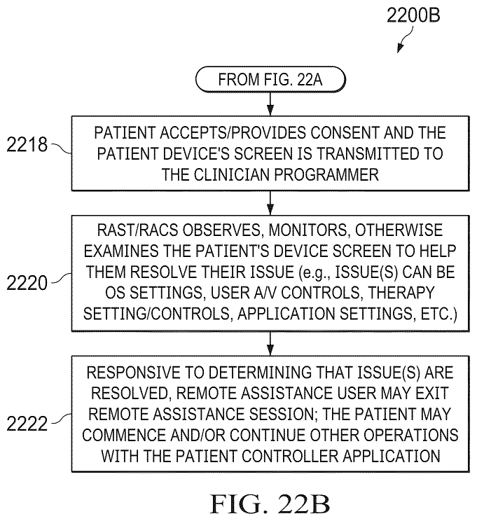

FIGS. 22A and 22B depict a flowchart illustrative of blocks, steps and/or acts of a process with additional details for effectuating a remote assistance procedure with respect to a patient controller device according to an implementation of the present disclosure. Process flow portions 2200A and 2200B shown in FIGS. 22A and 22B, respectively, comprise an example process 2200, which may commence with a patient enabling remote assistance by activating a suitable control icon on the PC device display. A notification may be sent to an authorized remote assistance customer service (RACS) personnel and/or remote assistance service technician (RAST), as set forth at block 2202. Responsive thereto, RAST/RACS personnel may log into a clinician programmer device (block 2204). In some arrangements, the CP device that RAST/RACS personnel is authorized to log into may be associated with the requesting patient's PC device. At block 2206, the CP application detects the patient's controller (e.g., identified with the patient having technical issues). At block 2208, RAST/RACS personnel may select the detected patient controller. At block 2210, the CP application establishes a remote troubleshooting session with the patient needing assistance. At block 2212, the CP application pulls, retrieves or otherwise obtains log files from the patient controller application for debugging and understanding the issue(s). At block 2214, RAST/RACS personnel requests sharing of the patient's device screen. At block 2216, the patient is presented with a request for consent/approval to share the screen. At block 2218, the patient accepts the request and provides consent. Responsive thereto, the patient device's screen is transmitted to the clinician programmer. At block 2220, RAST/RACS personnel may observe, monitor, and/or otherwise examine the patient's device screen to help resolve any issues (e.g., issues with OS settings, user A/V controls, therapy settings/controls, application settings, etc.). Responsive to determining that issue(s) are resolved, remote assistance user (e.g., RAST/RACS personnel) may exit remote assistance session. The patient may commence and/or continue other operations on the PC device in conjunction with the patient controller application. These operations are set forth at block 2222 of process flow portion 2200B.

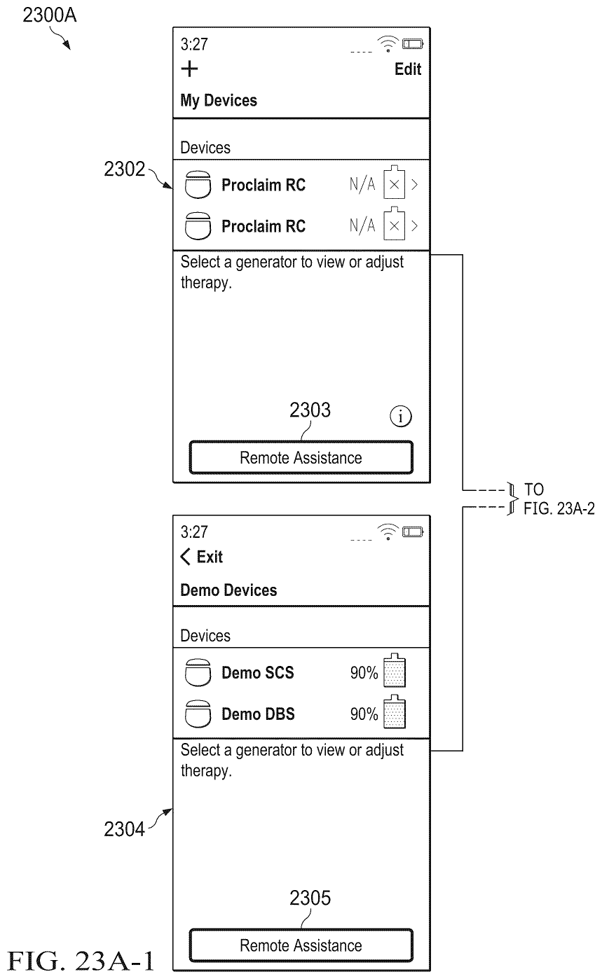

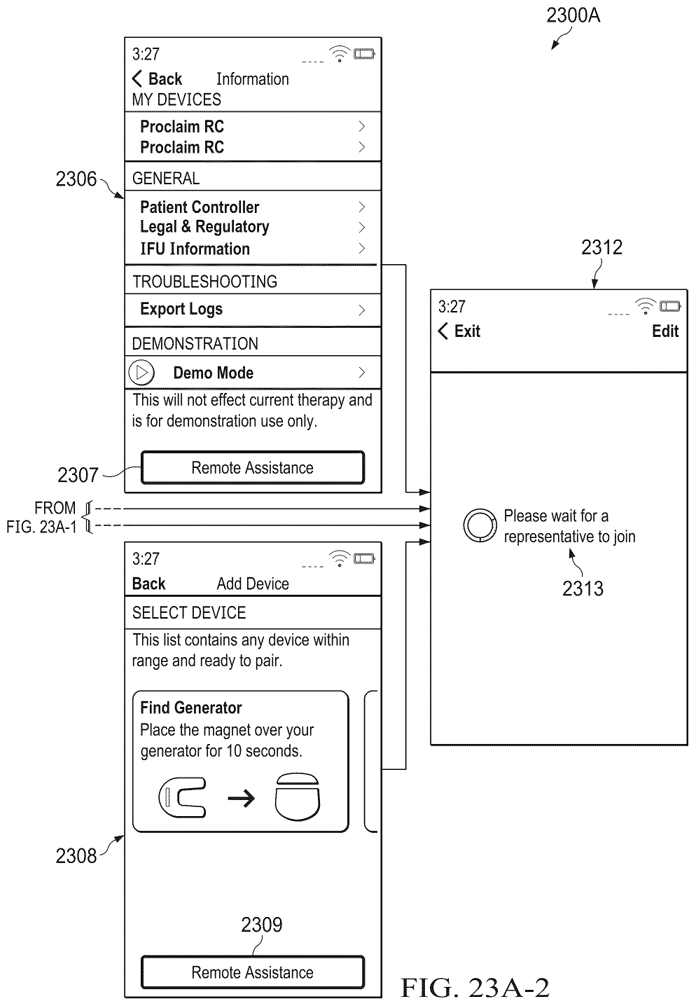

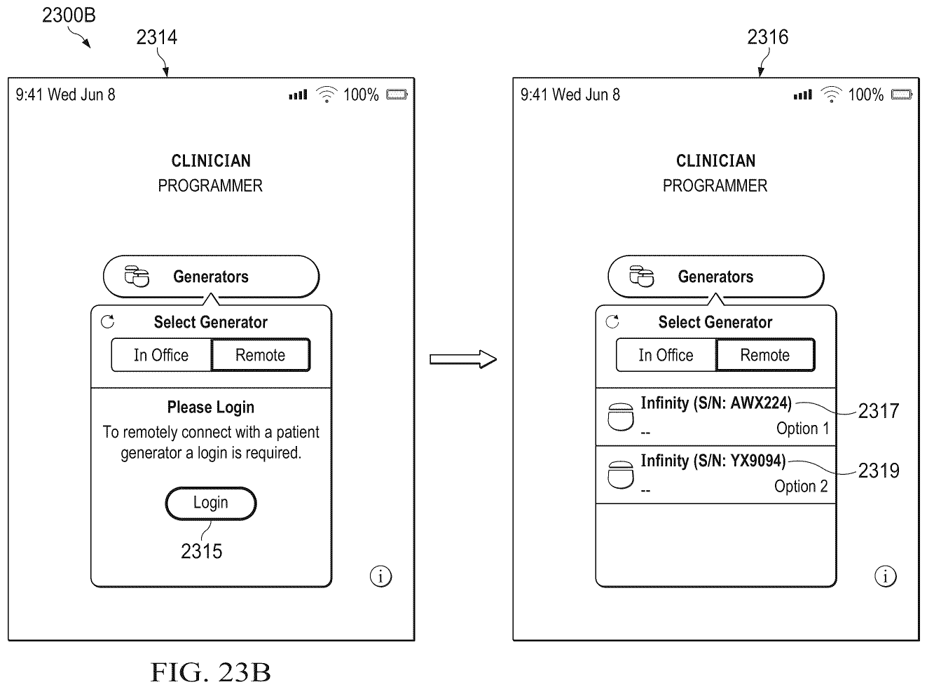

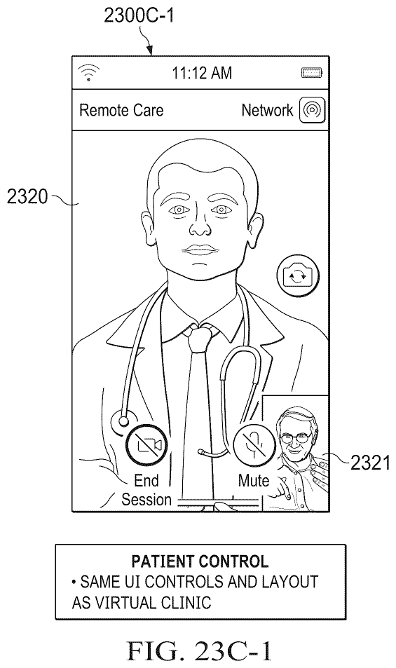

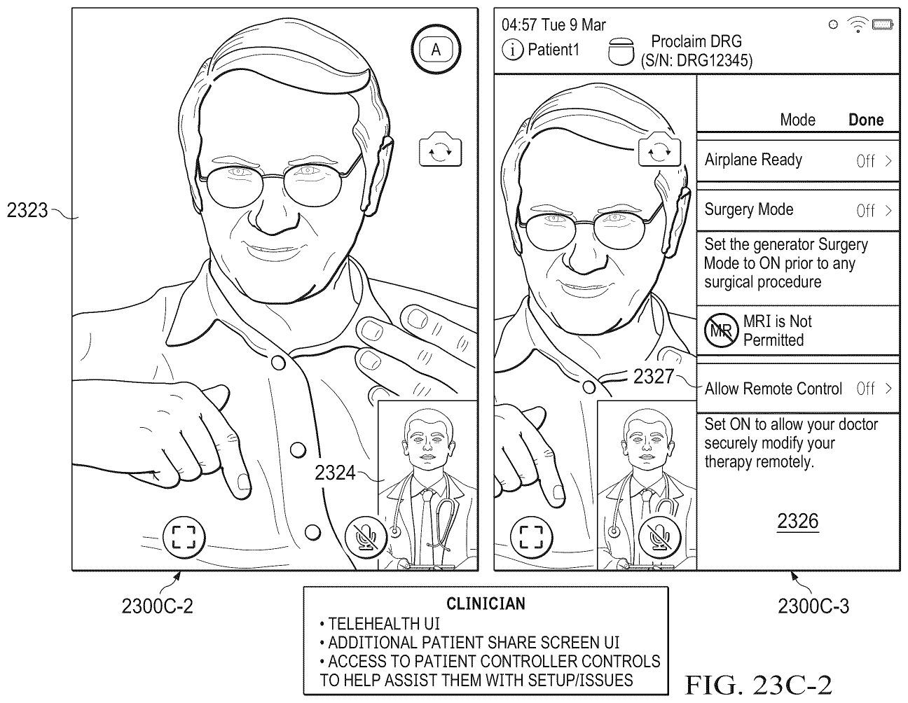

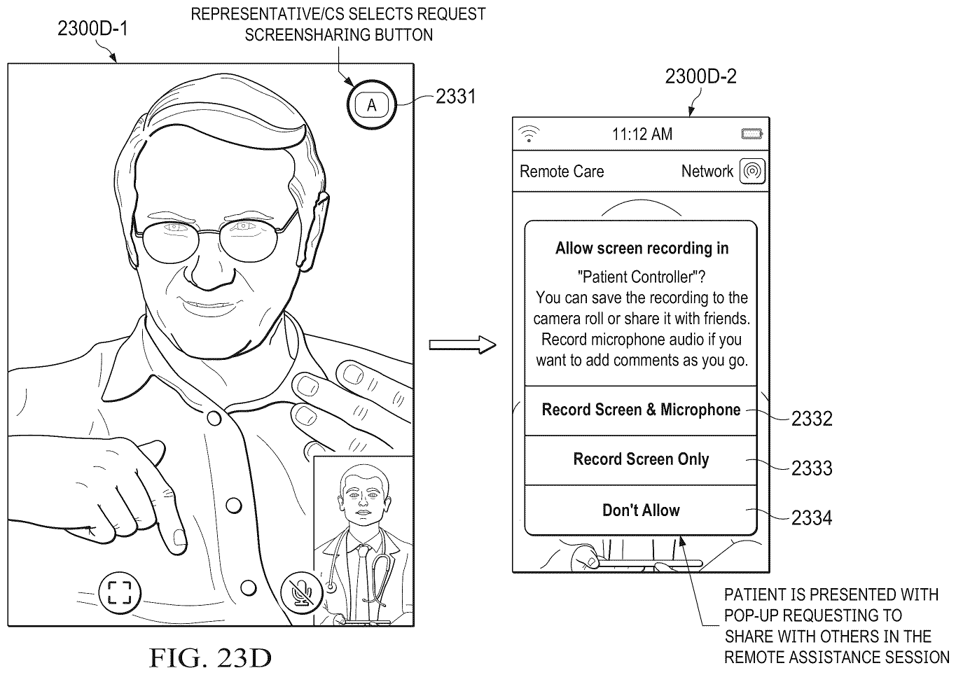

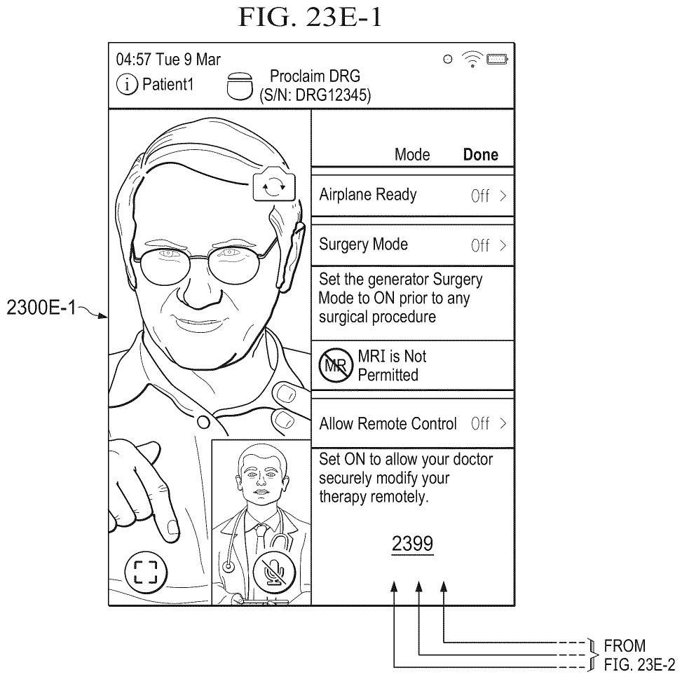

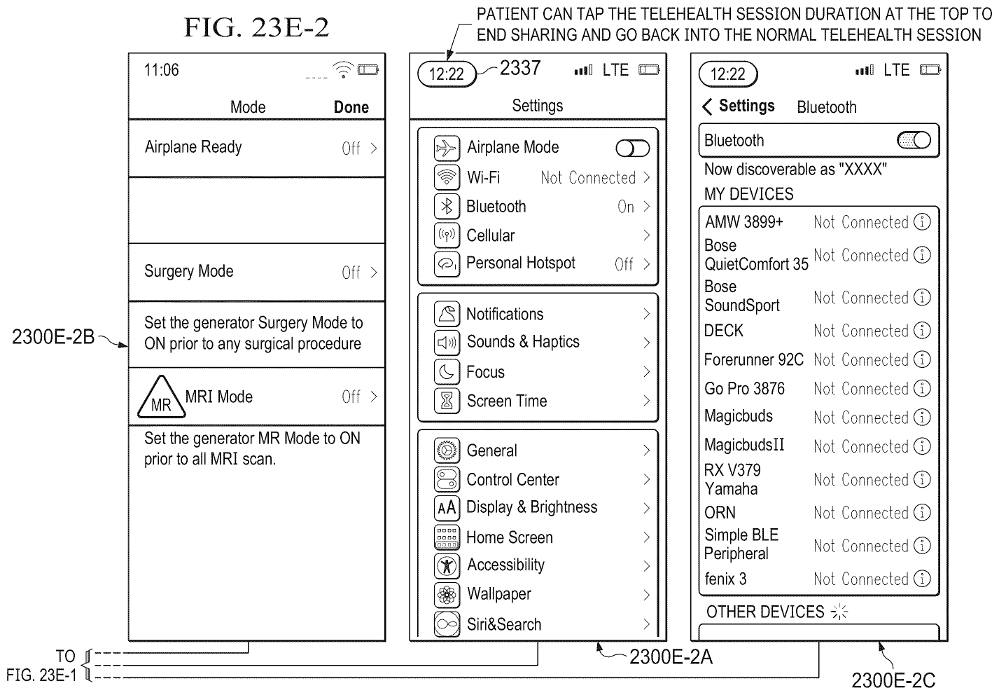

FIGS. 23A-1 and 23A-2 together depict various screenshot views relating to effectuating a remote assistance procedure according to some implementations of the present disclosure. Likewise, FIG. 23B, FIGS. 23C-1 and 23C-2, FIG. 23D, and FIGS. 23E-1 and 23E-2 depict one or several screenshot views relating to effectuating a remote assistance procedure according to some implementations of the present disclosure, wherein some of the processes may be (re) combined and/or (re) arranged.

Example screenshot views collectively shown at reference numeral 2300A in FIGS. 23A-1 and 23A-2 represent GUI screen presentations 2302, 2304, 2306, 2308 of a PC device in different modes or functionalities, wherein a patient controller application is operative to provide a Remote Assistance button that places the device into a telehealth session with approved RAST/RACS personnel. By way of illustration, the PC device may be in different modes such as, e.g., demo mode, add devices mode, display of current IMDs mode, etc. In some arrangements, GUI screen presentations 2302, 2304, 2306, 2308 may each display a respective Remote Assistance button 2303, 2305, 2307, 2309, regardless of the mode/functional stage of the device. Responsive to activating the button 2303, 2305, 2307, 2309, the patient may be “placed” or “ushered” into a telehealth virtual waiting room, e.g., as illustrated in UI view 2312. In some example implementations, a notification 2313 (e.g., “Please wait for a Representative to join”) may be provided to the patient to continue to wait until an approved RAST/RACS person joins.

Example screenshot views collectively shown at reference numeral 2300B in FIG. 23B represent GUI screen presentations 2314, 2316 with respect to allowing RAST/RACS personnel to receive patient requests/notifications regarding remote assistance. GUI presentation 2314 is illustrative of a display view presented at a client programmer device, wherein a login button 2315 may be provided to allow RAST/RACS personnel to log into the CP application to receive remote assistance notifications. After logging into the CP device, RAST/RACS personnel may select the patient's device from a list of devices 2317, 2319. Responsive to selecting a particular patient's device, a secure telehealth session may be established with the PC device.

Example PC device GUI screen 2300C-1 shown in FIG. 23C-1 is representative of a display view after establishing a telehealth session pursuant to a remote assistance request. In some example embodiments, GUI 2300C-1 may be substantially similar to the display layout and UI controls/buttons provided on the PC device while in a virtual clinic session. In some arrangements, a larger clinician image display portion 2320 and a smaller patient image display portion 2321 may be provided as part of GUI screen 2300C-1, similar to some embodiments described hereinabove with respect to FIGS. 11A-11C. Example CP device GUI screen presentations may include a first display mode screen 2300C-2 (shown in FIG. 23C-2), wherein an entire display view may be partitioned between a larger patient image display portion 2323 and a smaller clinician image display portion 2324. A second display mode screen 2300C-3 associated with the CP device may include a split screen wherein a screen portion 2326 may include an additional UI view for displaying a shared patient controller screen upon obtaining consent/approval from the patient via a suitable button 2327 that allows remote control, as shown in FIG. 23C-2.

Example screenshots 2300D-1 and 2300D-2 shown in FIG. 23D represent GUI screen presentations at the CP device and PC device, respectively, that illustrate screen-sharing request and permission generation. As illustrated in GUI screen presentation 2300D-1, RAST/RACS personnel may select a Request Screen Sharing button 2331. Responsive thereto, the patient may be presented with a display window requesting to allow screen-sharing with others in the remote assistance session. As illustrated in GUI screen presentation 2300D-2, various options may be provided to the patient depending on implementation, such as, e.g., relating to recording of the screen-sharing and voice option 2332, recording screen-sharing only option 2333, or not allowing screen-sharing option 2334.

Example screenshots 2300E-1 and 2300E-2A to 2300E-2C shown in FIGS. 23E-1 and 23E-2, respectively, represent GUI screen presentations at the CP device and PC device, respectively, that illustrate screen-sharing during a remote session. As illustrated in GUI screen 2300E-1 associated with the CP device, a split screen portion 2399 may be provided for displaying the screen of the PC device that may be put in different modes or stages. GUI screen presentations 2300E-2A to 2300E-2C are representative of a plurality of display screen views exemplified for sharing with the CP device under a remote assistance session. In some example embodiments, the patient controller application may be operative to provide a Stop button 2337, e.g., at a top corner of the display screen, to end sharing and return to the normal telehealth session.

In some implementations of a remote care therapy system, a virtual clinic may be configured to provide a telehealth and/or remote care session involving only principal parties, e.g., the patient and the clinician. In some example arrangements, additional parties may be authorized as third parties to join an ongoing session, e.g., family members, caregivers, additional clinician or medical personnel, etc. as noted previously. In such arrangements, it is important that the third parties do not interrupt, distract and/or otherwise impede a therapy operation where the therapy parameters are adjusted, or when the patient and the client are engaged in the middle of examination. Some example embodiments may therefore be advantageously configured to facilitate the addition of one or more third parties operating suitably provisioned user equipment, wherein the third-party application executing thereon is adapted to provide an A/V interface that allows the third parties to see and hear the patient and the clinician while being contextually monitored. In some arrangements, accordingly, the ability for the third-party to speak may be controlled by a real-time context monitoring (RTCM) service operative to monitor the ongoing session. In some arrangements, the RTCM service may be configured to notify the third-party when the therapy is ongoing via a suitable UI on the third-party device. In some arrangements, the RTCM service may also mute or otherwise disable the microphone and/or other AV functionalities of the third-party device until the therapy adjustments are complete, as described previously with respect to the process flows of FIGS. 18A-18C. In some arrangements, third-party devices/users may therefore be provisioned with appropriate roles and/or profiles for logging in via the third-party applications, e.g., as family members, caregivers, secondary clinicians, etc., which may be maintained in a database associated with a VC platform implementation, e.g., VC platform 1300, as previously described.

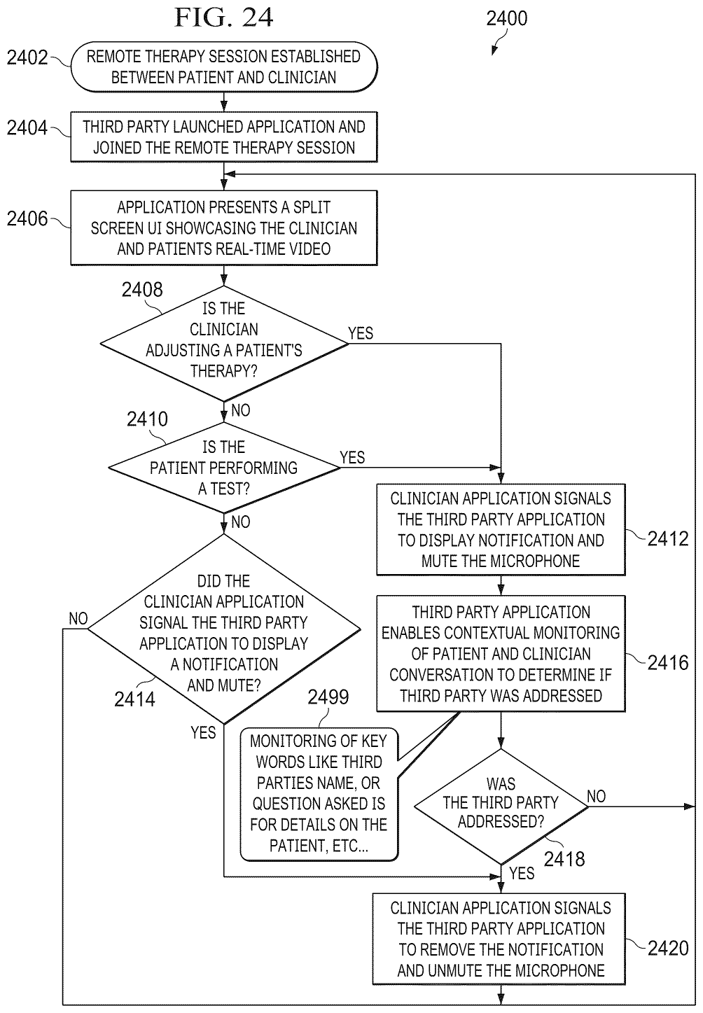

FIG. 24 depicts a flowchart illustrative of blocks, steps and/or acts of a process with additional details for effectuating a management process with respect to an authorized third-party/device enabled to join an ongoing session according to an implementation of the present disclosure. Example process 2400 may commence with establishing a remote therapy session between a patient and a clinician, as set forth at block 2402. A third-party user may launch an application to join the session, which may be effectuated responsive to obtaining approval from one or more principal parties (block 2404). In some example embodiments, the GUI of the third-party device/application may present a split screen that shows the patient's and clinician's real-time A/V session (block 2406). At decision block 2408, a determination may be made whether the clinician is adjusting the patient's therapy. If so, the clinician programmer application may send a control signal to the third-party device/application to display a suitable notification and mute the telephone of the third-party device (block 2412). Responsive to the notification from the CP device, the third-party application may commence/enable contextual monitoring of the conversation between the patent and the clinician to determine if the third-party is invited or otherwise addressed (block 2416). As noted previously, the contextual monitoring of the A/V session may include monitoring the audio track for the occurrence of any triggering or key words or phrases. Such triggering words may include the third-party's personal details (e.g., name, etc.), or a question is asked of the third-party regarding the patient, etc. as exemplified at block 2499. If no triggering words or phrases are detected (decision block 2418), the process flow may return to block 2406, where the third-party continues to be an observer of the session.

Returning to decision block 2408, if the clinician is not adjusting the patient's therapy, a further determination may be made if the patient is performing a test (decision block 2410). If so, the process flow may proceed with blocks 2412, 2416, 2418 as previously discussed. Otherwise, a still further determination may be made if the clinician previously sent a signal to the third-party device application to display a notification and/or caused disabling of the third-party device's microphone (block 2414). If so, the clinician application may generate a signal to the third-party device application to remove the notification and enable the microphone (block 2420). Thereafter, the process flow may return to block 2406. On the other hand, if the clinician did not previously send a signal to the third-party device application to display a notification, the process flow may also return to block 2406. In some example embodiments, if the third-party was addressed and/or certain triggering words were detected in the monitored audio track, the flow may proceed to block 2420 from decision block 2418 for removing the notification and enabling the third-party device's microphone. Thereafter, the process flow may return to block 2406 as noted hereinabove.

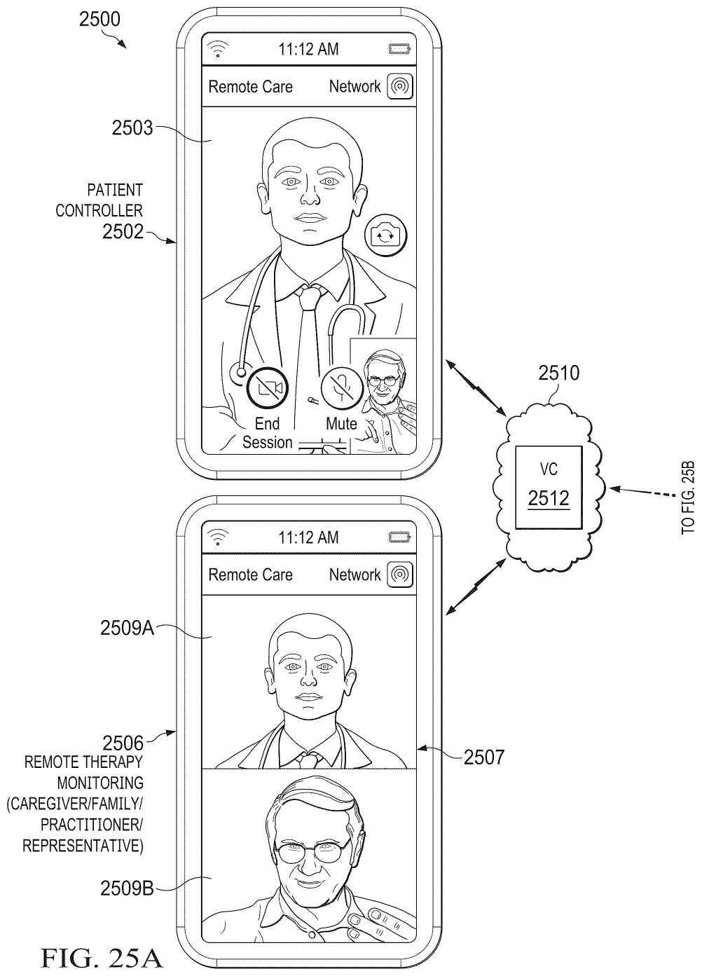

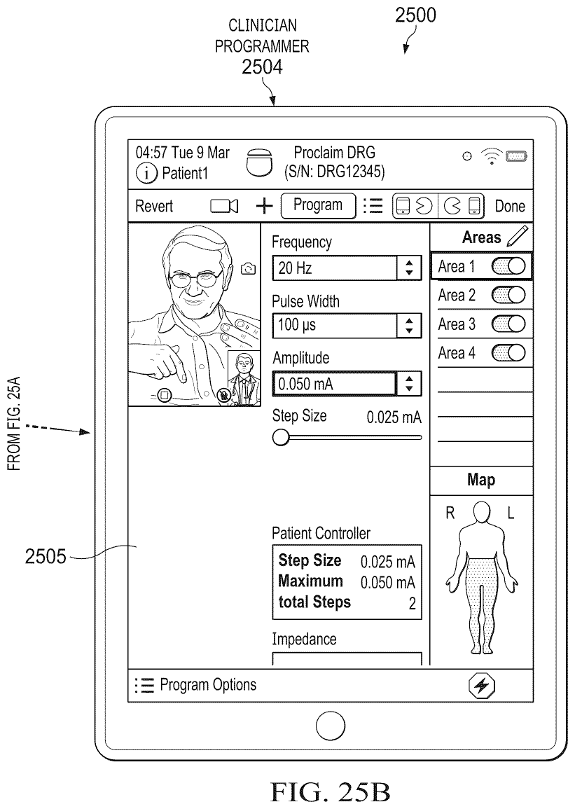

FIGS. 25A and 25B together depict an example network architecture 2500 including a virtual clinic for facilitating third-party enablement with respect to an ongoing integrated remote therapy session according to an implementation of the present disclosure. Example network architecture 2500 is a representative subset of the digital healthcare network environments exemplified in FIGS. 1A/1B and 12, wherein a PC device 2502 and a CP device 2504 are engaged in an ongoing remote care session facilitated by VC platform 2512 deployed in a network 2510. In some example embodiments, PC and CP devices 2502, 2504 may each display normal GUI screen views 2503, 2505, respectively, associated with the remote therapy/programming session. A third-party, using device 2506 and operating with a third-party application, e.g., in conjunction with a remote therapy monitoring service, may be joined according to the methods set forth herein. As illustrated, a GUI screen 2507 associated with the third-party device 2506 may include a split screen view to show both the clinician's video 2509A as well as the patient's video 2509B.

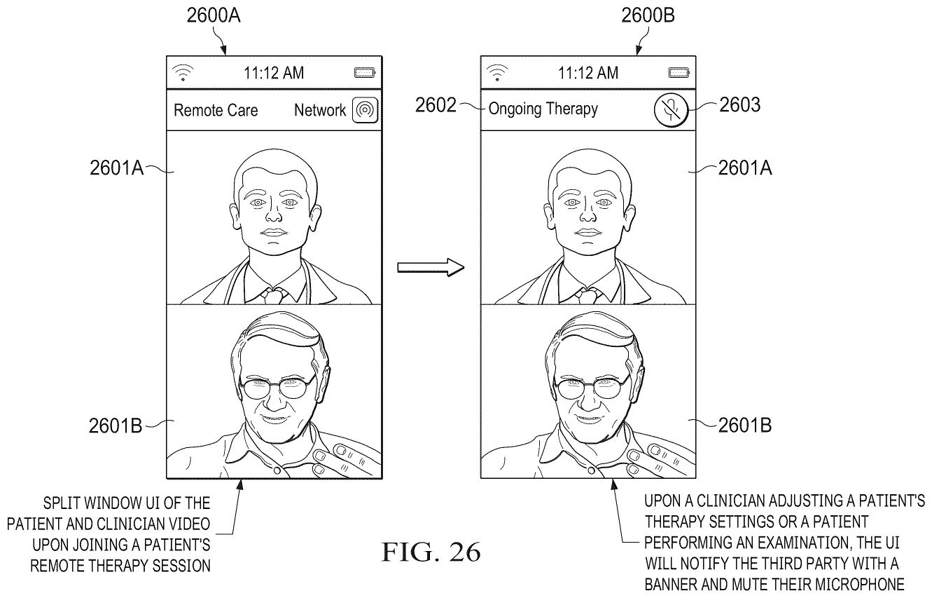

FIG. 26 depicts screenshot views associated with a user interface of a third-party device having a remote monitoring application for joining a remote therapy session according to an implementation of the present disclosure. A split screen GUI 2600A is illustrative of the third-party device upon joining the session, wherein both the clinician's video and the patient's video may be shown simultaneously in separate portions 2601A and 2601B, respectively, similar to the embodiments set forth above. In some arrangements, this split screen view may be presented to the third-party until one of the principal parties withdraws consent to share the A/session. Responsive to the clinician adjusting the patient's therapy, or if the clinician and patient are performing an examination, GUI screen 2600B of the third-party device may be operative to present a suitable notification (e.g., Ongoing Therapy 2602) as well as an icon 2603 indicating the muted microphone.

In some example arrangements where various third parties may be authorized to join an ongoing session between the principal parties (e.g., a patient and a clinician), it is important to ensure that privacy and identity concerns of the principal parties are not compromised, at least for purposes of several regulatory and legislative requirements. Accordingly, some example embodiments may be advantageously configured to effectuate appropriate privacy policy controls in a remote therapy scenario, as set forth previously with respect to example process 1900 of FIG. 19 described above. Depending on a particular deployment scenario, example embodiments may be configured to help comply with individual privacy and data protection laws (e.g., the Health Insurance Portability and Accountability Act (HIPAA), EU's General Data Protection Regulation (EU GDPR), etc.) that may be applicable to various pieces of data, e.g., PAD 1250 exemplified in FIG. 12. Some example embodiments may also be configured with the functionality to record/store A/V sessions joined by third parties without privacy concerns, where such recorded sessions may be used for human training as well as ML/AI training. In still further variations, example embodiments may be provided with the functionality to configure or define suitable privacy controls with respect to different third-party actors as well as relative to the principal parties, as will be set forth below in additional detail with respect to some examples herein.

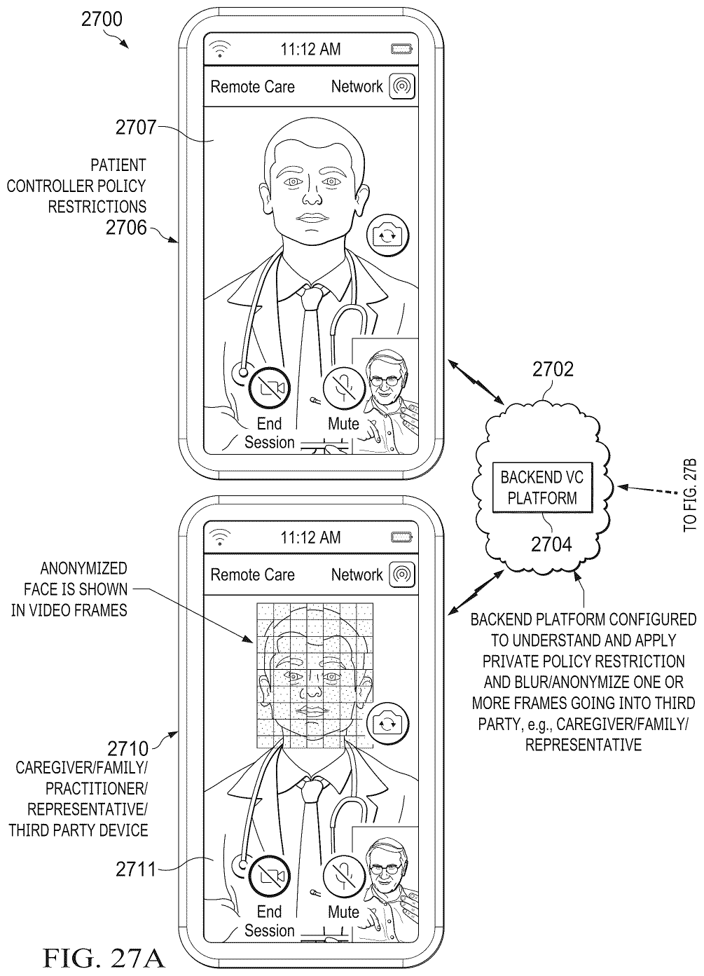

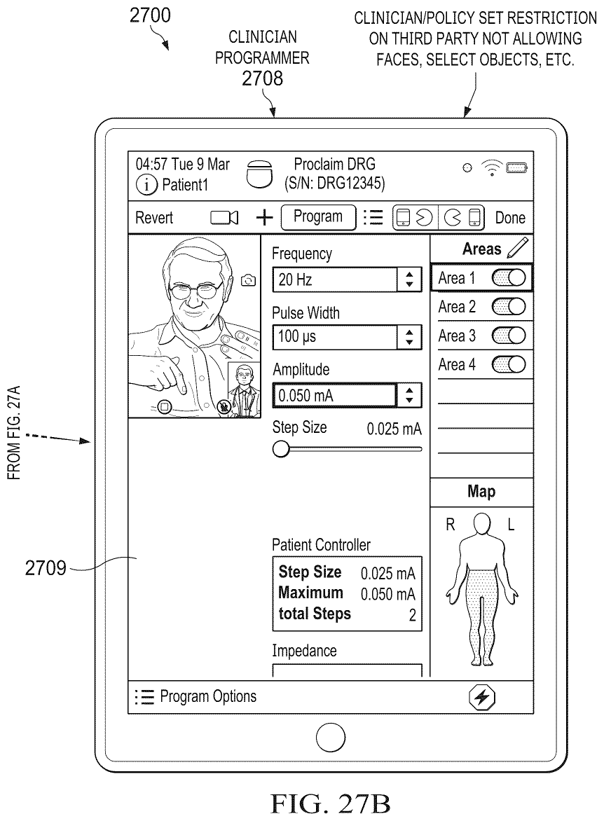

FIGS. 27A and 27B together depict an example network architecture including a virtual clinic for facilitating privacy control with respect to third parties joining an ongoing integrated remote therapy session according to an implementation of the present disclosure. Similar to the network architecture examples described above, network architecture 2700 is a representative subset of the digital healthcare network environments exemplified in FIGS. 1A/1B and 12, wherein a PC device 2706 and a CP device 2708 are engaged in an ongoing remote care session facilitated by a backend VC platform 2704 deployed in a network 2702. In some example embodiments, PC and CP devices 2706, 2708 may each display normal GUI screen views 2707, 2709, respectively, associated with the remote therapy/programming session. A third-party device 2710 operating with a third-party application, e.g., in conjunction with a remote therapy monitoring service, may be joined according to the methods set forth herein. In some arrangements, a GUI screen 2711 associated with the third-party device 2710 may include a split screen view to show both the patient's and clinician's video, although it is not a requirement for purposes of at least some embodiments herein.

In one implementation, either of the principal actors, e.g., the patient and/or the client, may set restrictions on whether the joined third-party can see the principal's facial images, or images of the background of their respective surroundings including any personal objects that may have or provide hints of the respective principal's identity and/or location, etc. In one implementation, such restrictions may be dynamically configured, e.g., on a session-by-session basis, on a third-party-by-third-party basis, on a therapy by therapy basis, etc. In one implementation, restrictions may be preconfigured and stored in a network node/functionality associated with VC platform 2704. Further, depending on the therapy type, some sessions may require fewer or more restrictions and privacy controls. Regardless of how the privacy restrictions are configured, example VC platform 2704 may be configured with the functionality to understand and apply suitable private policy controls and restrictions with respect to the images/frames being shared with the third-party device 2710. As previously noted, techniques such as data anonymization and/or image blurring may be implemented, locally, remotely and/or both, to “sanitize” the frames of a shared A/V session provided to the third-party.

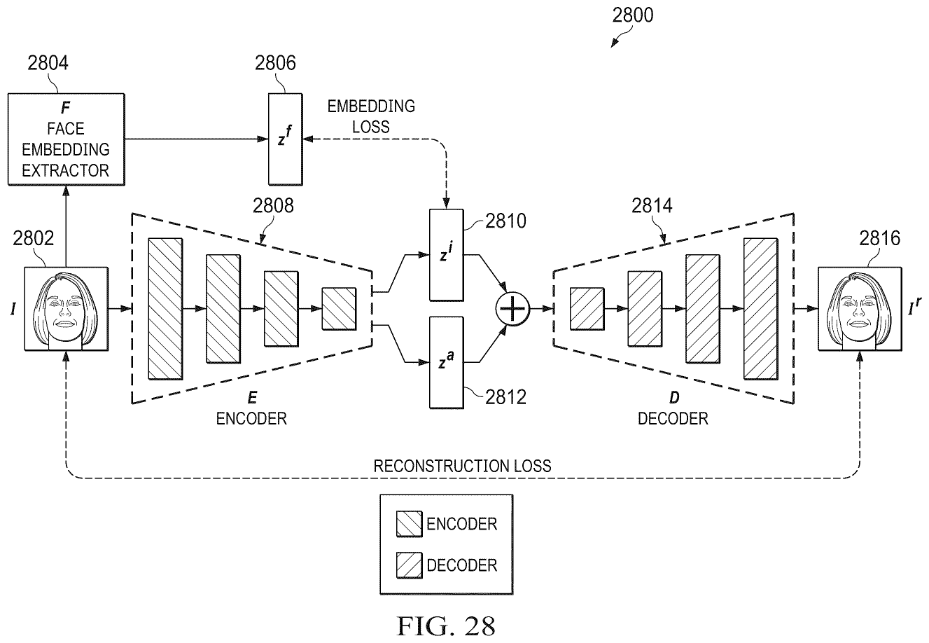

FIG. 28 depicts an example neural network implementation or apparatus 2800 for anonymizing patient/clinician images and data with respect to an A/V session shared with a third-party according to an embodiment of the present disclosure. In one arrangement, apparatus 2800 may include one or more trained neural networks with respect to both encoder and decoder portions 2808, 2814, wherein the neural network(s) may apply data and variant factors to generate different sets of image data. A face embedding extractor module 2804 may be applied in conjunction with an input image 2802. A reconstruction loss may be configured with respect to image 2802 and a decoded image 2816, wherein an embedding loss generated by Z′ block 2806 may be provided as an input to Z block 2810. Output of the encoder neural network 2808 may also be provided as an input to both Zⁱ block 2810 and Z^{a} block 2812. It should be appreciated that although a particular neural network implementation is set forth herein for image blurring, other techniques may also be implemented in additional and/or alternative embodiments of the present disclosure.

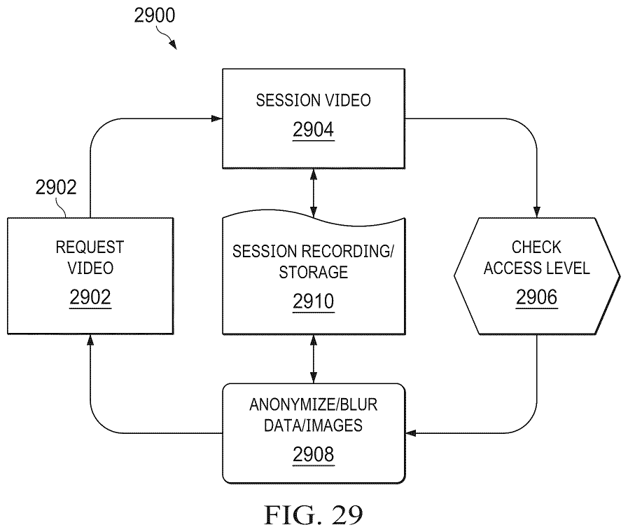

FIG. 29 depicts an example schematic architecture for facilitating recording/storing of a remote therapy session wherein an A/V session may be shared based on privacy policy control according to an embodiment of the present disclosure. Schematic architecture 2900 may involve various functional stages/states disposed in an interoperating mechanism, wherein a request for video (block 2902) may be made by an entity (e.g., a network entity and/or a principal party, etc.) with respect to a shared video session (block 2904). An access level and/or privacy restriction level may be checked against a third-party that has been granted permission to join the A/V session (block 2906). If the access level is not sufficient, e.g., based on a policy management mechanism, the image data may be anonymized (block 2908). A session recording database 2910 provided as part of a VC platform (e.g., VC platform 1300 shown in FIG. 13) may record/store the anonymized images, for example, in lieu of the actual images transmitted between the principal parties (e.g., the patient and the clinician) in some example implementations.

In some example implementations of the present disclosure, a digital healthcare infrastructure may therefore be configured to provide one or more of the following: (i) defining privacy control for every person joining during a VC session; (ii) blur/mask/anonymize the facial features of individuals who have not given applicable permissions with respect to all or portions of the contents of video frames; (iii) blur/mask/anonymize defined objects at a patient's and/or clinician's location (e.g., including background equipment and display monitors that may show patients' data, background artwork or personal effects in a residential location, etc.); (iv) reducing the digital display “clutter” seen by the clinicians in an A/V session, e.g., background object images, thereby helping the clinicians focus solely on the patient without distraction; and (v) record/store the shared session using data/image anonymization techniques to avoid any privacy and/or data protection concerns. In still further example arrangements, an implementation may be configured to present various polices regarding privacy levels, object anonymization, person anonymization, etc. to an application's GUI of the clinician when the clinician launches a Remote Generator window (e.g., Generator window 505 described previously with respect to the embodiments of FIGS. 5A and 5B). In some arrangements, the clinician programmer application may be provided with the functionality to present the clinician a plurality of selection options to set/reset privacy control levels for one or more persons joining a virtual clinic session. Example privacy control levels may include information relating to, without limitation, the persons, objects, facial features, face anonymizers, background blurring, and the like.

Set forth below are some additional and/or alternative embodiments including further details with respect to enabling third parties to join virtual care (VC) or remote care (RC) services and sessions according to the teachings herein. Skilled artisans will recognize upon reference hereto that example third-party enablement embodiments are particularly advantageous in various deployment scenarios. For example, if the patient needs the presence, assistance and/or services(s) of another person or a family member during a remote care session with a clinician (or other remote healthcare provider, etc.), which may initially be established as a two-party session according to some examples set forth in detail above, it would be beneficial to configure a system where the endpoint device (such as, e.g., device 700 illustrated in FIG. 7 described above) that is operated by the other person or family member is operable to share the A/V content stream of the session in accordance with some representative embodiments set forth previously in order that the participation by the third-party user is valuable to the fullest extent possible. If the patient and the clinician do not speak the same language, in an example scenario, absence of an interpreter in the session can render the RC/VC session less advantageous, especially when compared against the in-office session scenarios where interpreters may be readily available to be called upon as and when needed. In another example scenario, if the clinician wants an expert opinion from another clinician during an RC/VC session, it would be advantageous to configure a system that allows the third-party device to have appropriate levels of authorization for facilitating collaborative medical care, including, e.g., providing treatment via remote IMD programming.

In some examples, a suitable app may be provided with an example endpoint or edge device for effectuating one or more aspects of device functionality with respect to establishing RC/VC sessions via a network-based platform, e.g., VC platform 1214, as previously set forth. As further noted, the edge device app may comprise the myPath™ app that may be operative in conjunction with additional apps, e.g., a VC app, which together may be configured to effectuate, or operate as part of, a network-based digital healthcare infrastructure or ecosystem that may be exemplified by the NeuroSphere™ system from Abbott Labs in some example implementations. In accordance with the teachings herein, an example device app may be adapted to facilitate a valid user (e.g., a patient, physician, authorized care administrator, etc.) for onboarding a third-party user and associated device into the system by providing the third-party user with suitable access, e.g., ephemeral (which may be configurable) or permanent access to specific data and/or actions during or outside an RC/VC session. Some examples hereinbelow may therefore be directed to a method and associated message flows for accomplishing a third-party onboarding process in a digital healthcare infrastructure. Some further examples may be directed to methods and systems for optimizing the interaction within a video session by prioritizing video framing and data traffic dynamically, e.g., based on the therapy delivery state, role of the user in a three-party RC/VC session, etc. Some further examples may be directed to embodiments configured to provide or facilitate dynamic temporal and/or persistent authorization grants for specific service providers based on, e.g., hierarchical levels, roles, entitlements, relations, and other relevant parameters.

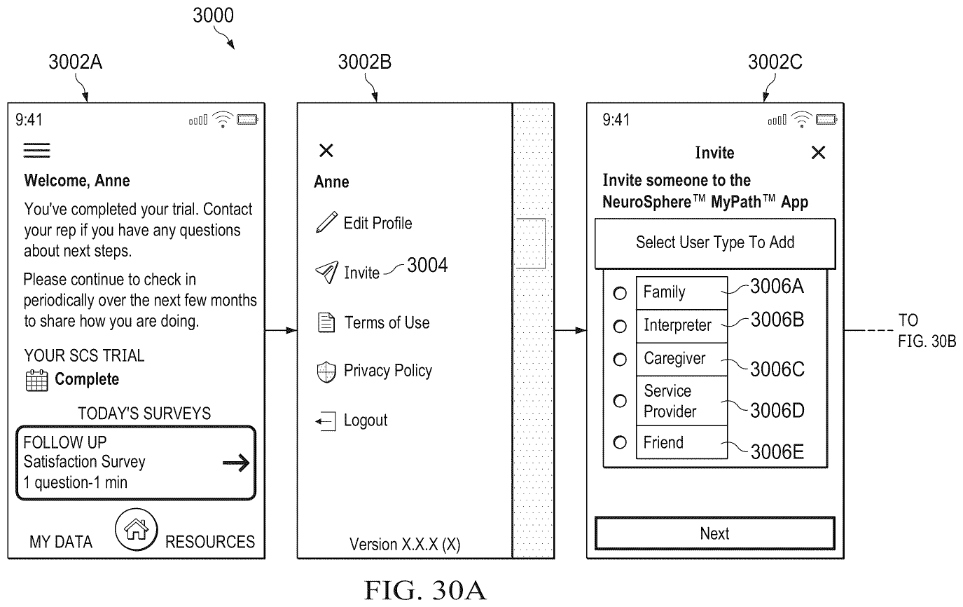

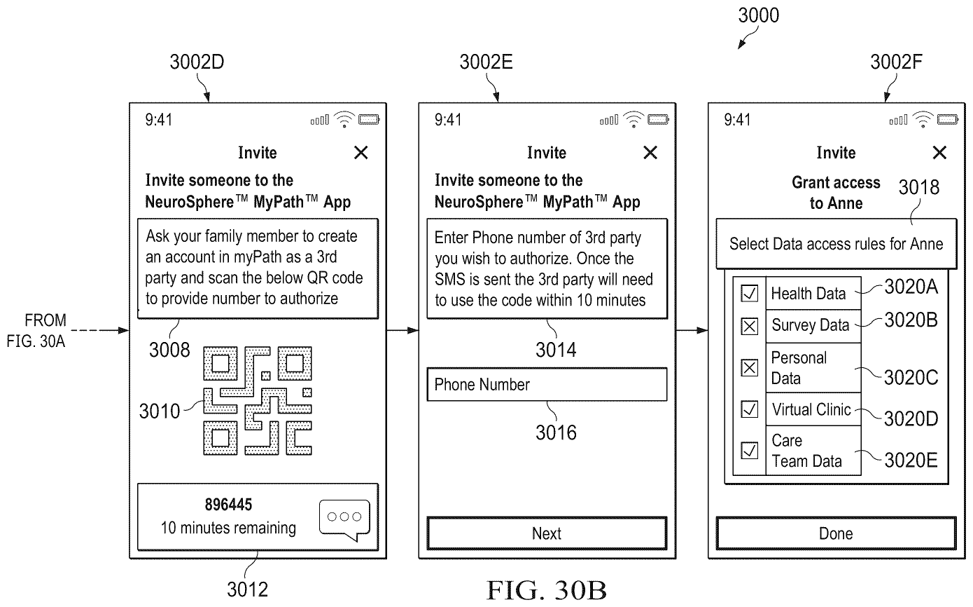

Turning to FIGS. 30A and 30B taken together, depicted therein is a sequence 3000 of a plurality of UI screenshots or representative display screens 3002A-3002F of a patient's device that may be effectuated for inviting and providing authorization to a third-party user according to an example embodiment of the present disclosure, where the various UI display screens may be effectuated in response to appropriate code portion(s) being executed by a device processor, e.g., processor 806 under the control of a patient controller application, e.g., application 802 shown in FIG. 8. By way of example, display screen 3002A is illustrative of a scenario where the patient has completed a therapy (e.g., a trial therapy) and is provided with a dialog box that may include a window configured to display, inter alia, a menu option 3004 for inviting/onboarding a third-party user, e.g., as illustrated in the display screen 3002B. In an example implementation, the patient may be allowed to select the type of user that will be invited for onboarding in a digital healthcare infrastructure system, such as, e.g., NeuroSphere™ system using the myPath™ app, as exemplified in the display screen 3002C. A variety of user types may be configured, e.g., Family 3006A, Interpreter 3006B, Caregiver 3006C, Service Provider 3006D, and Friend 3006E, although other user types may be defined for different patients depending on a particular deployment scenario. In one arrangement, various user types may be configured with appropriate roles and authorization levels including, e.g., data access levels, actions that can be undertaken by the third-party user, etc., which may depend on geographical location(s) of the third-party user, hierarchical relations, entitlements, temporal/persistent nature of authorization, and the like. In some arrangements, access levels, priority levels, etc. may be preconfigured as part of third-party role-based profiles at a network node, e.g., VC/RC platform 1214 of the infrastructure shown in FIG. 12.

In response to selecting a particular user type, the patient may be required to contact the third-party user as illustrated in the display screen 3002D to provide suitable credentials for facilitating secure onboarding of the party. In some arrangements, an in-band or out-of-band communication scheme such as, e.g., texting, directing messaging (DM) through social media platforms such as Twitter, Instagram, and Facebook, etc., or email, and/or the like, may be effectuated with the third-party user, wherein one or more security/validation credentials (e.g., time-stamped quick response (QR) codes, (pseudo) random (alpha) numeric sequences or numbers, etc., that may have an valid time widow associated therewith within which a response needs to be entered) may be included, as exemplified by dialog box portions 3008, 3010, 3012 of the display screen 3002D.

In an example implementation, a phone number of the third-party user may be entered by the patient, as exemplified by dialog box portions 3014, 3016 of the display screen 3002E. The patient may also be required to select appropriate data access rules that specify the type of patient data that may be accessed by the onboarding third-party user, as exemplified by dialog box portion 3018 of the display screen 3002F. In some example implementations, the type of data that can be accessed by a third-party user may be role-dependent, which may be preconfigured. In some example arrangements, a menu of data types may be provided to the patient that may be selected or deselected dynamically by the patient for specifying the data access/type levels. As exemplified in the display screen 3002F, data types may comprise, without limitation, Health Data 3020A, Survey Data 3020B, Personal Data 3020C, VC Data 3020D, and Care Team Data 3020E, at least of some which may comprise a portion of PAD data 1250 described above in reference to the architecture shown in FIG. 12. As exemplified herein, access to Health Data 3020A, VC Data 3020D, and Care Team Data 3020E is granted while access to the other data types, Survey Data 3020B and Personal Data 3020C is denied.

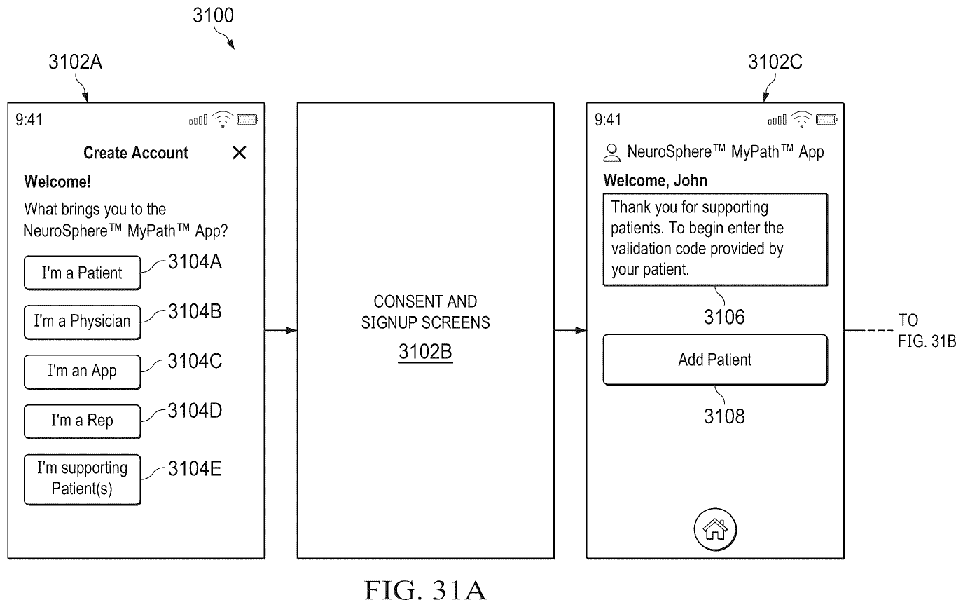

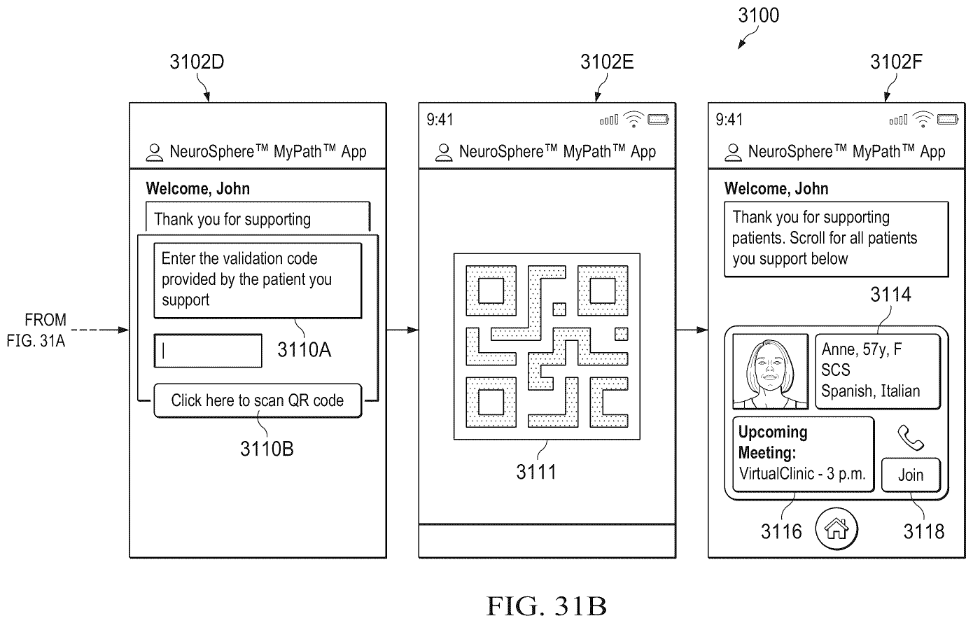

FIGS. 31A and 31B together depict a sequence 3100 of a plurality of UI screenshots or representative display screens 3102A-3102F of an invited third-party's device that may be effectuated for facilitating login, registration and authorization of the invited third-party user according to an example embodiment of the present disclosure, where the various UI display screens may be effectuated in response to appropriate code portion(s) being executed by a device processor, e.g., processor 702 under the control of an application, e.g., application 708-N shown in FIG. 7. In one arrangement, the invited third-party user may download the application, which, in response to launching, may present an initial display screen 3102A that allows creating an account and registering with the digital healthcare infrastructure associated with the downloaded application. In one arrangement, the initial display screen 3102A may also present a plurality of options as to facilitating a self-identified role designation of the third-party user, such as, e.g., Patient 3104A, Physician/Clinician 3104B, AI-Bot/Application 3104D, Service Representative 3104D, and Patient Support Provider 3104E, although various other roles may be defined depending on implementation. Responsive to selecting a particular role, e.g., Patient Support Provider 3104E, applicable display screens may be effectuated for e-consent, acceptance of terms of use, etc., as well as user setup/signup, which are collectively shown at reference numeral 3102B.

With respect to supporting one or more patients, a third-party user may be required to respond to one or more dialog boxes for adding one or more patients, as exemplified by dialog boxes 3106, 3108 of display screen 3102C. Responsive to selecting Add Patient dialog box 3108, the third-party user may be required to provide a validation code, e.g., a QR code, an alphanumerical or number code, or other type of indicia, that have been provided by the supported patient in a manner as set forth above in an example implementation. Skilled artisans will recognize upon reference hereto that some of the foregoing processes may be implemented in conjunction with additional challenge-response authentication schemes such as, e.g., CAPTCHA, etc., in some examples. Further, some of the processes set forth in FIGS. 30 and 31 can be generally asynchronous although there a temporal validation process based on validation code transmission and reception that may couple the two sequences in some examples. Still further, the downloading of the app and initial signup stages as exemplified by the display screens 3102A/3102B may be asynchronous with, or otherwise decoupled from, from the process of adding supported patients. In other words, in an example scenario, a third-party user may download the application and set up the user account etc., and then wait until a patient desiring to add the third-party user sends the validation code(s) via a suitable mechanism as previously described. After receiving the validation code(s), the third-party user may bypass some of the initial account setup/signup steps and launch the application to be taken directly to a dialog box or screen for adding the patient(s) that has/have transmitted the validation code(s) providing authorization. Accordingly, it should be appreciated that there can be several permutations and combinations of how the sequences of an invited third-party user and a requesting patient can be rendered interoperable depending on the architecture of a VC/RC platform and associated app interactivity.

In an example implementation, one or more additional dialog boxes 3110A/3110B may be presented to the third-party user in response to selecting the Add Patient option 3108, wherein the third-party user may be required to enter one or more validation codes previously received from the requesting/authorizing patient, as exemplified in display screen 3102D. By way of illustration, a QR code is entered, e.g., QR code 3111, as shown in display screen 3102E, whereupon the third-party user may be provided with a pictorial indicium such as a thumbnail photo of the patient 3114, indicating the personal identification data as well as demographic data and health/treatment data of the patient such as, e.g., the name, gender, age, ethnicity/race, geolocation, languages spoken, IMD treatments (e.g., SCS, DBS, etc.), among others, as exemplified by patient identity display portion 3114 shown in display screen 3102F. Further, additional dialog boxes 3116, 3118 may be provided to indicate any upcoming sessions scheduled with the patient as well as a radio button to join a selected session at the scheduled time. In some arrangements, the third-party user may be provided with additional options such as, e.g., adding the upcoming meeting schedule(s) to a calendaring application, a video teleconferencing application, exporting/redirecting to another display device, etc., without limitation.

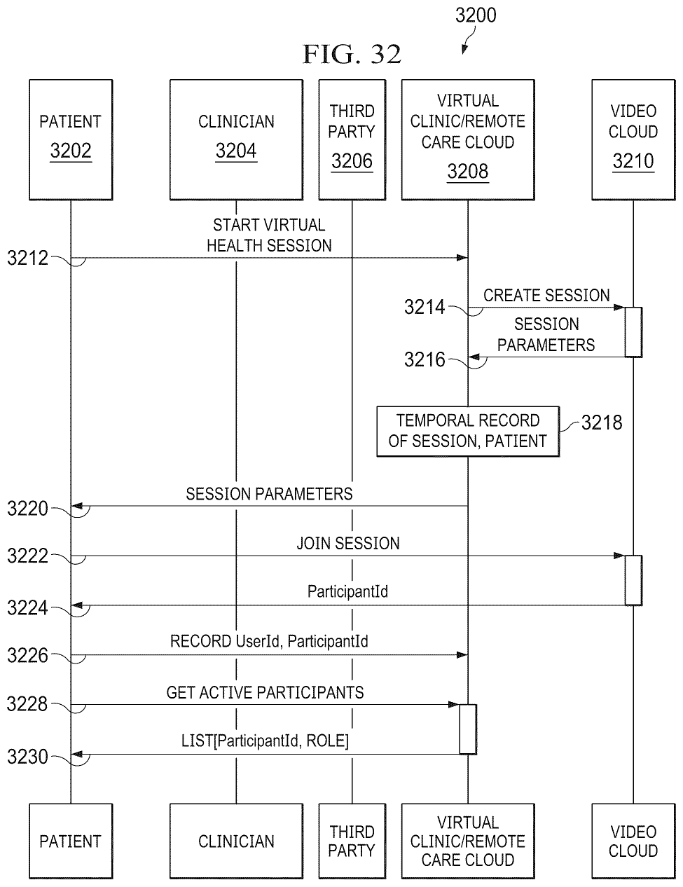

FIG. 32 depicts a message flow 3200 associated with initiating a multi-party session according to an example embodiment of the present disclosure. In one arrangement, at least some portions of the message flow 3200 may involve interactions among a patient/device 3202, a clinician/device 3204, a third-party user/device 3206, a cloud-based VC/RC platform 3208 and a cloud-based A/V media delivery/distribution (MDD) network 3210, or a subset thereof, which may be initiated by the patient 3202 generating a request 3212 to the VC/RC platform 3208 for starting a VC/RC health session. In response, a media session creation request 3214 may be transmitted by the VC/RC platform 3208 to a suitable session manager of the MDD network 3210, which may involve adaptive bitrate (ABR) streaming in some applications. Responsive thereto, media session parameters 3216 may be obtained by the VC/RC platform 3208. In one arrangement, the VC/RC platform 3208 may be configured to record the VC/RC session with the patient, as exemplified by block 3218, which may be executed in a manner roughly similar to the schematic architecture 2900 described above in reference to FIG. 29. Regardless of whether a session is to be recorded/stored, the session parameters may be transmitted by the VC/RC platform 3208 to the patient/device 3202 via suitable messaging 3220. Thereafter, a media session may be established via the MDD network 3210 that may be joined by the patient 3202, as indicated by reference numeral 3222, whereupon a ParticipantID may be provided to the patient 3202 via a message 3224 from the MDD network 3210. In response, the patient 3202 may provide a UserID and ParticipantID to the VC/RC platform 3208 via a message 3226. The patient 3202 may also generate a message 3228 to the VC/RC platform 3208 to obtain a list of participants, e.g., the clinician 3204, one or more third-party users 3206, etc., that may need to be added to the session. In response thereto, the VC/RC platform 3208 may be configured to provide the list of requested participants and respective role designations to the patient 3202 via suitable messaging 3230.

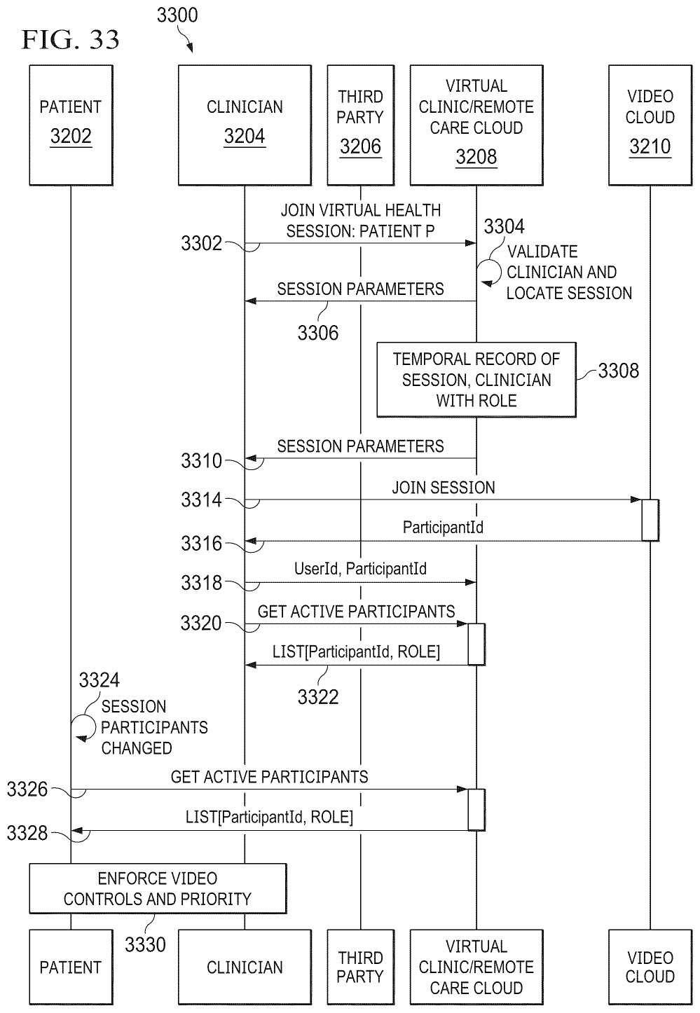

FIG. 33 depicts a message flow 3300 associated with adding a health provider, e.g., clinician 3204, to a multi-party session according to an example embodiment of the present disclosure. In one arrangement, the clinician 3204 may generate a request 3302 to the VC/RC platform 3208 for joining a VC/RC health session with the patient 3202. The VC/RC platform 3208 may be configured to validate the clinician 3204 as indicated by validation flow 3304, which may be executed in a number of ways in accordance with example embodiments set forth hereinabove. Responsive to validating the clinician 3204, the session parameters may be provided to the clinician 3204 via suitable messaging 3306. Similar and/or further to the recording of a session with the patient 3202, the VC/RC platform 3208 may initiate and/or continue recording of the session including the clinician 3204 and clinician's role, as indicated by reference numeral 3308. In some arrangements, additional session parameters may be optionally provided, as indicated by message 3310. Thereafter, the clinician 3204 may join the session via the MDD network 3210, as indicated by reference numeral 3314, whereupon a ParticipantID may be provided to the clinician 3204 via a message 3316 from the MDD network 3210. Similar to the messaging flow from the patient 3202 described above, the clinician 3204 may provide a UserID and ParticipantID to the VC/RC platform 3208 via a message 3318. Likewise, the client 3204 may also generate a message 3320 to the VC/RC platform 3208 to obtain a list of participants in an example implementation. Where additional participants are selected by the clinician 3204, e.g., as indicated by messages 3320, 3322, the patient 3202 may be made aware of a change in the participants, if any, as indicated by reference numeral 3324. In an example implementation, the patient 3202 may obtain a list of additional/different participants to be added to the session, as indicated by messages 3326, 3328. The patient 3202 may also selectively/optionally enforce, independently or in combination with the clinician 3204, various A/V controls and assign priorities with respect to the (modified) list of participants based on their respective roles, etc., indicated by block 3330, which may involve video frame control, anonymizing, muting A/V, and the like, as set forth in the present disclosure.

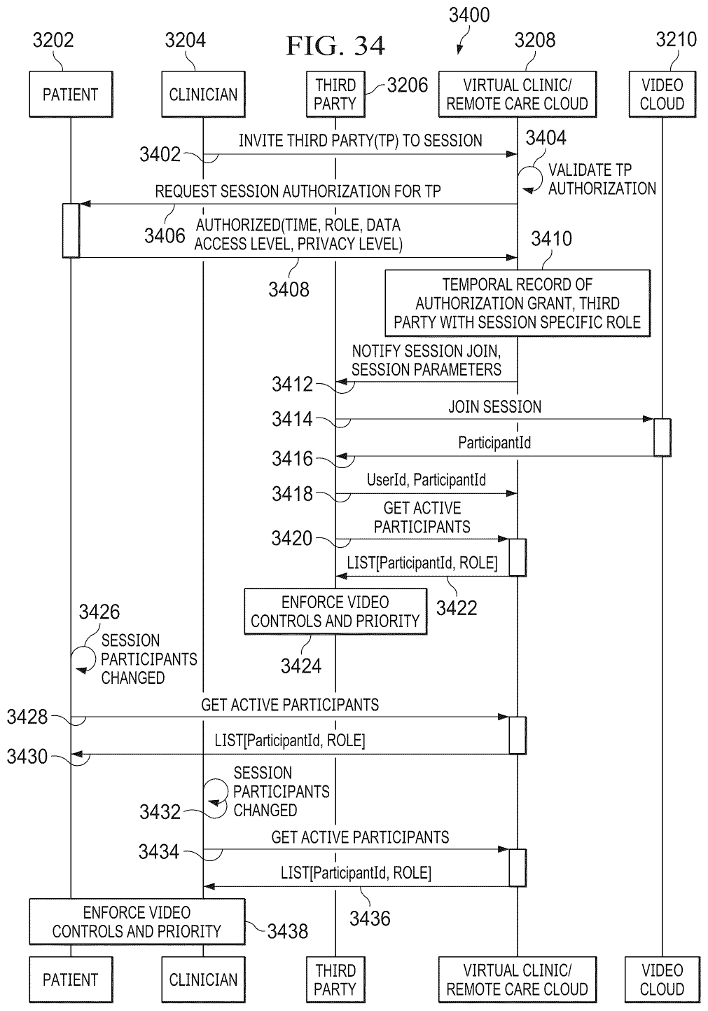

FIG. 34 depicts a message flow 3400 associated with adding a third-party user, e.g., user 3206, to a multi-party session according to an example embodiment of the present disclosure, wherein the third-party user 3206 is invited to join the session by a particular principal, e.g., the clinician 3204. As illustrated, the clinician 3204 may generate a request 3402 to the VC/RC platform 3208 to invite the third-party user 3206, whereupon the VC/RC platform 3208 may execute a validation process 3404 to ensure that the third-party user 3206 is not an unscrupulous party. Responsive to determining that the third-party user 3206 is a valid user, the VC/RC platform 3208 may generate a request 3406 to the patient 3202 to provide authorization for the third-party user 3206. In response, the patient 3202 may provide a grant of authorization 3408, including appropriate role, access level, privacy level, and other suitable controls, to the VC/RC platform 3208. In an example arrangement, the VC/RC platform 3208 may initiate/update the recording of the session including the third-party user and its role, as indicated by block 3410. A session notification message 3412 may be provided to the third-party user 3206, which may include session ID, parameters, etc. In response, the third-party user 3206 may join the session via the MDD network 3210, as indicated by reference numeral 3414, whereupon a ParticipantID may be provided to the third-party user 3206 via a message 3416 from the MDD network 3210. Similar to the messaging flow from the messaging flows described above, the third-party user 3206 may provide a UserID and ParticipantID to the VC/RC platform 3208 via a message 3418. The third-party user 3206 may also generate a message 3420 to the VC/RC platform 3208 to obtain a list of participants in an example optional implementation. Where additional participants are selected by the third-party user 3206, e.g., as indicated by messages 3420, 3422, the third-party user 3206 may enforce A/V controls and assign priority levels (block 3424), e.g., depending on the delegated authority in some implementations. Similar to the selection of third-party users by the clinician 3204, if any, the patient 3202 may be made aware of a change in the participants if selected by the third-party user 3206, as indicated by reference numeral 3426. In an example implementation, the patient 3202 may obtain a (modified) list of additional/different participants to be added to the session, as indicated by messages 3428, 3430. In an example implementation, the client 3204 may also be made aware of any change in the participants, as indicated reference numeral 3432, whereupon the clinician 3204 may obtain a (modified) list of additional/different participants being added to the session, as indicated by messages 3434, 3436. The patient 3202 may also selectively/optionally enforce different A/V controls and priority levels with respect to the (modified) list of participants, e.g., independently or in combination with the clinician 3204, as indicated by block 3438, similar to the processes previously set forth.

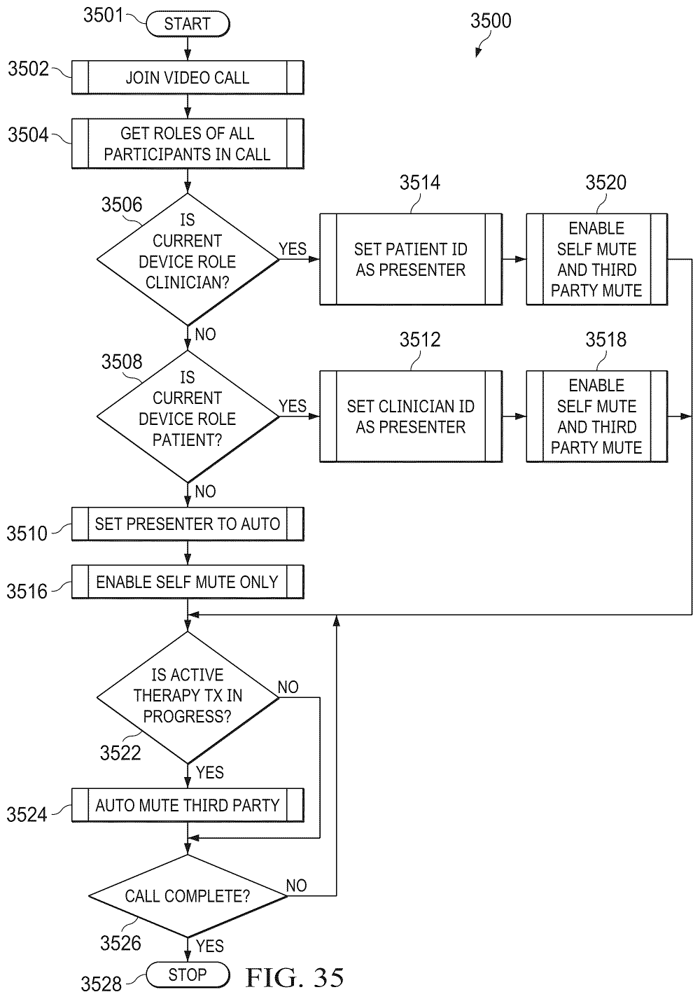

FIG. 35 depicts a flowchart illustrative of blocks, functions, steps and/or acts of a process for providing role-based user interface (UI) control of an A/V session according to an example embodiment of the present disclosure, wherein a portion of the blocks, functions, steps and/or acts may be combined with one or more of other flowcharts of the present disclosure in some implementations. In one arrangement, example process 3500 may start (block 3501) on an edge device when the device joins a VC/RC health session involving a video session. Upon joining the session (block 3502), the roles of all participating entities may be obtained (block 3504). A number of determinations may be made as to whether the role of the device is that of a patient or a clinician, e.g., as set forth at decision blocks 3506, 3508. If the party associated with the device has a patient's role, the patient's ID is set as the presenter (block 3514). Likewise, if the party associated with the device has a clinician's role, the clinician's ID is set as the presenter (block 3512). Otherwise, the presenter's UI control set to auto mode (block 3510) having limited functionality, which may be controlled by other parties in the session, e.g., the principals such as the patient and/or the clinician, in some arrangements. By way of example, in the auto mode, the party (typically, a third-party user invited to join the VC/RC health session) may only be enabled to self-mute the audio channel associated with the session (block 3516). Otherwise, with respect to the principal parties, the device UI may have controls for self-muting as well as muting/terminating the third-party user's device, as exemplified by blocks 3518 and 3520. Example process 3500 may also involve a determination (block 3522) as to whether active therapy is in progress (e.g., adjusting one or more parameters of a stimulation program, etc.). If so, the third-party device may be muted (block 3524) until the therapy is complete. Thereafter, a further determination may be made whether the call (if purely audio) or the session (if both audio and video) is completed (block 3526). If so, the example process flow 3500 with respect to providing role-based UI control on the device is exited (block 3528). On the other hand, if the call/session is still ongoing, the flow control returns to determining whether there is active therapy (block 3522).

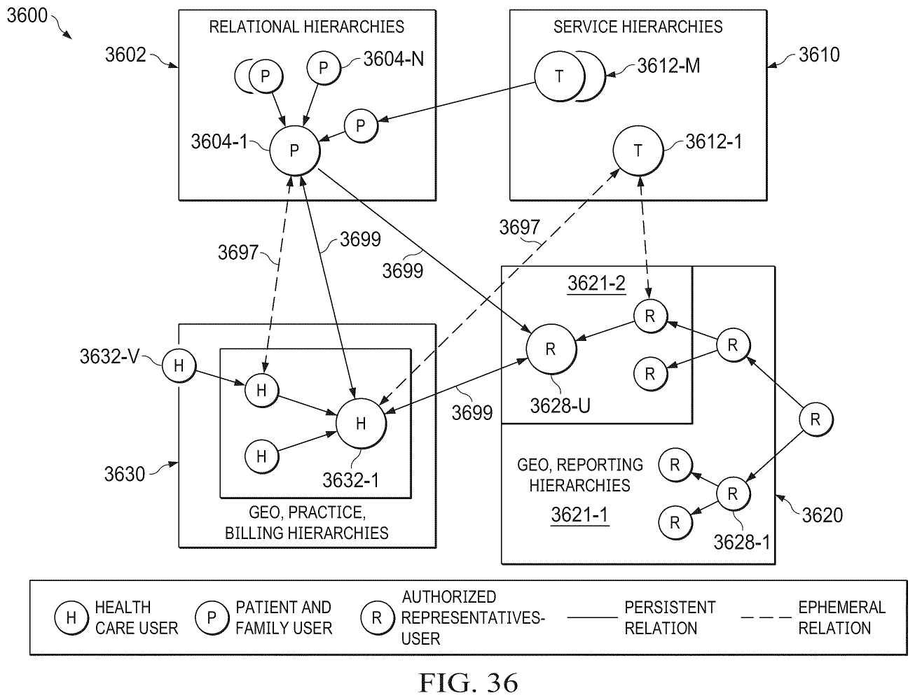

FIG. 36 depicts an example role and hierarchical relationship (RHR) scheme 3600 for assigning different temporal or persistent relations with respect to multiple parties in a multi-party session according to an example embodiment of the present disclosure. Skilled artisans will recognize that at some portions of an example RHR scheme 3600 may be dynamically configured (e.g., during or pursuant to an ongoing multi-party session) in some example implementations. On the other hand, some portions of the RHR scheme 3600 may be preconfigured by way of user and role profile setting at a network node of a digital healthcare infrastructure, e.g., VC/RC platform node 1214 that may be realized as a computer platform 1300 in some arrangements, as previously set forth. Various categories or classes of parties may be defined, e.g., healthcare users, patient and family users, authorized representatives, etc., wherein entities/parties within different classes may be related in a persistent manner or in a temporally limited manner (e.g., ephemeral relationships that may last for just one session, multiple sessions, or over a preconfigured timeframe, etc.). Further, entities/parties among different classes may also be related in persistent or ephemeral relationships. Moreover, entities/parties in different classes may comprise different hierarchies such as, e.g., relational hierarchies 3602 for patient and family users 3604-1 to 3604-N, service hierarchies 3610 for therapy service entities 3612-1 to 3612-M, geolocation and/or reporting hierarchies 3620 for authorized representative users 3628-1 to 3638-U, and geolocation, practice and billing hierarchies 3630 for healthcare users 3632-1 to 3632-V. As illustrated, some of the hierarchies, e.g., hierarchies 3620, 3630, may involve partitions based on, e.g., geolocation, wherein each partition may have a subset of the parties/entities of the overall hierarchy. By way of example, partitions 3621-1 and 3621-2 are shown with respect to the hierarchy 3620. Regardless of how a hierarchical arrangement may be defined with respect to the entities/parties, the relationships between them may be configured to be persistent or ephemeral as noted above, as exemplified by solid lines 3699 for persistent relationships and dotted/broken lines 3697 for ephemeral relationships.

Based on the foregoing Detailed Description, it should be appreciated that some embodiments of the present disclosure may be configured to provide a system and method for effectuating selective onboarding of third-party users into a digital healthcare infrastructure ecosystem (e.g., the NeuroSphere™ system from Abbott) by an authorized user of the system. Some embodiments of the present disclosure may be configured to provide a system and method to restrict data access, controls, functions, and services, etc., by proctoring temporal/permanent authorization levels of the user based on the taxonomy of the onboarding user (e.g., the type or class of user) within the ecosystem. Some embodiments of the present disclosure may be configured to provide a system and method to restrict data access, controls, functions and services by proctoring temporal/permanent authorization levels of the user based on the role hierarchy of the onboarding user within the ecosystem. Some embodiments of the present disclosure may be configured to provide a system and method for facilitating the authenticated and authorized 3^{rd} party to join a therapy session via an application, e.g., a non-medical/non-therapy-based application executing on a COTS device. Some embodiments of the present disclosure may be configured to provide a system and method configured at a backend node (e.g., the VC/RC platform described in detail above), and operative in conjunction with clients, to identify temporal and permanent roles of data streams originating from individual participants in a remote therapy session. Some embodiments of the present disclosure may be configured to provide a system and method for effectuating automatic control of participants, e.g., based on role and relationship focus during a remote therapy session involving more than two participants. Relatedly, some embodiments of the present disclosure may be configured to provide a system and method for effectuating automatic control of UI controls based on the state of therapy and role/relationship of the participants during a remote therapy session. Some embodiments of the present disclosure may be configured to provide a system and method for parties (e.g., third-party users) to be discovered by valid users of the digital healthcare infrastructure based on services offered, cost, geography, rating, presence/availability of the third-party users, and the like. In some examples, low cost rating may be a factor in third-party user discovery, e.g., based on the availability of zero-rating, toll-free data, and/or sponsored data, wherein the communications network operator does not charge end users for participating in a multimedia session pursuant to a specific differentiated service application (e.g., as a bundled client data service) in limited or metered data plans of the end users.

In still further examples, some embodiments of the present disclosure may be configured to provide a system and method for allowing a backend system of the digital healthcare infrastructure ecosystem to provide reimbursement based on services rendered by a 3^{rd} party to the users of the digital healthcare infrastructure ecosystem. For example, the digital healthcare infrastructure ecosystem may be configured to designate the payee and payer roles based on relationships and external systems, e.g., customer relationship management databases, salesforce databases, etc. (which may be based on enterprise database systems using SAP and the like).

In still further examples, some embodiments of the present disclosure may be configured to provide a system and method for effectuating service entitlements and preferences based on roles and hierarchical levels of different groups, e.g., practices, clinics, territories, family members, etc. In some examples, systems and methods may be provided for effectuating ephemeral grant of video/data permissions specific to individual therapy sessions, to individual groups (e.g., practices, healthcare teams, sites, territories, family members, etc.), and the like. In some examples, embodiments of the present disclosure may be configured to provide a system and method for effectuating one or more of: (i) geography-based localization/restriction of 3^{rd} party service selection, (ii) load-based localization/restriction of 3^{rd} party service scheduling, and (iii) rating of 3^{rd} party services from multiple users within a single VC/RC call/session and promotion of effective providers.

In still further examples, some embodiments of the present disclosure may be configured to provide a system and method for effectuating assisted control of UI based on authorization grant by one or more valid users of the digital healthcare infrastructure ecosystem. Some embodiments of the present disclosure may be configured to provide a system and method that allows a principal party (e.g., a patient, a clinician or health care provider, etc.) to terminate the third-party services at any particular time during the session. Relatedly, some embodiments of the present disclosure may be configured to provide a system and method that allows a principal party (e.g., a patient, a clinician or health care provider, etc.) to (re) invite the third-party users for joining the services at any particular time during the session.

As discussed herein, a variety of applications (e.g., patient controller app, remote programming/monitoring app, etc.) may be provided to enable patients having implantable medical devices (IMDs) to control operation of or otherwise communicate with the IMDs (such as neurostimulation systems). Although some embodiments have been discussed in terms of neurostimulation systems, any appropriate implantable medical device may be employed according to some embodiments. For example, cardiac rhythm management devices (e.g., cardiac monitors, pacemakers, defibrillators/cardioverters, glucose monitors, insulin pumps, etc.) may be employed according to some embodiments.

The use of graphical user interface (GUI) components for applications for patients with IMDs is well known. Many of the GUI components utilized for general purpose applications (e.g., an email application) are also employed for applications intended to operate with IMDs. For example, GUI components (e.g., a GUI “button”) that respond to user touch are frequently used for IMD-related apps. The use of common GUI components provides an intuitive experience for users of such applications.

However, many patients with IMDs have physical limitations that cause interaction with common GUI components to be challenging. For example, age-related limitations, vision deficiencies, tremor or other motor symptoms may cause interaction with conventional GUI components difficult. Such challenges may frustrate the users and ultimately lead to non-optimal user outcomes if a given patient is unable to properly interact with a respective IMD-related application.

In some embodiments, monitoring of the patient and the patient's interaction with an IMD-related application is performed. In some embodiments, the monitoring, analysis, and automatic adjustment may be performed, in part, using a background service and/or the IMD-related application. One or more GUI components are modified based on the monitoring when one or more patient difficulties are detected.

In some embodiments, touch input is monitored and analyzed to determine whether to modify one or more GUI components in an IMD-related application. In some embodiments, touch input(s) from capacitive-sensitive display are monitored for analysis. In some embodiments, an in-screen fingerprint camera is employed for capturing touch input for analysis.

For example, GUI controls may be optimized for patients with chronic pain or other patients. By monitoring touch input, GUI Controls are capable of assisting selection of GUI components when irregular finger movement or shaking of hand is detected. For example, GUI controls may be modified to expand, collapse, etc., based on and/or auto-adjusting the touch area in some embodiments. In some embodiments, monitoring and analysis of touch input detects where finger is going on the touchable screen of the patient device and auto zooming one or more GUI controls (if appropriate for a given patient).

In some embodiments, touch input is monitored and processed to capture metrics related to movement irregularities or finger shaking and communicate this information to a remote care system to understand progress or improvement of a patient condition.

In some embodiments, software on a patient mobile device uses the device camera to monitor a user's visual gaze, facial kinematics, and facial expression, and the like. The monitoring of such data may estimate distance between the device and the patient for detecting a user struggling to interact. Upon detecting a user squinting, and/or placing the device closer to their eyes, the software determines the GUI location the user is gazing, and automatically adjusts the text size/UI control while monitoring till the user is no longer straining to read the text.

In other embodiments, while in a telehealth/remote programming session, the application or software may utilize NLP (Natural Language Processing) to detect users asking (e.g., “what did you say”, “huh”, or other auditory expressions representative of difficulty hearing or comprehending) the other party speaking to repeat what they just said. Upon detection of such expressions, the software may automatically increase the speaker volume on the patient's mobile device.

In some embodiments, the application or software may also adjust speaker volume based on the user's ear distance from the device and using NLP to detect the user asking the other connected user to repeat what they said.

In some embodiments, an application for patients (with an implantable medical device) utilizes the mobile devices camera to monitor a user's visual gaze, facial kinematics, and facial to mobile device distance for detecting a user struggling to interact with an application in a background service. Upon detecting a user squinting, and/or placing the device closer to their eyes, the service determines the app location the user is gazing, and automatically adjusts the text size/UI control while monitoring till the user is no longer straining to read the text.

In some embodiments, the application may also be configured to adjust the device's speaker volume based on the user's ear distance from the device and using NLP to detect the user asking the other connected user to repeat what they said.

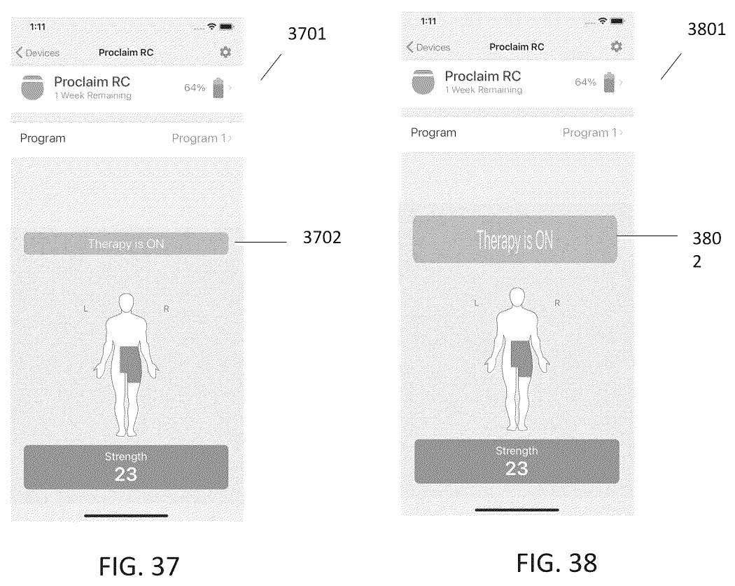

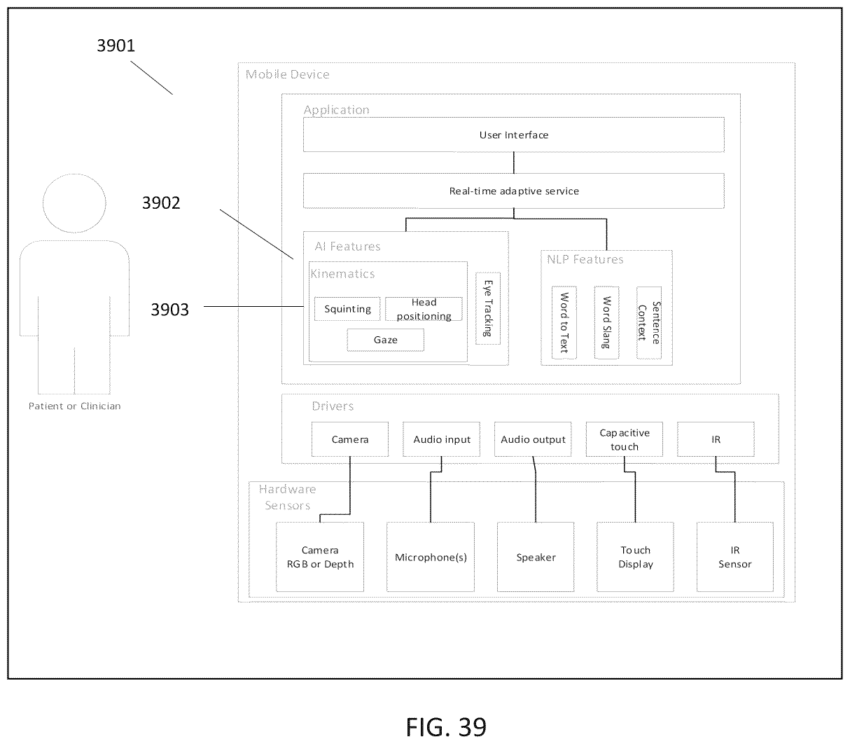

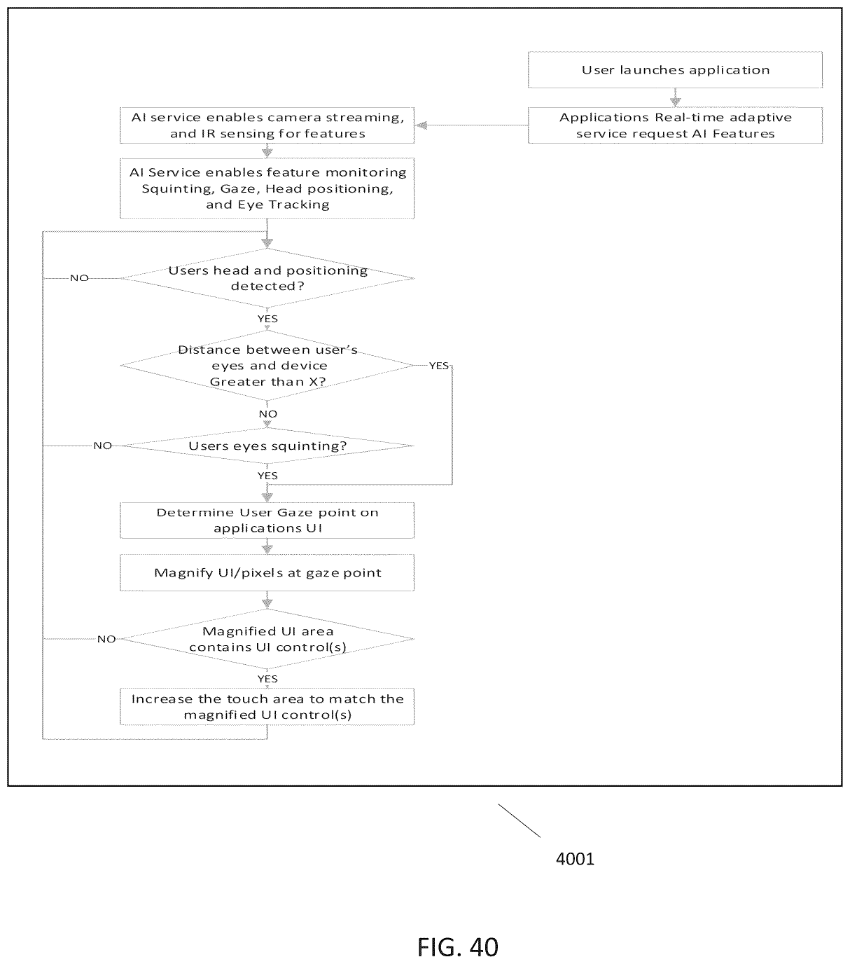

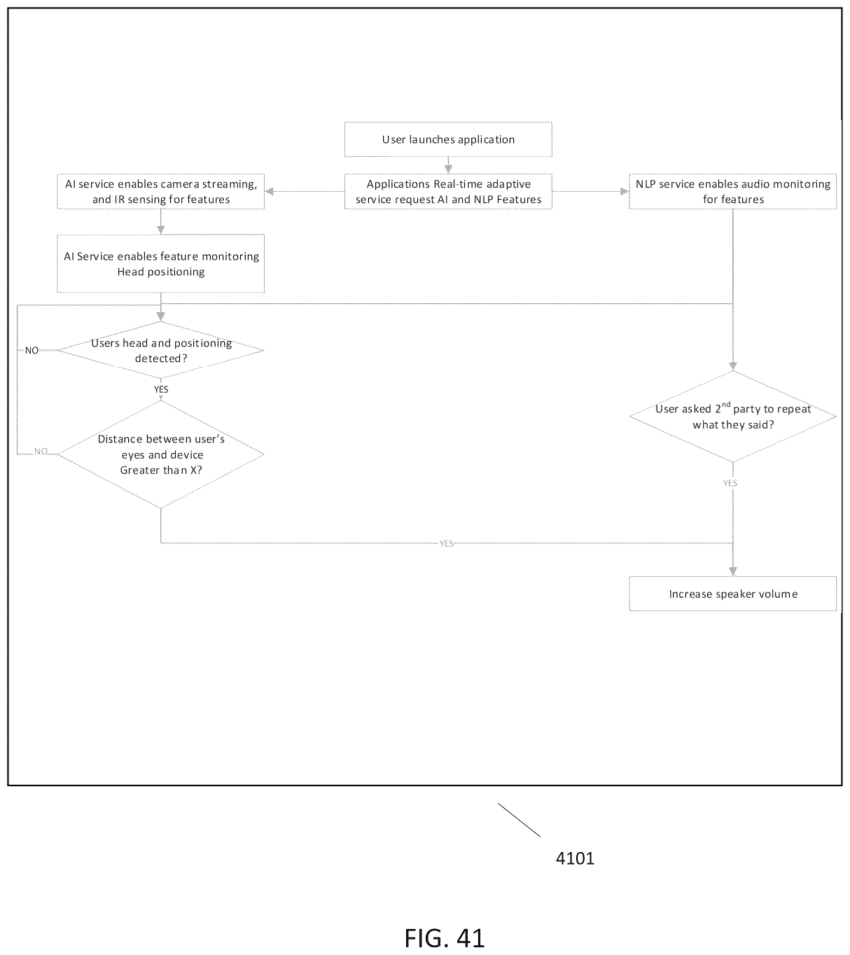

FIGS. 37 and 38 depict user interfaces 3701 and 3801 with regular sized GUI component 3702 and GUI component 3802, respectively, where GUI component 3802 is automatically enlarged based on monitoring of the patient interaction with the IMD-related application according to some embodiments. FIG. 39 depicts an example patient mobile device 3901 adapted according to some representative embodiments. Device 3901 includes an IMD-related application which responds to events from a real-time adaptive service configured to monitor user interaction with the IMD-related application. The service may process user interaction data using AI features related to squinting, head position/movement, gaze, and/or the like. Also, the service may process user interaction data using NLP features such as word-to-text processing of audio data. FIGS. 40 and 41 depict flowcharts 4001 and 4101 for processing user interaction data according to some representative embodiments.

In the above description of various embodiments of the present disclosure, it is to be understood that the terminology used herein is for the purpose of describing particular embodiments only and is not intended to be limiting of the invention. Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of this specification and the relevant art and may not be interpreted in an idealized or overly formal sense expressly so defined herein.

At least some example embodiments are described herein with reference to one or more circuit diagrams/schematics, block diagrams and/or flowchart illustrations. It is understood that such diagrams and/or flowchart illustrations, and combinations of blocks in the block diagrams and/or flowchart illustrations, can be implemented by any appropriate circuitry configured to achieve the desired functionalities. Accordingly, example embodiments of the present disclosure may be embodied in hardware and/or in software (including firmware, resident software, micro-code, etc.) operating in conjunction with suitable processing units or microcontrollers, which may collectively be referred to as “circuitry,” “a module” or variants thereof. An example processing unit or a module may include, by way of illustration, a general purpose processor, a special purpose processor, a conventional processor, a digital signal processor (DSP), a plurality of microprocessors, one or more microprocessors in association with a DSP core, a controller, a microcontroller, Application Specific Integrated Circuits (ASICs), Field Programmable Gate Array (FPGA) circuits, any other type of integrated circuit (IC), and/or a state machine, as well as programmable system devices (PSDs) employing system-on-chip (SoC) architectures that combine memory functions with programmable logic on a chip that is designed to work with a standard microcontroller. Example memory modules or storage circuitry may include volatile and/or non-volatile memories such as, e.g., random access memory (RAM), electrically erasable/programmable read-only memories (EEPROMs) or UV-EPROMS, one-time programmable (OTP) memories, Flash memories, static RAM (SRAM), etc.

Further, in at least some additional or alternative implementations, the functions/acts described in the blocks may occur out of the order shown in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated. Furthermore, although some of the diagrams include arrows on communication paths to show a primary direction of communication, it is to be understood that communication may occur in the opposite direction relative to the depicted arrows. Finally, other blocks may be added/inserted between the blocks that are illustrated.

It should therefore be clearly understood that the order or sequence of the acts, steps, functions, components or blocks illustrated in any of the flowcharts depicted in the drawing Figures of the present disclosure may be modified, altered, replaced, customized or otherwise rearranged within a particular flowchart, including deletion or omission of a particular act, step, function, component or block. Moreover, the acts, steps, functions, components or blocks illustrated in a particular flowchart may be inter-mixed or otherwise inter-arranged or rearranged with the acts, steps, functions, components or blocks illustrated in another flowchart in order to effectuate additional variations, modifications and configurations with respect to one or more processes for purposes of practicing the teachings of the present patent disclosure.

Although various embodiments have been shown and described in detail, the claims are not limited to any particular embodiment or example. None of the above Detailed Description should be read as implying that any particular component, element, step, act, or function is essential such that it must be included in the scope of the claims. Where example embodiments, arrangements or implementations describe a host of features, it should be understood that any one or more of the described features are optional depending on the context and/or unless expressed stated otherwise. Where the phrases such as “at least one of A and B” or phrases of similar import (e.g., “A and/or B”) are recited or described, such a phrase should be understood to mean “only A, only B, or both A and B.” Reference to an element in the singular is not intended to mean “one and only one” unless explicitly so stated, but rather “one or more.” Moreover, the terms “first,” “second,” and “third,” etc. employed in reference to elements or features are used merely as labels, and are not intended to impose numerical requirements, sequential ordering or relative degree of significance or importance on their objects. All structural and functional equivalents to the elements of the above-described embodiments that are known to those of ordinary skill in the art are expressly incorporated herein by reference and are intended to be encompassed by the present claims. Accordingly, those skilled in the art will recognize that the exemplary embodiments described herein can be practiced with various modifications and alterations within the spirit and scope of the claims appended below.

1. A method of remotely programming a medical device that provides therapy to a patient, comprising:
establishing a first communication between a patient controller (PC) device and the medical device, wherein the medical device provides therapy to the patient according to one or more programmable parameters, the PC device communicates signals to the medical device to set or modify the one or more programmable parameters, and the PC device comprises a video camera;
establishing a video connection between the PC device and a clinician programmer (CP) device of a clinician for a remote programming session in a second communication that includes an audio/video (A/V) session; and
modifying a value for one or more programmable parameters of the medical device according to signals from the CP device during the remote programming session;
wherein the method further comprises:
monitoring touch input on the PC device during the remote programming session;
detecting irregular movement in response to the monitoring; and
modifying, in response to the detecting, at least one graphical user interface (GUI) component on a patient application on the PC device to assist input from the patient.

2. The method of claim 1 wherein the modifying comprises enlarging the at least one GUI component.

3. The method of claim 1 wherein the modifying comprises enlarging text size.

4. The method of claim 1 wherein the detecting the irregular movement comprises detecting tremor in the patient.

5. A method of operating an application for a patient with an implantable medical device, comprising:
establishing a communication session with the implantable medical device with the application using wireless communication circuitry of a patient controller (PC) device;
receiving touch input by the application during the communication session to control application operations during the communication session;
monitoring touch input on the PC device during the communication session;
detecting irregular movement in response to the monitoring; and
modifying, in response to the detecting, at least one graphical user interface (GUI) component on a patient application on the PC device to assist input from the patient during the communication session.

6. The method of claim 5 further comprising:
receiving physiological data from the implantable medical device by the application during the communication session.

7. The method of claim 5 further comprising:
modifying one or more parameters of the implantable medical device for patient therapy.

8. The method of claim 5 wherein the modifying comprises enlarging the at least one GUI component.

9. The method of claim 5 wherein the modifying comprises enlarging text size.

10. The method of claim 5 wherein the detecting the irregular movement comprises detecting tremor in the patient.
